(19) United States
(12) Patent Application Publication
(10) Pub. No.: US 2024/0190889 A1
(43) Pub. Date: Jun. 13, 2024

(54) NEW CYCLOPENTA-THIENO-DIAZEPINE DERIVATIVES AS GABA A GAMMA1 POSITIVE ALLOSTERIC MODULATORS

(71) Applicant: Hoffmann-La Roche Inc., Little Falls, NJ (US)

(72) Inventors: Giuseppe CECERE, Basel (CH); Luca GOBBI, Buus (CH); Maria-Clemencia HERNANDEZ, Delémont (CH); Andreas KOBLET, Bottmingen (CH); Andres Miguel OLIVARES MORALES, Binningen (CH); Valerie RUNTZ-SCHMITT, Rixheim (FR); Nicolas ZORN, Durmenach (FR)

(73) Assignee: Hoffmann-La Roche Inc., Little Falls, NJ (US)

(21) Appl. No.: 18/429,832

(22) Filed: Feb. 1, 2024

Related U.S. Application Data
(63) Continuation of application No. PCT/EP2022/071608, filed on Aug. 2, 2022.

(30) Foreign Application Priority Data
Aug. 2, 2021 (EP) 21188998.5

(51) Int. Cl. C07D 495/14; A61K 31/551

(52) U.S. Cl. CPC C07D 495/14; A61K 31/551

(57) ABSTRACT

The invention provides heterocyclic compounds having the general formula (1), and pharmaceutically acceptable salts thereof, wherein the variables are as described herein.

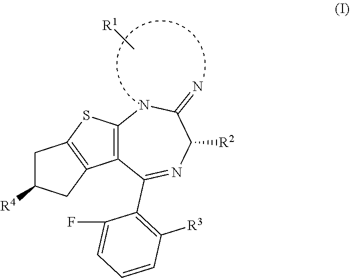

Further provided are pharmaceutical compositions including the compounds, processes of manufacturing the compounds and methods of using the compounds as medicaments, in particular methods of using the compounds for the treatment or prevention of acute neurological disorders, chronic neurological disorders and/or cognitive disorders. release wednesday

### CROSS REFERENCE TO RELATED APPLICATIONS

This application is a continuation of International Patent Application No. PCT/EP2022/071608, filed Aug. 2, 2022, which claims priority to European Patent Application Number 21188998.5 filed Aug. 2, 2021, both of which are incorporated herein by reference in their entirety.

### FIELD OF THE INVENTION

This invention relates to compounds useful for therapy or prophylaxis in a mammal, and in particular to new cyclopenta-thieno-diazepine derivatives that exhibit activity as GABA_{A} γ1 receptor positive allosteric modulators (PAMs) and are thus useful for the treatment or prophylaxis of GABA_{A} γ1 receptor related diseases or conditions.

### BACKGROUND OF THE INVENTION

Receptors for the major inhibitory neurotransmitter, gamma-aminobutyric acid (GABA), are divided into two main classes: (1) GABA_{A} receptors, which are members of the ligand-gated ion channel superfamily and (2) GABA_{B} receptors, which are members of the G-protein linked receptor family. The GABA_{A} receptor complex which is a membrane-bound heteropentameric protein polymer is composed principally of α, β and γ subunits. GABA_{A} receptors are ligand-gated chloride channels and the principal mediators of inhibitory neurotransmission in the human brain.

There are 19 genes encoding for GABA_{A} receptor subunits that assemble as pentamers with the most common stoichiometry being two α, two β and one γ subunit. GABA_{A} subunit combinations give rise to functional, circuit, and behavioral specificity. GABA_{A} receptors containing the γ1 subunit (GABA_{A} γ1) are of particular interest due to their enriched expression in the limbic system and unique physiological and pharmacological properties. The GABA_{A} γ1 subunit-containing receptors, while less abundant (around 5-10% of total expression of GABA_{A} receptors in the brain) than γ2 subunit-containing receptors exhibit an enriched brain mRNA and protein distribution in key brain areas such as extended amygdala (central, medial, and bed nucleus of the stria terminalis), lateral septum, hypothalamus, and pallidum/nigra. These structures form the interconnected core of a subcortical limbic circuit regulating motivated social and affective behaviors. In abnormal or disease conditions, hyper-recruitment of this circuit promotes anxiety, arousal, aggression, fear and defense while inhibiting foraging and social interactions.

Hyperactivity in limbic cortical regions (known to form a coordinated functional network with extended amygdala/hypothalamus regions) which are key areas for processing of social and emotionally relevant stimuli, is the common hallmark of a variety of psychiatric, neurological, neurodevelopmental, neurodegenerative, mood, motivational and metabolic disorders. In such a disease state, and given the characteristic anatomical distribution of the γ1 subunit-containing GABA_{A} receptors, a GABA_{A} γ1 positive allosteric modulator (PAM) may be an effective treatment as a symptomatic or disease-modifying agent.

Multiple lines of evidence suggest that an imbalance between excitatory/inhibitory (E/I) neurotransmission arising from dysfunction of GABAergic signaling system, the main inhibitory neurotransmitter system in the brain, to be at the core of the pathogenesis a variety of CNS disorders. Given the distribution and function of GABA_{A} γ1 subunit-containing receptors in the CNS, they are very attractive targets for restoring levels of inhibition within key brain circuits and consequently the E/I balance in these conditions.

A CNS disorders of particular interest in the context of the present invention is autism spectrum disorder (ASD), including its core symptoms and associated comorbidities, such as anxiety and irritability, social anxiety disorder (social phobia) and generalized anxiety disorder. ASD is a complex, heterogeneous neurodevelopmental disorder characterized by impairments in two core domains: impairments in social interaction and communication, and presence of repetitive or restricted behaviors, interests, or activities (American Psychiatric Association 2013).

No approved pharmacological treatment exists for core symptoms of social deficits and restricted/repetitive behaviour of ASD, while only inadequate therapeutic options are available for most of ASD's affective and physiological co-morbidities. As a result, this disorder continues to be an area of high unmet medical need. Current approved treatments for associated symptoms of ASD are limited to the antipsychotics (Risperidone and Aripiprazole) indicated for the treatment of irritability associated with ASD symptoms. Emerging evidence suggests that the GABAergic system, the main inhibitory neurotransmitter system in the brain, plays a key role in the pathophysiology of ASD.

Both genetic and imaging studies using positron emission tomography study (PET) and magnetic resonance spectroscopy (MRS) suggest alterations in GABAergic signaling in ASD. The gene encoding GABA_{A} γ1, GABRGI, is located on chromosome 4 (mouse Chr.5) in a cluster with genes encoding α2, α4 and β1 GABA_{A} receptor subunits. Rare CNVs, including inversion of chromosome 4p12 disrupting GABRGI have been observed in autistic siblings (Horike et al., 2006), as well as GABRGI loss in one case of ADHD. Mutations in 4p12 gene cluster have been linked to increased risk of anxiety, substance abuse and eating disorders—providing a link between GABRG¼p12 and affective dysfunction. MRS studies found altered GABA levels in ASD and in particular some recent studies showed reduced GABA and altered somatosensory function in children with ASD and. In line with these observations, a reduced number of inhibitory interneurons were found from postmortem tissues of ASD and TS patients. Furthermore, reduced GABA synthesizing enzymes, glutamic acid decarboxylase (GAD) 65 and 67 were found in parietal and cerebellar cortices of patients with autism. Strong evidence in humans points to specific dysfunction in ASD of the limbic cortical regions known to form a coordinated functional network with GABA_{A} γ1 subunit-containing extended amygdala/hypothalamus regions. These areas: Cortical/lateral amygdala. Insula, PFC, and Cingulate are recognized key for processing of social and emotionally relevant stimuli. While subcortical subnuclei that form specific partnerships with these areas, coordinating behavioural outcomes, are often difficult to study due to spatial resolution limitations, many lines of evidence point to hyper-recruitment of these cortical- to sub cortical connections in ASD. Moreover, recent high resolution studies provide a clear link between extended amygdala activity/functional connectivity and emotional state. Targeting such highly specified limbic subcortical regions, which exhibit substantial molecular and cellular diversity compared to the neocortex, will create a precision entry point for safe and specific therapeutic modulation of ASD-affected socio-affective circuits, while avoiding broad modulation of global brain state. Enhancement of GABA_{A} receptor activity by non-selective BZDs have been shown to ameliorate behavioral deficits in mouse models of ASD, however very narrow therapeutic margins were observed due to sedation mediated by the GABA_{A} aly2 subtype. These findings support the notion that rebalancing of GABAergic transmission via GABA_{A} γ1 receptors can improve symptoms in ASD without the side effects of non-selective benzodiazepines.

### SUMMARY OF THE INVENTION

Compounds of the invention are selective GABA_{A} γ1 receptor positive allosteric modulators (PAMs) that selectively enhance the function of γ1-containing GABA_{A} receptors by increasing GABAergic currents (influx of chloride) at a given concentration (e.g. EC₂₀) of gamma amino butyric acid (GABA). The compounds of the invention have high PAM efficacy and binding selectivity for the γ1-containing subtypes (α5γ1, α2γ1, α1γ1) relative to the y2-containing subtypes (e.g. α1γ2, α2γ2, α3γ2 and α5γ2). As such, compounds of the present invention are strongly differentiated from classical benzodiazepine drugs such as Alprazolam. Triazolam. Estazolam, and Midazolam, which are selective for the y2-containing GABA_{A} subtypes and possess low affinity for the γ1-containing subtypes. Compatible with the γ1-subtypes brain distribution, selective GABA_{A} Y1 PAMs will restore GABAergic signaling in key brain regions (e.g. extended amygdala: central, medial, and bed nucleus of the stria terminalis, lateral septum, hypothalamus, and pallidum/nigra) without the side-effects of non-selective GABA_{A} modulators (e.g. benzodiazepines).

The selective GABA_{A} Y1 PAMs described herein and their pharmaceutically acceptable salts and esters are useful, alone or in combination with other drugs, as disease-modifying or as symptomatic agents for the treatment or prevention of acute neurological disorders, chronic neurological disorders and/or cognitive disorders, including autism spectrum disorders (ASD),

Angelman syndrome, age-related cognitive decline, Rett syndrome, Prader-Willi syndrome, amyotrophic lateral sclerosis (ALS), fragile-X disorder, negative and/or cognitive symptoms associated with schizophrenia, tardive dyskinesia, anxiety, social anxiety disorder (social phobia), panic disorder, agoraphobia, generalized anxiety disorder, disruptive, impulse-control and conduct disorders, Tourette's syndrome (TS), obsessive-compulsive disorder (OCD), acute stress disorder, post-traumatic stress disorder (PTSD), attention deficit hyperactivity disorder (ADHD), sleep disorders, Parkinson's disease (PD), Huntington's chorea, Alzheimer's disease (AD), mild cognitive impairment (MCI), dementia, behavioral and psychological symptoms (BPS) in neurodegenerative conditions, multi-infarct dementia, agitation, psychosis, substance-induced psychotic disorder, aggression, eating disorders, depression, chronic apathy, anhedonia, chronic fatigue, seasonal affective disorder, postpartum depression, drowsiness, sexual dysfunction, bipolar disorders, epilepsy and pain.

In a first aspect, the present invention provides a compound of formula (I)

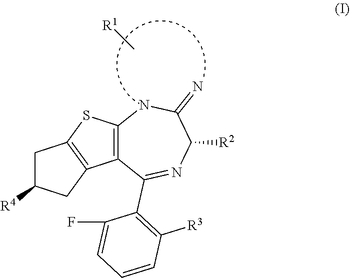

or a pharmaceutically acceptable salt thereof, wherein the variables are as defined herein.

In one aspect, the present invention provides a process of manufacturing the compounds of formula (I) described herein, wherein said process is as described in any one of Schemes 1 to 8 herein.

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides a compound of formula (I) as described herein, or a pharmaceutically acceptable salt thereof, for use in a method for treating or preventing acute neurological disorders, chronic neurological disorders and/or cognitive disorders in a subject.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 6 carbon atoms (“C₁-C₆-alkyl”), e.g., 1, 2, 3, 4, 5, or 6 carbon atoms. In some embodiments, the alkyl group contains 1 to 3 carbon atoms, e.g., 1, 2 or 3 carbon atoms. Some non-limiting examples of alkyl include methyl, ethyl, propyl, 2-propyl (isopropyl), n-butyl, iso-butyl, sec-butyl, tert-butyl, and 2,2-dimethylpropyl. Particularly preferred, yet non-limiting examples of alkyl include methyl and ethyl.

The term “alkoxy” refers to an alkyl group, as previously defined, attached to the parent molecular moiety via an oxygen atom. Unless otherwise specified, the alkoxy group contains 1 to 6 carbon atoms (“C₁-C₆-alkoxy”). In some preferred embodiments, the alkoxy group contains contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. A particularly preferred, yet non-limiting example of alkoxy is methoxy.

The term “halogen” or “halo” refers to fluoro (F), chloro (Cl), bromo (Br), or iodo (I). Preferably, the term “halogen” or “halo” refers to fluoro (F), chloro (Cl) or bromo (Br). Particularly preferred. yet non-limiting examples of “halogen” or “halo” are fluoro (F) and chloro (CI).

The term “cycloalkyl” as used herein refers to a saturated monocyclic or bicyclic hydrocarbon group of 3 to 10 ring carbon atoms (“C₃-C₁₀-cycloalkyl”). In some preferred embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. Preferably, the cycloalkyl group is a saturated monocyclic hydrocarbon group of 3 to 6 ring carbon atoms, e.g., of 3, 4, 5 or 6 carbon atoms. Some non-limiting examples of cycloalkyl include cyclopropyl, cyclobutyl, cyclopentyl, cyclohexyl, cycloheptyl, and spiro[2.3 ]hexan-5-γ1.

The term “aminoalkyl” refers to an alkyl group, wherein at least one of the hydrogen atoms of the alkyl group has been replaced by an amino group. Preferably, “aminoalkyl” refers to an alkyl group wherein 1, 2 or 3 hydrogen atoms of the alkyl group have been replaced by an amino group. Preferred, yet non-limiting examples of aminoalkyl are aminomethyl and 1-aminoethyl.

The term “heterocyclyl” or “heterocycloalkyl” refers to a saturated mono- or bicyclic, preferably monocyclic ring system of 3 to 14 ring atoms, preferably 3 to 10 ring atoms, more preferably 3 to 8 ring atoms wherein 1, 2, or 3 of said ring atoms are heteroatoms selected from N, O and S, the remaining ring atoms being carbon. Preferably, 1 to 2 of said ring atoms are selected from N and O, the remaining ring atoms being carbon. “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Some non-limiting examples of heterocyclyl groups include azetidin-3-yl: azetidin-2-yl: oxetan-3-yl: oxetan-2-yl: piperidyl: piperazinyl: pyrrolidinyl: 2-oxopyrrolidin-1-yl: 2-oxopyrrolidin-3-yl: 5-oxopyrrolidin-2-yl: 5-oxopyrrolidin-3-yl: 2-oxo-1-piperidyl: 2-oxo-3-piperidyl: 2-oxo-4-piperidyl: 6-oxo-2-piperidyl: 6-oxo-3-piperidyl: 1-piperidinyl: 2-piperidinyl: 3-piperidinyl: 4-piperidinyl: morpholino (e.g., morpholin-2-γ1 or morpholin-3-yl): thiomorpholino, pyrrolidinyl (e.g., pyrrolidin-3-yl): 3-azabicyclo[3.1.0]hexan-6-yl: 2,5-diazabicyclo[2.2.1 ]heptan-2-yl: 2-azaspiro[3.3]heptan-2-yl: 2,6-diazaspiro[3.3]heptan-2-yl: and 2,3,3a,4,6,6a-hexahydro-1H-pyrrolo[3,4-c]pyrrol-5-yl. A preferred, yet non-limiting example of heterocyclyl is thiomorpholino.

The term “heteroaryl” refers to a mono- or multivalent, monocyclic or bicyclic, preferably bicyclic ring system having a total of 5 to 14 ring members, preferably, 5 to 12 ring members, and more preferably 5 to 10 ring members, wherein at least one ring in the system is aromatic, and at least one ring in the system contains one or more heteroatoms. Preferably, “heteroaryl” refers to a 5-10 membered heteroaryl comprising 1, 2, 3 or 4 heteroatoms independently selected from O, S and N. Most preferably, “heteroaryl” refers to a 5-10 membered heteroaryl comprising 1 to 2 heteroatoms independently selected from O and N. Some non-limiting examples of heteroaryl include 2-pyridyl, 3-pyridyl, 4-pyridyl, pyrimidin-2-yl, pyrimidin-4-yl, pyrimidin-5-yl, pyrimidin-6-yl, indol-1-yl, 1H-indol-2-yl, 1H-indol-3-yl, 1H-indol-4-yl, 1H-indol-5-yl, 1H-indol-6-yl, 1H-indol-7-yl, 1,2-benzoxazol-3-yl, 1,2-benzoxazol-4-yl, 1,2-benzoxazol-5-yl, 1,2-benzoxazol-6-yl, 1,2-benzoxazol-7-yl, 1H-indazol-3-yl, 1H-indazol-4-yl, 1H-indazol-5-yl, 1H-indazol-6-yl, 1H-indazol-7-yl, pyrazol-1-yl, 1H-pyrazol-3-yl, 1H-pyrazol-4-yl, 1H-pyrazol-5-yl, pyrazin-3-yl, pyrazin-4-yl, imidazol-1-yl, 1H-imidazol-2-yl, 1H-imidazol-4-yl, 1H-imidazol-5-yl, oxazol-2-yl, oxazol-4-yl, oxazol-5-yl, thiazol-4-yl, 1,2,4-oxadiazol-3-yl, 1H-triazol-5-yl, 1H-triazol-4-yl, and triazol-1-yl.

Most preferably, “heteroaryl” refers to pyrazinyl.

The term “hydroxy” refers to an —OH group.

The term “oxo” refers to an oxygen atom that is bound to the parent moiety via a double bond (═O).

The term “amino” refers to an —NH₂ group.

The term “cyano” refers to a —CN (nitrile) group.

The term “haloalkyl” refers to an alkyl group, wherein at least one of the hydrogen atoms of the alkyl group has been replaced by a halogen atom, preferably fluoro. Preferably, “haloalkyl” refers to an alkyl group wherein 1, 2 or 3 hydrogen atoms of the alkyl group have been replaced by a halogen atom, most preferably fluoro. Non-limiting examples of haloalkyl are fluoromethyl, difluoromethyl, trifluoromethyl, trifluoroethyl, 2-fluoroethyl, and 2,2-difluoroethyl. A particularly preferred, yet non-limiting example of haloalkyl is trifluoromethyl.

The term “alkoxyalkyl” refers to an alkyl group, wherein at least one of the hydrogen atoms of the alkyl group has been replaced by an alkoxy group. Preferably, “alkoxyalkyl” refers to an alkyl group wherein 1, 2 or 3 hydrogen atoms of the alkyl group have been replaced by an alkoxy group. Most preferably, “alkoxyalkyl” refers to an alkyl group wherein 1 hydrogen atom of the alkyl group has been replaced by an alkoxy group. A preferred, yet non-limiting example of alkoxyalkyl is 2-methoxyethyl.

The term “haloalkoxy” refers to an alkoxy group, wherein at least one of the hydrogen atoms of the alkoxy group has been replaced by a halogen atom, preferably fluoro. Preferably, “haloalkoxy” refers to an alkoxy group wherein 1, 2 or 3 hydrogen atoms of the alkoxy group have been replaced by a halogen atom, most preferably fluoro. Particularly preferred, yet non-limiting examples of haloalkoxy are fluoromethoxy (FCH₂O—), difluoromethoxy (F₂CHO—), and trifluoromethoxy (F₃CO—).

The term “hydroxyalkyl” refers to an alkyl group, wherein at least one of the hydrogen atoms of the alkyl group has been replaced by a hydroxy group. Preferably, “hydroxyalkyl” refers to an alkyl group wherein 1, 2 or 3 hydrogen atoms, most preferably 1 hydrogen atom of the alkyl group have been replaced by a hydroxy group. Preferred, yet non-limiting examples of hydroxyalkyl are hydroxymethyl, hydroxyethyl (e.g. 2-hydroxyethyl), and 3-hydroxy-3-methyl-butyl.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid sulfuric acid, nitric acid, phosphoric acid and the like, in particular hydrochloric acid, and organic acids such as formic acid, acetic acid, trifluoroacetic acid, propionic acid, glycolic acid. pyruvic acid, oxalic acid, maleic acid, malonic acid, succinic acid, fumaric acid, tartaric acid, lactic acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochlorides, fumarates, formates, lactates (in particular derived from L-(+)-lactic acid), tartrates (in particular derived from L-(+)-tartaric acid) and trifluoroacetates.

The compounds of formula (I) can contain several asymmetric centers and can be present in the form of optically pure enantiomers, mixtures of enantiomers such as, for exampleracemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” or “prevention” as used herein includes: preventing or delaying the appearance of clinical symptoms of the state, disorder or condition developing in a subject and especially a human that may be afflicted with or predisposed to the state, disorder or condition but does not yet experience or display clinical or subclinical symptoms of the state, disorder or condition.

The term “subject” as used herein includes both humans and non-humans and includes but is not limited to humans, non-human primates, canines, felines, murines, bovines, equines, and porcines. In a particularly preferred embodiment, the term “subject” refers to humans.

The term “protecting group” (PG) denotes a group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups. Particular protecting groups are the tert-butoxycarbonyl (Boc), benzyloxycarbonyl (Cbz), fluoreny lmethoxycarbonyl (Fmoc) and benzyl (Bn) groups. Further particular protecting groups are the tert-butoxycarbonyl (Boc) and the fluoreny lmethoxycarbonyl (Fmoc) groups. More particular protecting group is the tert-butoxycarbonyl (Boc) group.

The abbreviation uM means microMolar and is equivalent to the symbol μM.

The abbreviation uL means microliter and is equivalent to the symbol μL.

The abbreviation ug means microgram and is equivalent to the symbol μg.

### Compounds of the Invention

In a first aspect, the present invention provides a compound of formula (I)

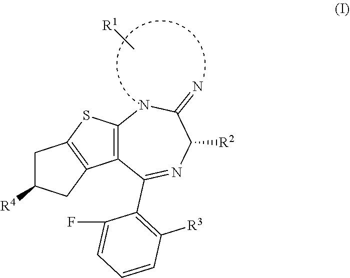

or a pharmaceutically acceptable salt thereof, wherein:

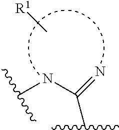

is selected from:

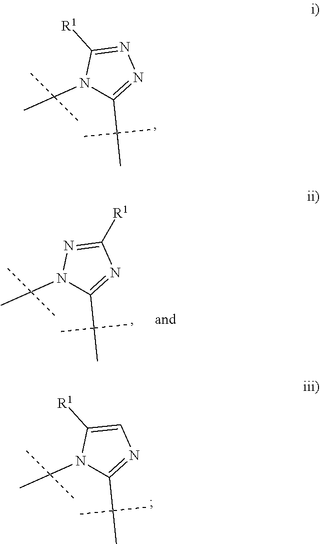

R¹ is selected from hydrogen, C₁-C₆-alkyl, C₁-C₆-alkoxy, C₁-C₆-alkoxy-C₁-C₆-alkyl, hydroxy-C₁-C₆-alkyl, C₃-C₁₀-cycloalkyl, amino-C₁-6-alkyl, 5-14-membered heteroaryl, 3-14-membered heterocycloalkyl, (3-14-membered heterocycloalkyl)—C(O)—, and —C(O)NR⁵R⁶; wherein said C₃-C₁₀-cycloalkyl, 5-14-membered heteroaryl and 3-14-membered heterocycloalkyl are optionally substituted by 1-3 substituents that are each independently selected from halogen, cyano, hydroxy, oxo, amino, C₁-C₆-alkyl, C₁-C₆-alkoxy, halo-C₁-C₆-alkyl, and halo-C₁-C₆-alkoxy;
R² is selected from hydrogen, C₁-C₆-alkyl, and C₁-C₆-alkoxy;
R³ is selected from chloro and fluoro;
R⁴ is selected from C₁-C₆-alkyl, halo-C₁-C₂-alkyl, and cyano; and
R⁵ and R⁶ are each independently selected from hydrogen, C₁-C₆-alkyl, and hydroxy-C₁-C₆-alkyl.

In a preferred embodiment, the present invention provides a compound of formula (I) as described herein, or a pharmaceutically acceptable salt thereof, wherein

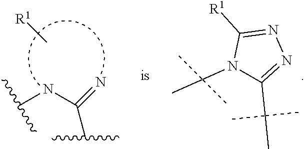

In one embodiment, the present invention provides a compound of formula (I) as described herein, or a pharmaceutically acceptable salt thereof, wherein:
R¹ is selected from C₁-C₆-alkyl, hydroxy-C₁-C₆-alkyl, C₃-C₁₀-cycloalkyl, 5-14-membered heteroaryl, 3-14-membered heterocycloalkyl, and —C(O)NR⁵R⁶; wherein said 3-14-membered heterocycloalkyl is substituted by 2 oxo substituents:
R⁵ is hydroxy -C₁-C₆-alkyl; and
R⁶ is hydrogen.

In one embodiment, the present invention provides a compound of formula (I) as described herein, or a pharmaceutically acceptable salt thereof, wherein:
R¹ is selected from C₁-C₆-alkyl, hydroxy-C₁-C₆-alkyl, C₃-C₁₀-cycloalkyl, 5-14-membered heteroaryl, (3-14-membered heterocycloalkyl)-C(O)—, and —C(O)NR⁵R⁶; wherein said 3-14-membered heterocycloalkyl is substituted by 2 oxo substituents;
R⁵ is hydroxy-C₁-C₆-alkyl; and
R⁶ is hydrogen.

In a preferred embodiment, the present invention provides a compound of formula (I) as described herein, or a pharmaceutically acceptable salt thereof, wherein R¹ is C₁-C₆-alkyl.

In a particularly preferred embodiment, the present invention provides a compound of formula (I) as described herein, or a pharmaceutically acceptable salt thereof, wherein R¹ is methyl.

In one embodiment, the present invention provides a compound of formula (I) as described herein, or a pharmaceutically acceptable salt thereof, wherein R² is selected from hydrogen and C₁-C₆-alkyl.

In a preferred embodiment, the present invention provides a compound of formula (I) as described herein, or a pharmaceutically acceptable salt thereof, wherein R² is C₁-C₆-alkyl.

In a particularly preferred embodiment, the present invention provides a compound of formula (I) as described herein, or a pharmaceutically acceptable salt thereof, wherein R² is methyl.

In a preferred embodiment, the present invention provides a compound of formula (I) as described herein, or a pharmaceutically acceptable salt thereof, wherein R⁴ is halo-C₁-C₆-alkyl.

In a particularly preferred embodiment, the present invention provides a compound of formula (I) as described herein, or a pharmaceutically acceptable salt thereof, wherein R⁴ is CHF₂.

In one embodiment, the present invention provides a compound of formula (I) as described herein, or a pharmaceutically acceptable salt thereof, wherein:
R¹ is selected from C₁-C₆-alkyl, hydroxy-C₁-C₆-alkyl, C₃-C₁₀-cycloalkyl, 5-14-membered heteroaryl, (3-14-membered heterocycloalkyl)-C(O)—, and —C(O)NR⁵R⁶; wherein said 3-14-membered heterocycloalkyl is substituted by 2 oxo substituents;
R² is selected from hydrogen and C₁-C₆-alkyl;
R⁵ is hydroxy-C₁-C₆-alkyl; and
R⁶ is hydrogen.

In a preferred embodiment, the present invention provides a compound of formula (I) as described herein, or a pharmaceutically acceptable salt thereof, wherein:

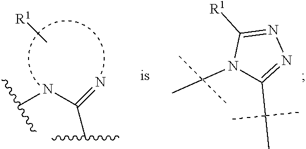

R¹ and R² are both C₁-C₆-alkyl; and
R⁴ is halo-C₁-C₆-alkyl.

In a particularly preferred embodiment, the present invention provides a compound of formula (I) as described herein, or a pharmaceutically acceptable salt thereof, wherein:

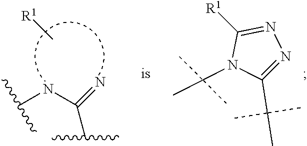

R¹ and R² are both methyl; and
R⁴ is CHF₂.

In one embodiment, the present invention provides a compound of formula (I) as described herein, or a pharmaceutically acceptable salt thereof, wherein said compound of formula (I) is selected from:

(13R)-9-(2-chloro-6-fluoro-phenyl)-3-methyl-16-thia-2,4,5,8-tetrazatetracyclo [8.6.0.02,6.011,15]hexadeca-1(10),3,5,8,11(15)-pentaene-13-carbonitrile:

(13R)-9-(2,6-difluorophenyl)-3-methyl-16-thia-2,4,5,8-tetrazatetracyclo [8.6.0.02,6.011,15]hexadeca-1(10),3,5,8,11(15)-pentaene-13-carbonitrile:

(13R)-13-(difluoromethyl)-9-(2,6-difluorophenyl)-3-methyl-16-thia-2,4,5,8-tetrazatetracyclo [8.6.0.02,6.011,15]hexadeca-1(10),3,5,8,11(15)-pentaene:

(13R)-9-(2-chloro-6-fluoro-phenyl)-13-(difluoromethyl)-3-methyl-16-thia-2,4,5,8-tetrazatetracyclo [8.6.0.02,6.011,15]hexadeca-1(10),3,5,8,11(15)-pentaene:

(7S,13R)-13-(difluoromethyl)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene:

(13R)-9-(2-chloro-6-fluoro-phenyl)-3-cyclopropyl-13-(difluoromethyl)-16-thia-2,4,5,8-tetrazatetracyclo [8.6.0.02,6.011,15]hexadeca-1(10),3,5,8,11(15)-pentaene:

(13R)-3-cyclopropyl-13-(difluoromethyl)-9-(2,6-difluorophenyl)-16-thia-2,4,5,8-tetrazatetracyclo [8.6.0.02,6.011,15]hexadeca-1(10),3,5,8,11(15)-pentaene:

(7S, 13R)-3-cyclopropyl-13-(difluoromethyl)-9-(2,6-difluorophenyl)-7-methyl-16-thia-2,4,5,8-tetrazatetracyclo [8.6.0.02,6.011,15]hexadeca-1(10),3,5,8,11(15)-pentaene:

(7S,13R)-9-(2-chloro-6-fluoro-phenyl)-13-(difluoromethyl)-3,7-dimethyl-16-thia-2,4,5,8-tetrazatetracyclo [8.6.0.02,6.011,15]hexadeca-1(10),3,5,8,11(15)-pentaene:

(13R)-13-(difluoromethyl)-9-(2,6-difluorophenyl)-3-pyridazin-3-yl-16-thia-2,4,5,8-tetrazatetracyclo [8.6.0.02,6.011,15]hexadeca-1(10),3,5,8,11(15)-pentaene:

(7S,13R)-9-(2-chloro-6-fluoro-phenyl)-3-cyclopropyl-13-(difluoromethyl)-7-methyl-16-thia-2,4,5,8-tetrazatetracyclo [8.6.0.02,6.011,15]hexadeca-1(10),3,5,8,11(15)-pentaene:

(7S,13R)-9-(2-chloro-6-fluoro-phenyl)-13-(difluoromethyl)-7-methyl-3-pyridazin-3-yl-16-thia-2,4,5,8-tetrazatetracyclo [8.6.0.02,6.011,15]hexadeca-1(10),3,5,8,11(15)-pentaene:

(7S,13R)-9-(2,6-difluorophenyl)-3,7-dimethyl-13-(trifluoromethyl)-16-thia-2,4,5,8-tetrazatetracyclo [8.6.0.02,6.011,15]hexadeca-1(10),3,5,8,11(15)-pentaene:

(4S,8R)-6-(2,6-difluorophenyl)-1,4-dimethyl-8-(trifluoromethyl)-8,9-dihydro-4H,7H-cyclopenta [4,5]thieno[3,2-f][1,2,4]triazolo[4,3-a][1,4]diazepine:

(13R)-9-(2,6-difluorophenyl)-3,13-dimethyl-16-thia-2,4,5,8-tetrazatetracyclo [8.6.0.02,6.011,15]hexadeca-1(10),3,5,8,11(15)-pentaene:

(7S,13R)-9-(2,6-difluorophenyl)-3,7,13-trimethyl-16-thia-2,4,5,8-tetrazatetracyclo [8.6.0.02,6.011,15]hexadeca-1(10),3,5,8,11(15)-pentaene:

(7S,13R)-13-(difluoromethyl)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene:

(7S,13S)-13-(difluoromethyl)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene:

(7S,13R)-13-(difluoromethyl)-9-(2,6-difluorophenyl)-N-(2-hydroxyethyl)-7-methyl-16-thia-2,3,5,8-tetrazatetracyclo [8.6.0.02,6.011,15]hexadeca-1(10),3,5,8,11(15)-pentaene-4-carboxamide:

[(7S,13R)-13-(difluoromethyl)-9-(2,6-difluorophenyl)-7-methyl-16-thia-2,3,5,8-tetrazatetracyclo [8.6.0.02,6.011, 15]hexadeca-1(10),3,5,8,11(15)-pentaen-4-yl]-(1,1-dioxo-1,4-thiazinan-4-yl)methanone:

(7S,13R)-13-(difluoromethyl)-9-(2,6-difluorophenyl)-3,7-dimethyl-16-thia-2,5,8-triazatetracyclo [8.6.0.02,6.011, 15]hexadeca-1(10),3,5,8,11(15)-pentaene:

[(7S,13R)-13-(difluoromethyl)-9-(2,6-difluorophenyl)-7-methyl-16-thia-2,5,8-triazatetracyclo [8.6.0.02,6.011, 15]hexadeca-1(10),3,5,8, 11(15)-pentaen-3-yl]methanol; and

[(7S,13S)-13-(difluoromethyl)-9-(2,6-difluorophenyl)-7-methyl-16-thia-2,5,8-triazatetracyclo [8.6.0.02,6.011, 15]hexadeca-1(10),3,5,8,11(15)-pentaen-3-yl]methanol.

In a preferred embodiment, the present invention provides a compound of formula (I) as described herein, or a pharmaceutically acceptable salt thereof, wherein said compound of formula (I) is selected from:

(7S,13R)-13-(difluoromethyl)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene; and

(7S,13R)-9-(2-chloro-6-fluoro-phenyl)-13-(difluoromethyl)-3,7-dimethyl-16-thia-2,4,5,8-tetrazatetracyclo [8.6.0.02,6.011,15]hexadeca-1(10),3,5,8,11(15)-pentaene.

In a particularly preferred embodiment, the present invention provides a compound of formula (I) as described herein, or a pharmaceutically acceptable salt thereof, wherein said compound of formula (I) is (7S,13R)-13-(difluoromethyl)-9-(2,6-difluorophenyl)-3,7-dimethyl-16-thia-2,4,5,8-tetrazatetracyclo [8.6.0.02,6.011, 15]hexadeca-1(10),3,5,8,11(15)-pentaene.

In a particularly preferred embodiment, the present invention provides a compound of formula (I) as described herein, or a pharmaceutically acceptable salt thereof, wherein said compound of formula (I) is (7S,13R)-9-(2-chloro-6-fluoro-phenyl)-13-(difluoromethyl)-3,7-dimethyl-16-thia-2,4,5,8-tetrazatetracyclo [8.6.0.02,6.011,15]hexadeca-1(10),3,5,8,11(15)-pentaene.

In one embodiment, the present invention provides pharmaceutically acceptable salts of the compounds of formula (I) as described herein, especially pharmaceutically acceptable salts selected from hydrochlorides, fumarates, lactates (in particular derived from L-(+)-lactic acid), tartrates (in particular derived from L-(+)-tartaric acid) and trifluoroacetates. In yet a further particular embodiment, the present invention provides compounds according to formula (I) as described herein (i.e., as “free bases” or “free acids”, respectively).

In some embodiments, the compounds of formula (I) are isotopically-labeled by having one or more atoms therein replaced by an atom having a different atomic mass or mass number. Such isotopically-labeled (i.e., radiolabeled) compounds of formula (I) are considered to be within the scope of this disclosure. Examples of isotopes that can be incorporated into the compounds of formula (I) include isotopes of hydrogen, carbon, nitrogen, oxygen, phosphorous, sulfur, fluorine, chlorine, and iodine, such as, but not limited to, ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Certain isotopically-labeled compounds of formula (I), for example, those incorporating a radioactive isotope, are useful in drug and/or substrate tissue distribution studies. The radioactive isotopes tritium, i.e. ³H, and carbon-14, i.e., ¹⁴C, are particularly useful for this purpose in view of their ease of incorporation and ready means of detection. For example, a compound of formula (I) can be enriched with 1, 2, 5, 10, 25, 50, 75, 90, 95, or 99 percent of a given isotope.

Substitution with heavier isotopes such as deuterium, i.e. H, may afford certain therapeutic advantages resulting from greater metabolic stability, for example, increased in vivo half-life or reduced dosage requirements.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of formula (I) can generally be prepared by conventional techniques known to those skilled in the art or by processes analogous to those described in the Examples as set out below using an appropriate isotopically-labeled reagent in place of the non-labeled reagent previously employed.

### Processes of Manufacturing

Processes for the manufacture of the compound of formula (I) as described herein are also an object of the invention.

The preparation of compounds of formula (I) of the present invention may be carried out in sequential or convergent synthetic routes. Syntheses of the compounds of the invention are shown in the following schemes. The skills required for carrying out the reactions and purifications of the resulting products are known to those skilled in the art. The substituents and indices used in the following description of the processes have the significance given herein before and in the claims, unless indicated to the contrary. In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 3rd Edition, Richard C. Larock. John Wiley & Sons, New York, NY. 2018). It is convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux temperature. The time required for the reaction may also vary widely, depending on many factors, notably the reaction temperature and the nature of the reagents. However, a period of from 0.5 h to several days will usually suffice to yield the described intermediates and compounds. The reaction sequence is not limited to the one displayed in the schemes, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The preparation of compounds of formula (I) of the present invention may be carried out in sequential or convergent synthetic routes. Syntheses of the invention are shown in the following general schemes. The skills required for carrying out the reactions and purifications of the resulting products are known to those skilled in the art. The substituents and indices used in the following description of the processes have the significance given herein before unless indicated to the contrary.

In more detail, the compounds of formula (I) can be manufactured by the methods given below, by the methods given in the examples or by analogous methods. Appropriate reaction conditions for the individual reaction steps are known to a person skilled in the art. The reaction sequence is not limited to the one displayed in schemes 1-8, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The present compounds of formula (Ia) and their pharmaceutically acceptable salts can be prepared by the process described in Scheme 1.

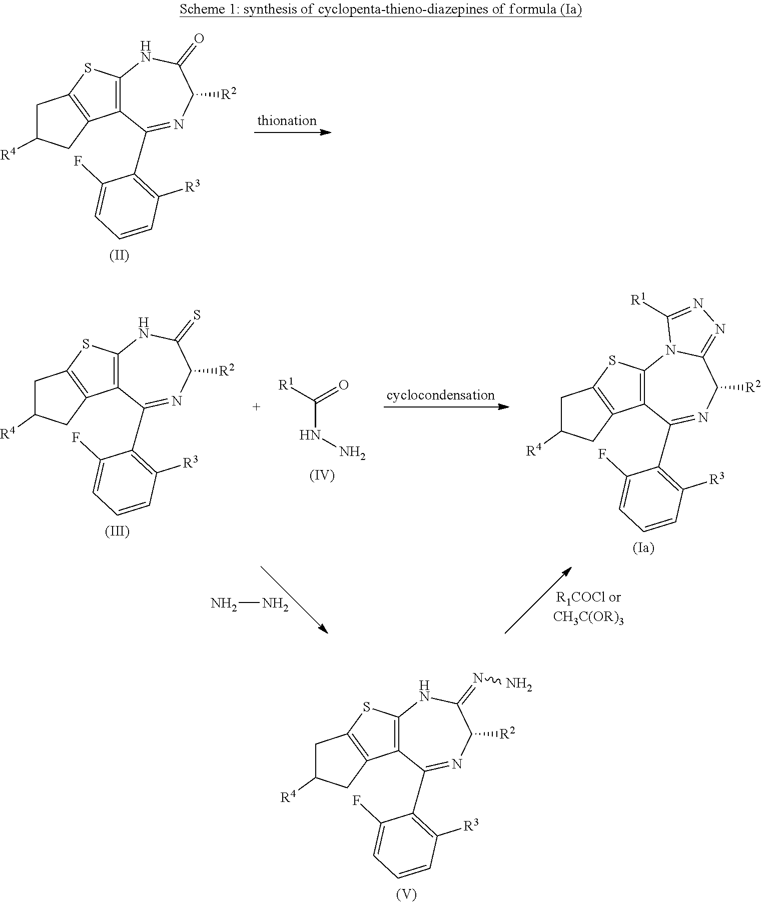

According to Scheme 1, a compound of formula (Ia) can be prepared in two steps starting from lactames of formula (II). Following thionation reaction using Lawesson's reagent or P2Ss, lactames (II) are converted to corresponding thiolactames (III). Their reaction with hydrazides (IV) via a Pellizzari type process yields 1,2,4-triazoles of general formula (Ia). In alternative, 1,2,4-triazoles (la) can be obtained by reaction between thiolactames (II) and hydrazine to form hydrazones (V) followed by treatment with trialkyl orthoacetate or acid chloride.

Synthesis of lactames (II) is highlighted in Scheme 2.

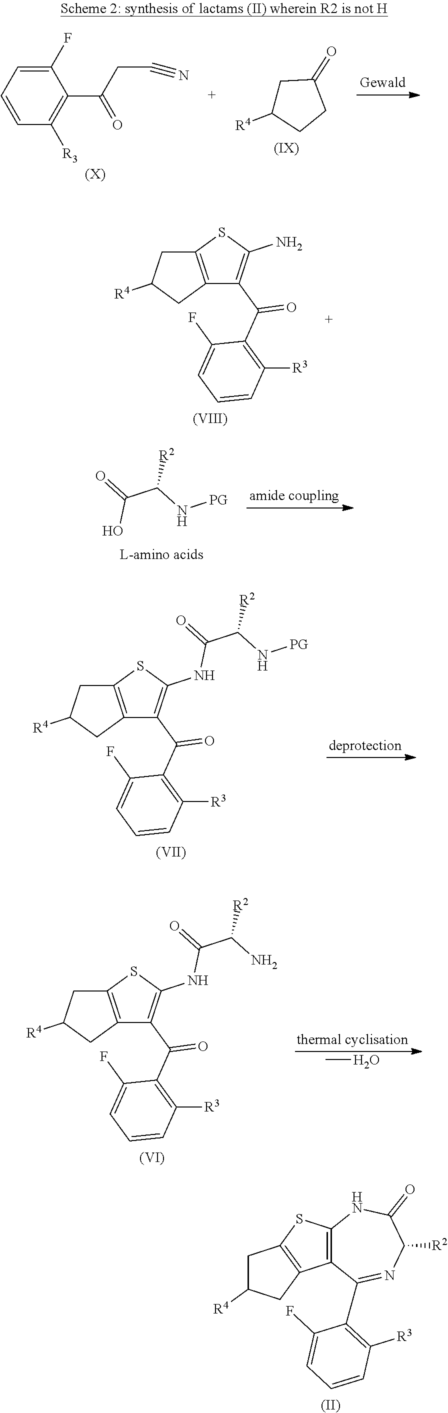

Commercially available nitriles (X) can be reacted with cyclopentanones (IX) under conventional Gewald reaction conditions to yield 2-amino-thiophenes of formula (VIII). Notably, in the Gewald reaction, a mixture of regioisomers is formed (from 20:1 to 1:1) depending on the substituent at R⁴.

Conveniently, the required regioisomer can be obtained pure after chromatographic removal of the undesired minor isomer and this can be performed at any stage of the synthesis.

Compounds of formula (VII) can be prepared by amide coupling reaction between 2-amino-thiophenes (VIII) and N-Boc or N-Cbz protected L-amino acids upon exposure to phosphoryl chloride (POC₁₃), or by other methods known to those skilled in the art. Removal of N-Boc or N-Cbz protecting group can be effected with mineral acids (e.g. HCl) or organic acids (e.g. trifluoroacetic acid) to yield amines of formula (VI). In case of N-Cbz protected intermediates (VII), the deprotection reaction can be better accomplished using iodotrimethylsilane.

Final intramolecular condensation reaction promoted by acidic media (e.g, silica or acetic acid) and heat (80-110° C.) provides desired lactam building block of formula (II).

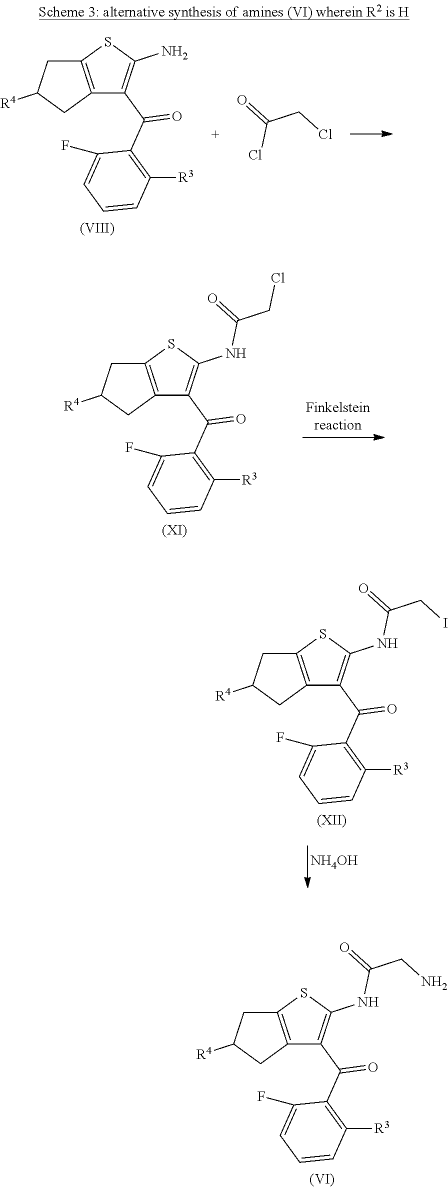

In alternative, compounds of formula (VI) wherein R² is H can be prepared according to a process described in Scheme 3. Conveniently, 2-amino-thiophenes (VIII) can be treated with chloroacetyl chloride to yield alkyl chlorides of formula (XI). These can be converted to corresponding alkyl iodides (XII) by a Finkelstein reaction. Final treatment with aqueous ammonia provides access to amines of formula (VI).

Final derivatives of formula (Ia) can be synthesized in three steps from esters of formula (XIII) (Scheme 4). In this case, esters (XIII) can be reduced with sodium borohydride to corresponding primary alcohols (XIV). Final derivatives (Ia) are obtained in two steps by Dess-Martin oxidation of alcohols (XIV) to aldehydes (XV), followed by fluorination reaction by exposure to (diethylamino)difluorosulfonium tetrafluoroborate.

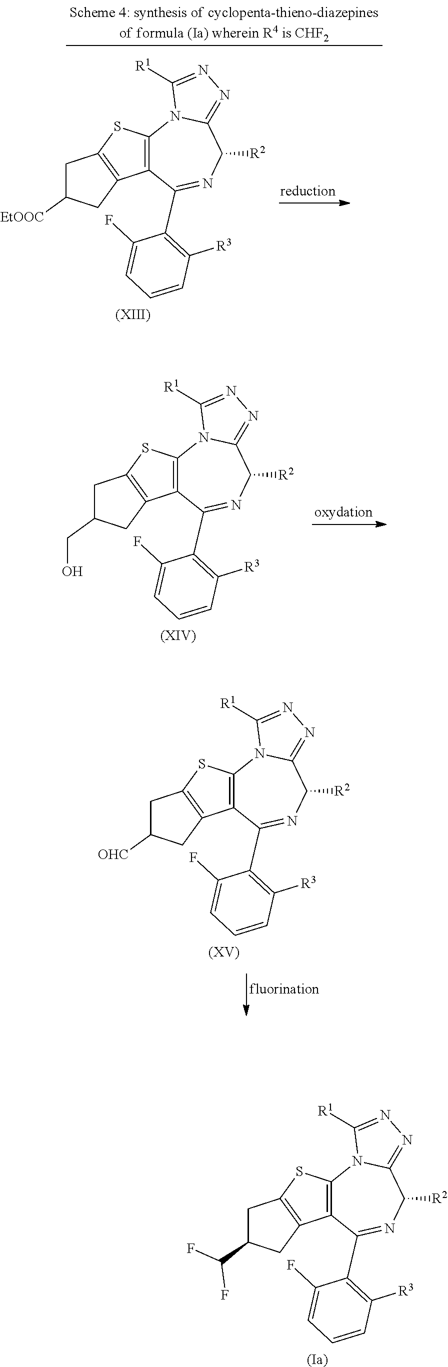

In certain embodiments of the invention, where R is methyl, compounds of formula (la) can be prepared in three steps from alcohols of formula (XIV) by the process described below (Scheme 5). Alcohols (XIV) can be converted into corresponding mesylates (XVI) by reaction with MsCl in presence of a base (e.g. Et₃N). Following an SN2 nucleophilic substitution reaction mesylates (XVI) are converted into iodoalkanes (XVII) which are reduced to final derivatives (Ia) by standard hydrogenation reaction.

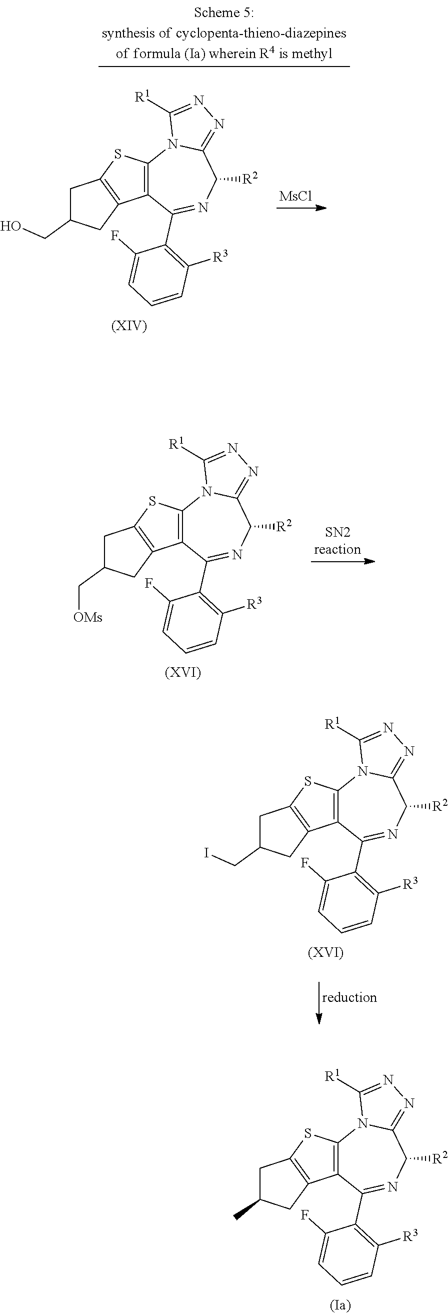

In certain embodiments of the invention, regioisomeric triazoles of formula (Ib) can be prepared from lactams (II) by the process described below (Scheme 6). Electrophilic amination of lactams (II) using and O-(diphenylphosphinyl)hydroxylamine yields intermediates of formula (XVIII).

Their final thermal cyclocondensation reaction with imidates (XIX) provides 1,2,4-triazoles (Ib). Final derivatives where R⁴ is CHF₂ are prepared using the same reaction conditions described in Scheme 4.

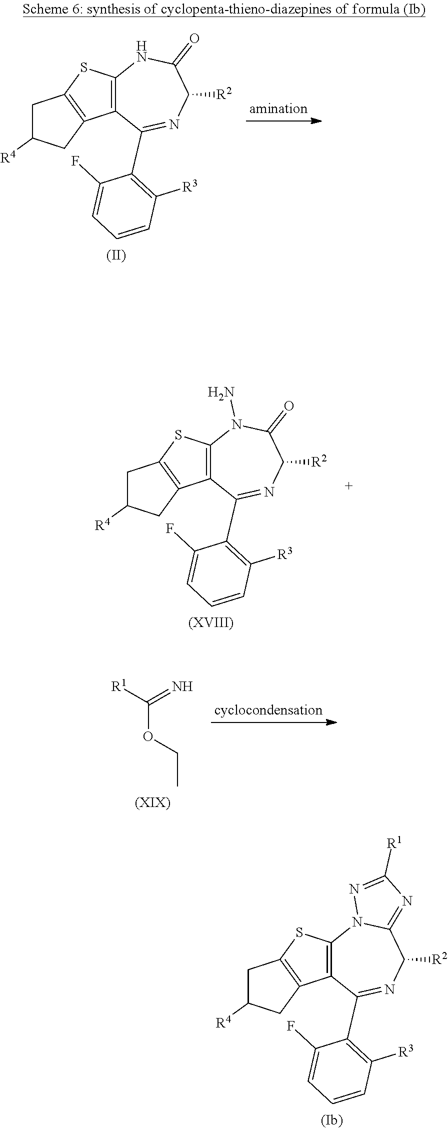

In certain embodiments of the invention where R¹ is an amide, compounds of formula (Ib) can be prepared according to Scheme 7. Ethyl esters (XX) are saponified to corresponding acids of formula (XXI) under basic conditions (NaOH). Final derivatives (Ib) are obtained by standard amide coupling reaction between acids (XXI) and amines HNR⁵R⁶ (XXII).

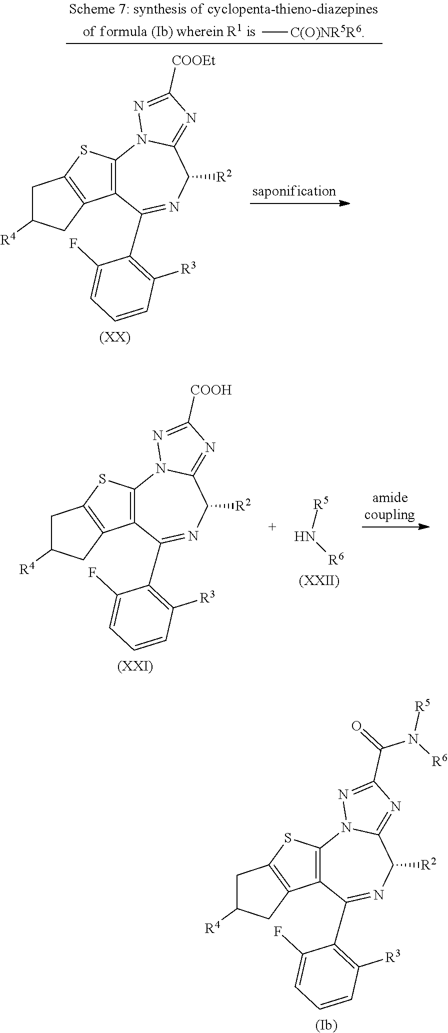

In further embodiments of the invention, imidazoles of formula (Ic) can be prepared as described below (Scheme 8). Thiolactams (III) are reacted with ammonia to form amidines of formula (XXIII). Following a reaction with propargylamine under acid catalysis, amidines (XXIII) can be converted to methyl imidazoles (Ic′). Their final oxidation to primary alcohols of formula (Ic) can be accomplished by reaction with selenium dioxide.

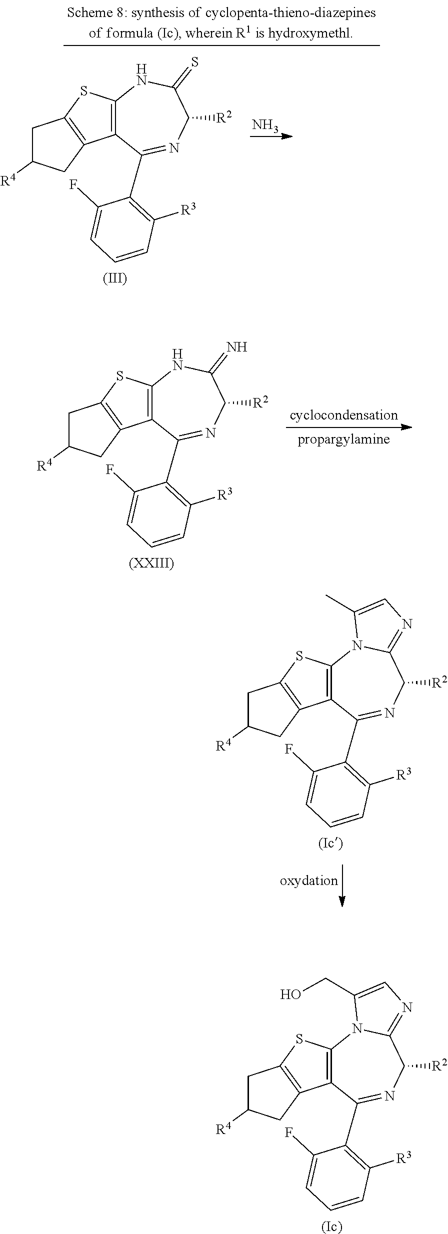

Notably, in the processes described in Scheme 1, 2, 6, 7 and 8, racemization at the chiral center occurs to various extents (20-100%), depending on specific reaction conditions adopted. As a result, chiral purification (e.g, by HPLC or SFC) of final derivatives of formula (I), is required to obtain single enantiomers (enantiomeric excess (ee) above 97%).

In one aspect, the present invention provides a process of manufacturing the compounds of formula (I) described herein, wherein said process is as described in any one of Schemes 1 to 8 above.

In a further aspect, the present invention provides a compound of formula (I) as described herein, or a pharmaceutically acceptable salt thereof, when manufactured according to the processes disclosed herein.

### Using the Compounds of the Invention

As explained in the background section and illustrated in the experimental section, the compounds of formula (I) and their pharmaceutically acceptable salts possess valuable pharmacological properties that make them useful for the treatment or prevention of diseases or condictions that are associated with the GABA_{A} γ1 receptor.

In one aspect, the present invention provides a compound of formula (I) as described herein, or a pharmaceutically acceptable salt thereof, for use as therapeutically active substance.

In a further aspect, the present invention provides a method for treating or preventing acute neurological disorders, chronic neurological disorders and/or cognitive disorders in a subject, said method comprising administering an effective amount of a compound of formula (I) as described herein, or a pharmaceutically acceptable salt thereof, or a pharmaceutical composition described herein, to the subject.

In a further aspect, the present invention provides the use of a compound of formula (I) as described herein, or a pharmaceutically acceptable salt thereof, or a pharmaceutical composition described herein, in a method for treating or preventing acute neurological disorders, chronic neurological disorders and/or cognitive disorders in a subject.

In a further aspect, the present invention provides a compound of formula (I) as described herein, or a pharmaceutically acceptable salt thereof, or a pharmaceutical composition described herein, for use in a method for treating or preventing acute neurological disorders, chronic neurological disorders and/or cognitive disorders in a subject.

In a further aspect, the present invention provides the use of a compound of formula (I) as described herein, or a pharmaceutically acceptable salt thereof, for the manufacture of a medicament for the treatment of prevention of acute neurological disorders, chronic neurological disorders and/or cognitive disorders.

In one embodiment, said acute neurological disorders, chronic neurological disorders and/or cognitive disorders are selected from autism spectrum disorders (ASD). Angelman syndrome, age-related cognitive decline. Rett syndrome, Prader-Willi syndrome, amyotrophic lateral sclerosis (ALS), fragile-X disorder, negative and/or cognitive symptoms associated with schizophrenia. tardive dyskinesia, anxiety, social anxiety disorder (social phobia), panic disorder, agoraphobia. generalized anxiety disorder, disruptive, impulse-control and conduct disorders. Tourette's syndrome (TS), obsessive-compulsive disorder (OCD), acute stress disorder, post-traumatic stress disorder (PTSD), attention deficit hyperactivity disorder (ADHD), sleep disorders, Parkinson's disease (PD), Huntington's chorea, Alzheimer's disease (AD), mild cognitive impairment (MCI), dementia, behavioral and psychological symptoms (BPS) in neurodegenerative conditions, multi-infarct dementia, agitation, psychosis, substance-induced psychotic disorder, aggression, eating disorders, depression, chronic apathy, anhedonia, chronic fatigue, seasonal affective disorder, postpartum depression, drowsiness, sexual dysfunction, bipolar disorders, epilepsy and pain.

In one embodiment, said acute neurological disorders, chronic neurological disorders and/or cognitive disorders are selected from Alzheimer's disease, mild cognitive impairment (MCI), age-related cognitive decline, negative and/or cognitive symptoms associated with schizophrenia. bipolar disorders, autism spectrum disorder (ASD), Angelman syndrome, Rett syndrome, Prader-Willi syndrome, epilepsy, post-traumatic stress disorder (PTSD), amyotrophic lateral sclerosis (ALS), and fragile-X disorder.

In a preferred embodiment, said acute neurological disorders, chronic neurological disorders and/or cognitive disorders are selected from autism spectrum disorder (ASD), Angelman syndrome, Alzheimer's disease, negative and/or cognitive symptoms associated with schizophrenia and post-traumatic stress disorder (PTSD).

In a preferred embodiment, said acute neurological disorders, chronic neurological disorders and/or cognitive disorders are selected from autism spectrum disorder (ASD), Rett syndrome, post-traumatic stress disorder and fragile-X disorder.

In a particularly preferred embodiment, said acute neurological disorders, chronic neurological disorders and/or cognitive disorders are autism spectrum disorder (ASD).

In a further particularly preferred embodiment, said acute neurological disorders, chronic neurological disorders and/or cognitive disorders are autism spectrum disorder (ASD), targeting core symptoms and associated comorbidities, such as anxiety and irritability, social anxiety disorder (social phobia) and generalized anxiety disorder.

### Pharmaceutical Compositions and Administration

In one aspect, the present invention provides pharmaceutical compositions comprising compounds of formula (I) or their pharmaceutically acceptable salts as defined herein and one or more pharmaceutically acceptable excipients. Exemplary pharmaceutical compositions are described in the Example section below.

In a further aspect, the present invention relates to pharmaceutical compositions comprising compounds of formula (I) or their pharmaceutically acceptable salts as defined above and one or more pharmaceutically acceptable excipients for the treatment or prevention of acute neurological disorders, chronic neurological disorders and/or cognitive disorders.

The compounds of formula (I) and their pharmaceutically acceptable salts can be used as medicaments (e.g, in the form of pharmaceutical preparations). The pharmaceutical preparations can be administered internally, such as orally (e.g, in the form of tablets, coated tablets, dragées, hard and soft gelatin capsules, solutions, emulsions or suspensions), nasally (e.g, in the form of nasal sprays) or rectally (e.g, in the form of suppositories). However, the administration can also be effected parentally, such as intramuscularly or intravenously (e.g, in the form of injection solutions or infusion solutions).

The compounds of formula (I) and their pharmaceutically acceptable salts can be processed with pharmaceutically inert, inorganic or organic excipients for the production of tablets, coated tablets, dragées and hard gelatin capsules. Lactose, corn starch or derivatives thereof, talc, stearic acid or its salts etc, can be used, for example, as such excipients for tablets, dragées and hard gelatin capsules.

Suitable excipients for soft gelatin capsules are, for example, vegetable oils, waxes, fats, semi-solid substances and liquid polyols, etc.

Suitable excipients for the production of solutions and syrups are, for example, water, polyols, saccharose, invert sugar, glucose, etc.

Suitable excipients for injection solutions are, for example, water, alcohols, polyols, glycerol, vegetable oils, etc.

Suitable excipients for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g, about 300 mg per person), divided into preferably 1-3 individual doses, which can consist, for example, of the same amounts, should be appropriate. It will, however, be clear that the upper limit given herein can be exceeded when this is shown to be indicated.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Example 1

### (13R)-9-(2-chloro-6-fluoro-phenyl)-3-methyl-16-thia-2,4,5,8-tetrazatetracyclo [8.6.0.02,6.011,15]hexadeca-1(10),3,5,8,11(15)-pentaene-13-carbonitrile

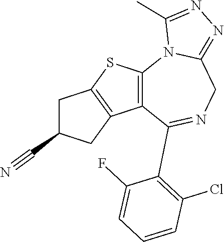 [SMILES: Cc1nnc2n1-c1sc3c(c1C(c1c(F)cccc1Cl)=NC2)C[C@@H](C#N)C3]

a) ethyl 2-amino-3-(2-chloro-6-fluoro-benzoyl)-5,6-dihydro-4H-cyclopenta[b]thiophene-5-carboxylate

To a solution of 3-(2-chloro-6-fluoro-phenyl)-3-oxo-propanenitrile (9.12 g, 46.2 mmol) in anhydrous ethanol (139 mL) was added ethyl 3-oxocyclopentanecarboxylate (7.21 g, 46.2 mmol), morpholine (4.02 ml, 46.2 mmol) and sulfur (1.48 g, 46.2 mmol). The reaction mixture was stirred at 60 ºC for 1.5 h, before being concentrated in vacuo. The crude residue was adsorbed on ISOLUTE® HM-N and purified by flash column chromatography (silica, 30 to 80% ethyl acetate in heptane) to afford the title compound (14.2 g, 83%) as a yellow oil, containing ca. 20% of ethyl 2-amino-3-(2-chloro-6-fluoro-benzoyl)-5,6-dihydro-4H-cyclopenta[b]thiophene-6-carboxylate. MS: 368.1 ([{³⁵Cl}M+H]⁺), 370.0 ([{³⁷Cl}M+H]⁺), ESI pos.

b) ethyl 2-[(2-chloroacetyl)amino]-3-(2-chloro-6-fluoro-benzoyl)-5,6-dihydro-4H-cyclopenta [b]thiophene-5-carboxylate

To a solution of ethyl 2-amino-3-(2-chloro-6-fluoro-benzoyl)-5,6-dihydro-4H-cyclopenta [b]thiophene-5-carboxylate (14.15 g, 38.5 mmol) in tetrahydrofuran (195 mL) was added K₂CO₃ (7.98 g, 57.7 mmol), followed by a dropwise addition of 2-chloroacetyl chloride (9.39 mL, 115 mmol) in tetrahydrofuran (43 mL). The reaction mixture was stirred at room temperature for 1 h, before being slowly poured into water (600 mL). Dichloromethane (250 mL) was added and the phases were separated. The aqueous layer was extracted with further dichloromethane (200 mL). The combined organic layers were dried (Na₂SO₄), filtered and concentrated in vacuo to afford the title compound (23.6 g, 100%) as an orange oil, containing ca.

30% of ethyl 2-[(2-chloroacetyl)amino]-3-(2-chloro-6-fluoro-benzoyl)-5,6-dihydro-4H-cyclopenta [b]thiophene-6-carboxylate. MS: 444.1 ([{³⁵Cl, ³⁵Cl}M+H]⁺), 446.1 ([{³⁵Cl, ³⁷Cl}M+H]⁺), ESI pos.

c) ethyl 2-[(2-aminoacetyl)amino]-3-(2-chloro-6-fluoro-benzoyl)-5,6-dihydro-4H-cyclopenta [b]thiophene-5-carboxylate

To a solution of ethyl 2-[(2-chloroacetyl)amino]-3-(2-chloro-6-fluoro-benzoyl)-5,6-dihydro-4H-cyclopenta [b]thiophene-5-carboxylate (23.6 g, 38.3 mmol) in acetone (210 mL) was added NaI (6.89 g, 46 mmol). The mixture was stirred at 60° C. for 22 h, then a further amount of Nal (2.3 g. 15.3 mmol) was added. The reaction mixture was stirred for 5 h, before being allowed to cool down to room temperature. The mixture was filtered directly through a sintered funnel and the filter cake was rinsed with acetone (2×40 mL). The filtrate was concentrated in vacuo to provide a red oil which was dissolved in dichloromethane (152 mL). The resulting solution was charged on a dropping funnel and slowly added into aqueous ammonium hydroxide (25 wt. %, 161 mL, 1.03 mol). The biphasic mixture was stirred slowly (without mixing of the phases) at room temperature for 5 days. The phases were separated. The aqueous layer was extracted with dichloromethane (2×100 mL). The combined organic layers were washed with brine, dried (Na₂SO₄), filtered and concentrated in vacuo. The crude material was adsorbed on ISOLUTE® HM-N and purified by flash column chromatography (silica, 20 to 70% ethyl acetate in heptane) to afford the title compound (6.95 g, 43%) as an orange oil, containing ca. 30% of ethyl 2-[(2-aminoacetyl)amino]-3-(2-chloro-6-fluoro-benzoyl)-5,6-dihydro-4H-cyclopenta [b]thiophene-6-carboxylate. MS: 425.0 ([{³⁵Cl}M+H]⁺), 427.0 ([{³Cl}M+H]⁺). ESI pos.

d) ethyl 13-(2-chloro-6-fluoro-phenyl)-10-oxo-7-thia-9,12-diazatricyclo[6.5.0.02,6]trideca-1(8), 2(6), 12-triene-4-carboxylate

To a suspension of ethyl 2-[(2-aminoacetyl)amino]-3-(2-chloro-6-fluoro-benzoyl)-5,6-dihydro-4H-cyclopenta [b]thiophene-5-carboxylate (4.74 g, 11.2 mmol) in toluene (279 mL) was added silica (13.4 g, 223 mmol). The mixture was stirred at 90° C. for 98 h, then a further amount of silica (5.36 g, 89.3 mmol) was added. The reaction mixture was stirred for 40 h, before being allowed to cool down to room temperature. The mixture was filtered directly through a sintered funnel and the filter cake (silica) was rinsed with ethyl acetate (2×100 mL). The filtrate was concentrated in vacuo and the crude material was adsorbed on ISOLUTE® HM-N and purified by flash column chromatography (silica, 0 to 100% ethyl acetate in heptane) to afford the title compound (2.57 g. 57%) as a brown oil, containing ca. 30% of ethyl 13-(2-chloro-6-fluoro-phenyl)-10-oxo-7-thia-9.12-diazatricyclo [6.5.0.02,6]trideca-1(8),2(6), 12-triene-5-carboxylate. MS: 407.2 ([{³⁵Cl}M+H]), 409.2 ([{³Cl}M+H]⁺). ESI pos.

e) ethyl 13-(2-chloro-6-fluoro-phenyl)-10-thioxo-7-thia-9,12-diazatricyclo[6.5.0.02,6]trideca-1(8), 2(6), 12-triene-4-carboxylate

A microwave vial was charged with ethyl 13-(2-chloro-6-fluoro-phenyl)-10-oxo-7-thia-9.12-diazatricyclo [6.5.0.02,6]trideca-1(8),2(6), 12-triene-4-carboxylate (2.57 g, 6.32 mmol). Lawesson's reagent (1.53 g, 3.79 mmol) and tetrahydrofuran (28.7 mL). The vial was capped and heated in a microwave oven to 100° C. for 20 min. The reaction mixture was concentrated in vacuo and the resulting crude was adsorbed on ISOLUTE® HM-N and purified by flash column chromatography (silica, 50 to 100% dichloromethane in heptane, 0 to 10% methanol in dichloromethane). The combined fractions were evaporated in vacuo then purified by flash column chromatography (silica, 0 to 10% methanol in dichloromethane) to afford the title compound (477 mg, 18%) as a red oil, containing ca. 15% of ethyl 13-(2-chloro-6-fluoro-phenyl)-10-thioxo-7-thia-9,12-diazatricyclo [6.5.0.02,6]trideca-1(8),2(6), 12-triene-5-carboxylate. MS: 423.1 ([{³⁵Cl}M+H]⁺). 425.0 ([{³⁷Cl}M+H]⁺). ESI pos.

f) ethyl 9-(2-chloro-6-fluoro-phenyl)-3-methyl-16-thia-2,4,5,8-tetrazatetracyclo [8.6.0.02,6.011.15]hexadeca-1(10),3,5,8,11(15)-pentaene-13-carboxylate

A microwave vial was charged with ethyl 13-(2-chloro-6-fluoro-phenyl)-10-thioxo-7-thia-9.12-diazatricyclo [6.5.0.02,6]trideca-1(8),2(6), 12-triene-4-carboxylate (450 mg, 1.06 mmol). acetohydrazide (101 mg, 1.36 mmol) and butan-1-ol (6.15 mL). The vial was capped and heated in a microwave oven to 130° C. for 55 min. The reaction mixture was poured into ethyl acetate and washed with water twice, dried (Na₂SO₄), filtered and concentrated in vacuo. The crude material was adsorbed on ISOLUTE® HM-N and purified by flash column chromatography (silica, 0 to 10% methanol in dichloromethane) to afford the title compound (155 mg, 33%) as a brown foam. containing ca. 15% of ethyl 9-(2-chloro-6-fluoro-phenyl)-3-methyl-16-thia-2,4,5,8-tetrazatetracyclo [8.6.0.02,6.011.15]hexadeca-1(10),3.5.8.11(15)-pentaene-14-carboxylate. MS: 445.1([{³⁵Cl}M+H]⁺), 447.1 ([{³⁷Cl}M+H]⁺). ESI pos.

g) 9-(2-chloro-6-fluoro-phenyl)-3-methyl-16-thia-2.4,5,8-tetrazatetracyclo [8.6.0.02,6.011,15]hexadeca-1(10),3,5,8,11(15)-pentaene-13-carboxylic acid

To a solution of ethyl 9-(2-chloro-6-fluoro-phenyl)-3-methyl-16-thia-2.4.5.8-tetrazatetracyclo [8.6.0.02,6.011.15]hexadeca-1(10),3.5.8.11(15)-pentaene-13-carboxylate (155 mg. 0.348 mmol) in methanol (2.96 mL) and water (0.148 mL) was added lithium hydroxide (29.2 mg. 1.22 mmol). The reaction mixture was stirred at 22° C. for 30 min, then concentrated in vacuo. diluted with water and the PH neutralized by addition of acetic acid (0.070 mL, 1.22 mmol). The suspension was diluted with dichloromethane and the phases were separated. The aqueous layer was extracted with further dichloromethane. The combined organic layers were dried (Na₂SO₄). filtered and concentrated in vacuo to afford the title compound (126 mg, 87%) as a brown oil. containing ca. 10% of 9-(2-chloro-6-fluoro-phenyl)-3-methyl-16-thia-2.4.5.8-tetrazatetracyclo [8.6.0.02,6.011.15]hexadeca-1(10),3.5.8.11(15)-pentaene-14-carboxy lic acid. MS: 417.1 ([{³⁵Cl}M+H]⁺), 419.1 ([{³⁷ Cl}M+H]⁺). ESI pos.

h) (13R)-9-(2-chloro-6-fluoro-phenyl)-3-methyl-16-thia-2.4,5,8-tetrazatetracyclo [8.6.0.02,6.011, 15]hexadeca-1(10),3,5.8.11(15)-pentaene-13-carboxamide

To a solution of 9-(2-chloro-6-fluoro-phenyl)-3-methyl-16-thia-2.4.5.8-tetrazatetracyclo [8.6.0.02,6.011.15]hexadeca-1(10),3.5.8.11(15)-pentaene-13-carboxy lic acid (127 mg, 0.305 mmol) in anhydrous pyridine (1.27 mL) was added di-tert-butyl dicarbonate (199 mg, 0.914 mmol) followed by ammonium bicarbonate (72.3 mg, 0.914 mmol). The mixture was stirred at 22° C, for 1.5 h. The reaction mixture was concentrated in vacuo, diluted with dichloromethane and methanol, adsorbed on ISOLUTE® HM-N and purified by flash column chromatography (silica, 0 to 20% methanol in dichloromethane), followed by chiral SFC (Chiralpak IG, 30% methanol) to afford the title compound (26 mg, 41%) as a light brown solid. MS: 416.2 ([{³⁵Cl}M+H]⁺), 418.2 ([{³⁷Cl}M+H]⁺), ESI pos.

i) (13R)-9-(2-chloro-6-fluoro-phenyl)-3-methyl-16-thia-2,4,5,8-tetrazatetracyclo [8.6.0.02,6.011,15]hexadeca-1(10),3,5,8, 11(15)-pentaene-13-carbonitrile

To a solution of (13R)-9-(2-chloro-6-fluoro-phenyl)-3-methyl-16-thia-2,4,5,8-tetrazatetracyclo [8.6.0.02,6.011, 15]hexadeca-1(10),3,5,8, 11(15)-pentaene-13-carboxamide (24 mg, 0.058 mmol) in anhydrous pyridine (0.288 mL) was added dropwise phosphoryl trichloride (10.6 mg, 6.45 μL, 69.3 umol) under nitrogen atmosphere. The mixture was stirred at 22° C. for 30 min. The reaction mixture was adsorbed on ISOLUTE® HM-N and purified by flash column chromatography (silica, 0 to 30% methanol in dichloromethane) to afford the (+)-title compound (11.3 mg, 49%) as a white solid. MS: 398.2 ([{³⁵Cl}M+H]⁺), 400.2 ([{³⁷Cl}M+H]⁺), ESI pos. 1H NMR (CDCl₃, 300 MHz) δ: 7.42-7.32 (m, 1H), 7.24 (br s, 1H), 7.10 (br d, J=3.4 Hz, 1H), 5.26-4.64 (m, 2H), 3.64-3.50 (m, 1H), 3.47-3.28 (m, 2H), 2.71 (s, 3H), 2.66-2.36 (m, 2H).

### Example 2

### (13R)-9-(2,6-difluorophenyl)-3-methyl-16-thia-2,4,5,8-tetrazatetracyclo [8.6.0.02,6.011,15]hexadeca-1(10),3,5,8,11(15)-pentaene-13-carbonitrile

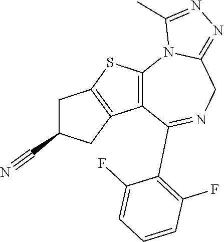 [SMILES: Cc1nnc2n1-c1sc3c(c1C(c1c(F)cccc1F)=NC2)C[C@@H](C#N)C3]

a) ethyl 2-amino-3-(2,6-difluorobenzoyl)-5,6-dihydro-4H-cyclopenta[b]thiophene-5-carboxylate

To a mixture of 3-(2,6-difluorophenyl)-3-oxo-propanenitrile (7.6 g, 42.0 mmol) and ethyl 3-oxocyclopentanecarboxylate (5.9 g, 37.8 mmol) in toluene (100 mL) was added □-alanine (0.67 g, 7.55 mmol) and acetic acid (7.54 mL, 131.8 mmol). The resulting yellow mixture was stirred at 110° C. (with a Dean-Stark apparatus) for 2 h, before being concentrated to 1/3 of the volume. The mixture was filtered directly through a sintered funnel and the filter cake was rinsed with toluene (150 mL). The clear yellow filtrate was concentrated in vacuo to obtain ethyl (E/Z)-3-[1-cyano-2-(2,6-difluorophenyl)-2-oxo-ethylidene]cyclopentanecarboxylate as a yellow viscous oil. The crude residue (16.3 g, 51.1 mmol) was dissolved in ethanol (65 mL) then sulfur (1.83 g, 57.2 mmol) and diisopropylamine (3.6 mL, 25.5 mmol) were added. The resulting yellow suspension was stirred at 70° C, for 3 h to form a red solution. The reaction mixture was concentrated in vacuo, then the resulting wet residue was suspended in tert-butyl methyl ether (250 mL) and stirred at room temperature for 15 min. The mixture was filtered over silica (100 g), washed with tert-butyl methyl ether (300 mL) and the filtrate concentrated in vacuo. The residue was purified by flash column chromatography (silica, 20% ethyl acetate in hexane) to afford the title compound (8.3 g, 62%) as a brown viscous oil, containing ca. 25% of ethyl 2-amino-3-(2,6-difluorobenzoyl)-5,6-dihydro-4H-cyclopenta [b]thiophene-6-carboxylate. MS: 352.2 ([M-H]⁻). ESI neg.

b) ethyl 2-[[2-(tert-butoxycarbonylamino) acetyl]amino]-3-(2,6-difluorobenzoyl)-5,6-dihydro-4H-cyclopenta [b]thiophene-5-carboxylate

To a mixture of (tert-butoxycarbonyl)glycine (14.5 g, 82.6 mmol) in 2-methyltetrahydrofuran (40 mL) was slowly added at a temperature between 0 to 5° C., 1,1′-carbonyldiimidazole (12.3 g, 76.0 mmol) in 2-methyltetrahydrofuran (60 mL). After 2 h, the mixture was allowed to warm to room temperature then a solution of ethyl 2-amino-3-(2,6-difluorobenzoyl)-5,6-dihydro-4H-cyclopenta [b]thiophene-5-carboxylate (11.6 g, 33.0 mmol) in 2-methyltetrahydrofuran (50 mL) was added, followed by Et3N (7.56 mL, 56.18 mmol). The resulting yellow solution was stirred at 90° C, for 6 h, before being diluted with tert-butyl methyl ether (70 mL). The organic phase was washed with aqueous citric acid (1.0 M, 350 mL), saturated aqueous sodium bicarbonate (300 mL) and brine (200 mL), then dried (Na₂SO₄) and concentrated in vacuo. The crude product was purified by flash column chromatography (silica, 40% ethyl acetate in hexane) to afford the title compound (11.9 g, 71%) as a light yellow solid, containing ca. 20% ethyl 2-[[2-(tert-butoxycarbonylamino)acetyl]amino]-3-(2,6-difluorobenzoyl)-5,6-dihydro-4H-cyclopenta [b]thiophene-6-carboxylate. MS: 526.2 ([M+H+NH3]⁺. ESI pos.

c) ethyl 2-[(2-aminoacetyl)amino]-3-(2,6-difluorobenzoyl)-5,6-dihydro-4H-cyclopenta [b]thiophene-5-carboxylate

To a solution of ethyl 2-[[2-(tert-butoxycarbonylamino)acetyl]amino]-3-(2,6-difluorobenzoyl)-5,6-dihydro-4H-cyclopenta[b]thiophene-5-carboxylate (5.0 g, 9.83 mmol) in dichloromethane (45 mL) was added 2,2,2-trifluoroacetic acid (7.6 mL, 98.3 mmol). The mixture was stirred at room temperature for 2.5 h, then concentrated in vacuo. The residue was dissolved in dichloromethane and washed with saturated aqueous sodium carbonate. The aqueous layer was extracted with dichloromethane. The combined organic layers were washed with brine, dried (Na₂SO₄), filtered and concentrated in vacuo to afford the title compound (4.0 g, 100%) as a yellow foam, containing ca. 20% of ethyl 2-[(2-aminoacetyl)amino]-3-(2,6-difluorobenzoyl)-5,6-dihydro-4H-cyclopenta [b]thiophene-6-carboxylate. MS: 409.2 ([M+H]⁺, ESI pos.

d) ethyl 13-(2,6-difluorophenyl)-10-oxo-7-thia-9,12-diazatricyclo[6.5.0.02,6]trideca-1(8),2(6), 12-triene-4-carboxylate

In analogy to experiment of example 1 d, ethyl 2-[(2-aminoacetyl)amino]-3-(2,6-difluorobenzoyl)-5,6-dihydro-4H-cyclopenta [b]thiophene-5-carboxylate was converted into the title compound (587 mg, 73%) which was obtained as a brown oil, containing ca. 15% of ethyl 13-(2,6-difluorophenyl)-10-oxo-7-thia-9,12-diazatricyclo [6.5.0.02,6]trideca-1(8), 2(6), 12-triene-5-carboxylate. MS: 389.3 ([M+H]⁺), ESI pos.

e) ethyl 13-(2,6-difluorophenyl)-10-thioxo-7-thia-9,12-diazatricyclo[6.5.0.02,6]trideca-1(8),2(6), 12-triene-4-carboxylate

In analogy to experiment of example 1 e, ethyl 13-(2,6-difluorophenyl)-10-oxo-7-thia-9,12-diazatricyclo [6.5.0.02,6]trideca-1(8),2(6), 12-triene-4-carboxylate was converted into the title compound (870 mg, 90%) which was obtained as a red foam, containing ca. 12% of ethyl 13-(2,6-difluorophenyl)-10-thioxo-7-thia-9,12-diazatricyclo [6.5.0.02,6]trideca-1(8),2(6), 12-triene-5-carboxylate. MS: 407.2 ([M+H]⁺), ESI pos.

f) ethyl 9-(2,6-difluorophenyl)-3-methyl-16-thia-2,4,5,8-tetrazatetracyclo [8.6.0.02,6.011,15]hexadeca-1(10),3,5,8, 11(15)-pentaene-13-carboxylate

In analogy to experiment of example 1 f, ethyl 13-(2,6-difluorophenyl)-10-thioxo-7-thia-9,12-diazatricyclo [6.5.0.02,6]trideca-1(8),2(6), 12-triene-4-carboxylate was converted into the title compound (100 mg, 35%) which was obtained as a brown oil, containing ca. 10% of ethyl 9-(2,6-difluorophenyl)-3-methyl-16-thia-2,4,5,8-tetrazatetracyclo [8.6.0.02,6.011,15]hexadeca-1(10), 3,5,8,11(15)-pentaene-14-carboxylate. MS: 429.3 ([M+H]⁺), ESI pos.

g) 9-(2,6-difluorophenyl)-3-methyl-16-thia-2,4,5,8-tetrazatetracyclo[8.6.0.02,6.011,15]hexadeca-1(10), 3,5,8,11(15)-pentaene-13-carboxylic acid

In analogy to experiment of example 1 g, ethyl 9-(2,6-difluorophenyl)-3-methyl-16-thia-2,4,5,8-tetrazatetracyclo [8.6.0.02,6.011, 15]hexadeca-1(10),3,5,8, 11(15)-pentaene-13-carboxylate was converted into the title compound (115 mg, 100%) which was obtained as a brown oil, containing ca. 20% of 9-(2,6-difluorophenyl)-3-methyl-16-thia-2,4,5,8-tetrazatetracyclo [8.6.0.02,6.011,15]hexadeca-1(10),3,5,8,11(15)-pentaene-14-carboxy lic acid MS: 401.2 ([M+H]⁺), ESI pos

h) (13R)-9-(2,6-difluorophenyl)-3-methyl-16-thia-2,4,5,8-tetrazatetracyclo [8.6.0.02,6.011, 15]hexadeca-1(10),3,5,8, 11(15)-pentaene-13-carboxamide

In analogy to experiment of example 1 h, 9-(2,6-difluorophenyl)-3-methyl-16-thia-2,4,5,8-tetrazatetracyclo [8.6.0.02,6.011, 15]hexadeca-1(10),3,5,8, 11(15)-pentaene-13-carboxylic acid was converted into the title compound (9 mg, 10%) which was obtained as an orange oil. MS: 400.3 ([M+H]⁺), ESI pos.

i) (13R)-9-(2,6-difluorophenyl)-3-methyl-16-thia-2,4,5,8-tetrazatetracyclo [8.6.0.02,6.011,15]hexadeca-1(10),3,5,8,11(15)-pentaene-13-carbonitrile

In analogy to experiment of example 1i, (13R)-9-(2,6-difluorophenyl)-3-methyl-16-thia-2,4,5,8-tetrazatetracyclo [8.6.0.02,6.011, 15]hexadeca-1(10),3,5,8, 11(15)-pentaene-13-carboxamide was converted into the title compound (7 mg, 78%) which was obtained as an orange oil. MS: 382.2 ([M+H]⁺), ESI pos. 1H NMR (CDCl₃, 300 MHZ) δ: 7.48-7.38 (m, 1H), 6.98 (t, J=8.4 Hz, 2H), 5.26-4.66 (m, 2H), 3.55 (d, J=8.1 Hz, 1H), 3.48-3.29 (m, 2H), 2.72 (s, 3H), 2.66-2.38 (m, 2H).

### Example 3

### (13R)-13-(difluoromethyl)-9-(2,6-difluorophenyl)-3-methyl-16-thia-2,4,5,8-tetrazatetracyclo [8.6.0.02,6.011,15]hexadeca-1(10),3,5,8,11(15)-pentaene

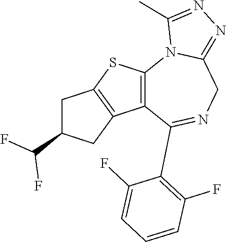 [SMILES: Cc1nnc2n1-c1sc3c(c1C(c1c(F)cccc1F)=NC2)C[C@@H](C(F)F)C3]

a) [9-(2,6-difluorophenyl)-3-methyl-16-thia-2,4,5,8-tetrazatetracyclo[8.6.0.02,6.011,15]hexadeca-1(10), 3,5,8,11(15)-pentaen-13-yl]methanol

To a solution of ethyl 9-(2,6-difluorophenyl)-3-methyl-16-thia-2,4,5,8-tetrazatetracyclo [8.6.0.02,6.011, 15]hexadeca-1(10), 3,5,8, 11(15)-pentaene-13-carboxylate (500 mg, 1.17 mmol) in 2-methylpropan-2-ol (5.8 mL) and methanol (1.2 mL) was added sodium borohydride (133 mg, 3.53 mmol) under argon. The mixture was stirred at 76° C. for 1 h, before being concentrated in vacuo. The residue was partitioned between water and dichloromethane, then the phases were separated. The aqueous layer was extracted with dichloromethane. The combined organic layers were washed with water, dried (Na₂SO₄), filtered and concentrated in vacuo to afford the title compound (440 mg, 98%) as a light brown foam, containing ca. 10% of [9-(2,6-difluorophenyl)-3-methyl-16-thia-2,4,5,8-tetrazatetracyclo [8.6.0.02,6.011, 15]hexadeca-1(10), 3,5,8,11(15)-pentaen-14-yl]methanol. MS: 387.2 ([M+H]⁺), ESI pos.

b) 9-(2,6-difluorophenyl)-3-methyl-16-thia-2,4,5,8-tetrazatetracyclo[8.6.0.02,6.011,15]hexadeca-1(10), 3,5,8,11(15)-pentaene-13-carbaldehyde

To a solution of [9-(2,6-difluorophenyl)-3-methyl-16-thia-2,4,5,8-tetrazatetracyclo [8.6.0.02,6.011, 15]hexadeca-1(10),3,5,8,11(15)-pentaen-13-yl]methanol (218 mg, 0.564 mmol) in dichloromethane (4.36 mL) was added Dess-Martin periodinane (359 mg, 0.846 mmol). The mixture was stirred at 22° C. for 2 h, before being quenched by addition of aqueous sodium carbonate (5 wt. %). The mixture was diluted with ethyl acetate then the phases separated. The aqueous layer was extracted with ethyl acetate twice. The combined organic layers were washed with aqueous sodium carbonate (5 wt. %) and brine, then dried (Na₂SO₄), filtered and concentrated in vacuo. The crude material was purified by flash column chromatography (silica, 0 to 5% methanol in dichloromethane) to afford the title compound (172 mg, 79%) as a brown oil, containing ca. 10% of 9-(2,6-difluorophenyl)-3-methyl-16-thia-2,4,5,8-tetrazatetracyclo [8.6.0.02,6.011, 15]hexadeca-1(10),3,5,8,11(15)-pentaene-14-carbaldehyde. MS: 385.2 ([M+H]⁺), ESI pos.

c) (13R)-13-(difluoromethyl)-9-(2,6-difluorophenyl)-3-methyl-16-thia-2,4,5,8-tetrazatetracyclo [8.6.0.02,6.011,15]hexadeca-1(10),3,5,8, 11(15)-pentaene

To a solution of triethylamine trihydrofluoride (64.2 mg, 0.390 mmol) in anhydrous dichloromethane (0.3 mL) at a temperature between 0 and 5° C., was added (diethy lamino)difluorosulfonium tetrafluoroborate (67 mg, 0.293 mmol) then a solution of 9-(2,6-difluorophenyl)-3-methyl-16-thia-2,4,5,8-tetrazatetracyclo [8.6.0.02,6.011,15]hexadeca-1(10), 3,5,8,11(15)-pentaene-13-carbaldehyde (75 mg, 0.195 mmol) in anhydrous dichloromethane (0.3 mL). The mixture was stirred at a temperature between 0 and 5° C. for 1 h, before being quenched by addition of aqueous sodium bicarbonate (5 wt. %). After vigorous stirring for 15 min, the phases were separated. The aqueous layer was extracted with dichloromethane twice. The combined organic layers were dried (Na₂SO₄), filtered and concentrated in vacuo. The crude material was purified by flash column chromatography (silica, 0 to 5% methanol in dichloromethane), followed by chiral HPLC (Chiralpak AD, heptane/0.1% ammonium acetate in ethanol, 6:4) to afford the (+)-title compound (4 mg, 5%) as a yellow oil. MS: 407.2 ([M+H]⁺), ESI pos. 1H NMR (CDCl₃, 400 MHZ) δ: 7.40 (tt, J=6.4, 8.4 Hz, 1H), 6.96 (t, J=8.4 Hz, 2H), 5.93-5.58 (m, 1H), 5.45-4.62 (m, 2H), 3.28-3.09 (m, 2H), 3.09-2.96 (m, 1H), 2.72 (s, 3H), 2.44-2.12 (m, 2H).

### Example 4

### (13R)-9-(2-chloro-6-fluoro-phenyl)-13-(difluoromethyl)-3-methyl-16-thia-2,4,5,8-tetrazatetracyclo [8.6.0.02,6.011,15]hexadeca-1(10),3,5,8,11(15)-pentaene

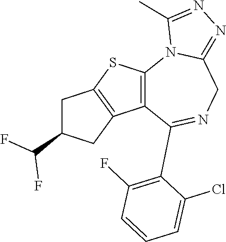 [SMILES: Cc1nnc2n1-c1sc3c(c1C(c1c(F)cccc1Cl)=NC2)C[C@@H](C(F)F)C3]

a) [9-(2-chloro-6-fluoro-phenyl)-3-methyl-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl 9-(2-chloro-6-fluoro-phenyl)-3-methyl-16-thia-2,4,5,8-tetrazatetracyclo [8.6.0.02,6.011, 15]hexadeca-1(10),3,5,8,11(15)-pentaene-13-carboxylate was converted into the title compound (300 mg, 97%) which was obtained as a light yellow solid, which was used as such in the following step without further characterization.

b) 9-(2-chloro-6-fluoro-phenyl)-3-methyl-16-thia-2,4,5,8-tetrazatetracyclo [8.6.0.02,6.011,15]hexadeca-1(10),3,5,8,11(15)-pentaene-13-carbaldehyde

In analogy to experiment of example 3 b, [9-(2-chloro-6-fluoro-phenyl)-3-methyl-16-thia-2,4,5,8-tetrazatetracyclo [8.6.0.02,6.011, 15]hexadeca-1(10),3,5,8, 11(15)-pentaen-13-yl]methanol was converted into the title compound (148 mg, 74%) which was obtained as a light yellow solid. MS: 400.9 ([{³⁵Cl}M+H]⁺), 402.9 ([{³⁷Cl}M+H]⁺), ESI pos.

c) (13R)-9-(2-chloro-6-fluoro-phenyl)-13-(difluoromethyl)-3-methyl-16-thia-2,4,5,8-tetrazatetracyclo [8.6.0.02,6.011,15]hexadeca-1(10),3,5,8,11(15)-pentaene

In analogy to experiment of example 3 c, 9-(2-chloro-6-fluoro-phenyl)-3-methyl-16-thia-2,4,5,8-tetrazatetracyclo [8.6.0.02,6.011, 15]hexadeca-1(10),3,5,8, 11(15)-pentaene-13-carbaldehyde was converted into the (+)-title compound (21 mg, 32%) which was obtained as a light pink solid. MS: 423.2 ([{³⁵Cl}M+H]⁺), 425.2 ([{³⁷Cl}M+H]⁺), ESI pos. 1H NMR (CDCl₃, 400 MHZ) δ: 7.36 (dt, J=5.9, 8.3 Hz, 1H), 7.26-7.21 (m, 1H), 7.13-7.04 (m, 1H), 5.96-5.55 (m, 1H), 5.26-4.64 (m, 2H), 3.23-3.09 (m, 2H), 3.08-2.99 (m, 1H), 2.71 (s, 3H), 2.44-2.07 (m, 2H).

### Example 5

### (7S,13R)-13-(difluoromethyl)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene

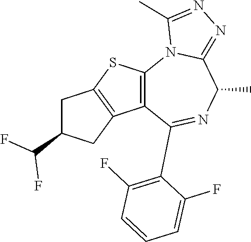 [SMILES: Cc1nnc2n1-c1sc3c(c1C(c1c(F)cccc1F)=N[C@H]2C)C[C@@H](C(F)F)C3]

a) ethyl 2-[[(2S)-2-(tert-butoxycarbonylamino)propanoyl]amino]-3-(2,6-difluorobenzoyl)-5,6-dihydro-4H-cyclopenta [b]thiophene-5-carboxylate

To a solution of (2S)-2-(tert-butoxycarbony lamino)propanoic acid (264 mg, 1.39 mmol) in N,N-dimethylformamide (7.15 mL) was added 2-(1H-benzotriazole-1-yl)-1, 1,3,3-tetramethy laminium tetrafluoroborate (493 mg, 1.53 mmol) and N,N-diisopropylethy lamine (1.22 mL, 6.97 mmol). The mixture was stirred at room temperature for 10 min, before addition of a solution of ethyl 2-amino-3-(2,6-difluorobenzoyl)-5,6-dihydro-4H-cyclopenta [b]thiophene-5-carboxylate (500 mg, 1.39 mmol) in N.N-dimethylformamide (4.77 mL). The reaction mixture was stirred for 18 h, before being concentrated in vacuo by rotary evaporation. The residue was diluted with ethyl acetate (10 mL) then aqueous sodium hydroxide (1.0 M, 5 mL) was added and the mixture was stirred at room temperature for 5 min. The phases were separated. The aqueous layer was extracted with ethyl acetate (3×15 mL). The combined organic layers were dried (Na₂SO₄), filtered and concentrated in vacuo. The crude material was adsorbed on ISOLUTE® HM-N and purified by flash column chromatography (silica, 0 to 30% ethyl acetate in heptane) to afford the title compound (534 mg, 73%) as a yellow solid, containing ca. 25% of ethyl 2-[[(2S)-2-(tert-butoxycarbonylamino)propanoyl]amino]-3-(2,6-difluorobenzoyl)-5,6-dihydro-4H-cyclopenta [b]thiophene-6-carboxylate. MS: 521.4 ([M-H]⁻), ESI neg.

b) ethyl 2-[[(2S)-2-aminopropanoyl]amino]-3-(2,6-difluorobenzoyl)-5,6-dihydro-4H-cyclopenta [b]thiophene-5-carboxylate

In analogy to experiment of example 2 c, ethyl 2-[[(2S)-2-(tert-butoxycarbonylamino)propanoyl]amino]-3-(2,6-difluorobenzoyl)-5,6-dihydro-4H-cyclopenta [b]thiophene-5-carboxylate was converted into the title compound (578 mg, 94%) which was obtained as a brown oil, containing ca. 25% of ethyl 2-[[(2S)-2-aminopropanoyl]amino]-3-(2,6-difluorobenzoyl)-5,6-dihydro-4H-cyclopenta[b]thiophene-6-carboxylate. MS: 423.3 ([M+H]⁺), ESI pos.

c) ethyl (11S)-13-(2,6-difluorophenyl)-11-methyl-10-oxo-7-thia-9,12-diazatricyclo [6.5.0.02,6]trideca-1(8),2(6), 12-triene-4-carboxylate

In analogy to experiment of example 1 d, ethyl 2-[[(2S)-2-aminopropanoyl]amino]-3-(2,6-difluorobenzoyl)-5,6-dihydro-4H-cyclopenta [b]thiophene-5-carboxylate was converted into the title compound (464 mg, 83%) which was obtained as a brown oil, containing ca. 25% of ethyl (11S)-13-(2,6-difluorophenyl)-11-methyl-10-oxo-7-thia-9,12-diazatricyclo[6.5.0.02,6]trideca-1(8), 2(6), 12-triene-5-carboxylate. MS: 405.2 ([M+H]⁺), ESI pos.

d) ethyl (11S)-13-(2,6-difluorophenyl)-11-methyl-10-thioxo-7-thia-9,12-diazatricyclo [6.5.0.02,6]trideca-1(8),2(6), 12-triene-4-carboxylate

In analogy to experiment of example 1 e, ethyl (11S)-13-(2,6-difluorophenyl)-11-methyl-10-oxo-7-thia-9,12-diazatricyclo [6.5.0.02,6]trideca-1(8),2(6), 12-triene-4-carboxylate was converted into the title compound (148 mg, 35%) which was obtained as a brown solid, containing ca. 25% of ethyl (11S)-13-(2,6-difluorophenyl)-11-methyl-10-thioxo-7-thia-9,12-diazatricyclo[6.5.0.02,6]trideca-1(8), 2(6), 12-triene-5-carboxylate. MS: 421.2 ([M+H]⁺), ESI pos.

e) ethyl (7S)-9-(2,6-difluorophenyl)-3,7-dimethyl-16-thia-2,4,5,8-tetrazatetracyclo [8.6.0.02,6.011,15]hexadeca-1(10),3.5.8.11(15)-pentaene-13-carboxylate

In analogy to experiment of example 1 f, ethyl 13-(2,6-difluorophenyl)-10-thioxo-7-thia-9,12-diazatricyclo [6.5.0.02,6]trideca-1(8),2(6), 12-triene-4-carboxylate was converted into the title compound (34 mg, 21%) which was obtained as a brown solid, containing ca. 25% of ethyl (7S)-9-(2,6-difluorophenyl)-3,7-dimethyl-16-thia-2,4,5,8-tetrazatetracyclo [8.6.0.02,6.011,15]hexadeca-1(10), 3,5,8,11(15)-pentaene-14-carboxylate. MS: 443.2 ([M+H]⁺), ESI pos.

f) [(7S)-9-(2,6-difluorophenyl)-3,7-dimethyl-16-thia-2,4,5,8-tetrazatetracyclo [8.6.0.02,6.011.15]hexadeca-1(10),3.5.8.11(15)-pentaen-13-yl]methanol

In analogy to experiment of example 3a, ethyl (7S)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene-13-carboxylate was converted into the title compound (2.8 g, 77%) which was obtained as a yellow solid, containing ca. 25% of [(7S)-9-(2,6-difluorophenyl)-3,7-dimethyl-16-thia-2,4,5,8-tetrazatetracyclo [8.6.0.02,6.011, 15]hexadeca-1(10),3,5,8, 11(15)-pentaen-14-yl]methanol. MS: 401.1 ([M+H]⁺), ESI pos.

g (7S)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene-13-carbaldehyde

In analogy to experiment of example 3 b, [(7S)-9-(2,6-difluorophenyl)-3,7-dimethyl-16-thia-2,4,5,8-tetrazatetracyclo [8.6.0.02,6.011, 15]hexadeca-1(10),3,5,8,11(15)-pentaen-13-yl]methanol was converted into the title compound (4 g, 53%) which was obtained as a yellow solid, containing ca. 25% of (7S)-9-(2,6-difluorophenyl)-3,7-dimethyl-16-thia-2,4,5,8-tetrazatetracyclo [8.6.0.02,6.011, 15]hexadeca-1(10),3,5,8,11(15)-pentaene-14-carbaldehyde. MS: 399.2 ([M+H]⁺), ESI pos.

h) (7S,13R)-13-(difluoromethyl)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene

In analogy to experiment of example 3 c, (7S)-9-(2,6-difluorophenyl)-3,7-dimethyl-16-thia-2,4,5,8-tetrazatetracyclo [8.6.0.02,6.011, 15]hexadeca-1(10),3,5,8,11(15)-pentaene-13-carbaldehyde was converted into the (-)-title compound (271 mg, 23%) which was obtained as a white solid. MS: 420.8 ([M+H]⁺), ESI pos. 1H NMR (CDCl₃, 400 MHZ) δ: 7.38 (tt, J=6.3, 8.4 Hz, 1H), 7.07-6.82 (m, 2H), 5.96-5.60 (m, 1H), 4.41 (br s, 1H), 3.23-2.98 (m, 3H), 2.71 (s, 3H), 2.52 (br d, J=13.8 Hz, 1H), 2.18-2.02 (m, 4H).

### Example 6

### (13R)-9-(2-chloro-6-fluoro-phenyl)-3-cyclopropyl-13-(difluoromethyl)-16-thia-2,4,5,8-tetrazatetracyclo [8.6.0.02,6.011,15]hexadeca-1(10),3,5,8,11(15)-pentaene

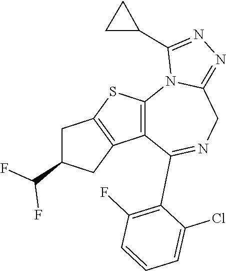 [SMILES: Fc1cccc(Cl)c1C1=NCc2nnc(C3CC3)n2-c2sc3c(c21)C[C@@H](C(F)F)C3]

a) ethyl (10E.Z)-13-(2-chloro-6-fluoro-phenyl)-10-hydrazinylidene-7-thia-9,12-diazatricyclo [6.5.0.02,6]trideca-1(8),2(6), 12-triene-4-carboxylate

To a solution of ethyl 13-(2-chloro-6-fluoro-phenyl)-10-oxo-7-thia-9,12-diazatricyclo [6.5.0.02,6]trideca-1(8),2(6), 12-triene-4-carboxylate (1.3 g, 3.07 mmol) in 2-propanol (4 mL) was added hydrazine hydrate (308 mg, 6.15 mmol). The mixture was stirred at 25° C. for 1 h, before being concentrated in vacuo by rotary evaporation. The residue was purified by flash column chromatography (silica, ethyl acetate/petroleum ether 10 to 80%) to afford the title compound (310 mg, 21%) as dark brown gum. MS: 421.2 ([M+H]⁺), 423.2 ([{³⁷Cl}M+H]⁺), ESI pos.

b) ethyl 9-(2-chloro-6-fluoro-phenyl)-3-cyclopropyl-16-thia-2,4,5,8-tetrazatetracyclo [8.6.0.02,6.011,15]hexadeca-1(10),3,5,8, 11(15)-pentaene-13-carboxylate

To a mixture of ethyl (10E/Z)-13-(2-chloro-6-fluoro-phenyl)-10-hydrazinylidene-7-thia-9,12-diazatricyclo [6.5.0.02,6]trideca-1(8),2(6), 12-triene-4-carboxylate (295 mg, 0.70 mmol) in pyridine (5 mL) was added cyclopropanecarbonyl chloride (80.6 mg, 0.77 mmol) at 15° C. The mixture was stirred at 15° C. for 30 min and then at 110° C. for 4 h. The mixture was cooled to room temperature before being concentrated in vacuo. The residue was dissolved in ethyl acetate and purified by flash column chromatography (silica, methanol/ethyl acetate 5 to 15%) to afford the title compound (270 mg, 40%) as a light brown gum. MS: 471.2 ([M+H]⁺), 473.2 ([{³⁷Cl}M+H]⁺), ESI pos.

c) [9-(2-chloro-6-fluoro-phenyl)-3-cyclopropyl-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl 9-(2-chloro-6-fluoro-phenyl)-3-cyclopropyl-16-thia-2,4,5,8-tetrazatetracyclo [8.6.0.02,6.011, 15]hexadeca-1(10),3,5,8,11(15)-pentaene-13-carboxylate was converted into the title compound (160 mg, 35%) which was obtained as a light yellow solid. MS: 429.1 ([M+H]⁺), 431.1 ([{³⁷Cl}M+H]⁺), ESI pos.

d) 9-(2-chloro-6-fluoro-phenyl)-3-cyclopropyl-16-thia-2,4,5,8-tetrazatetracyclo [8.6.0.02,6.011,15]hexadeca-1(10),3,5,8,11(15)-pentaene-13-carbaldehyde

In analogy to experiment of example 3 b, [9-(2-chloro-6-fluoro-phenyl)-3-cyclopropyl-16-thia-2,4,5,8-tetrazatetracyclo [8.6.0.02,6.011, 15]hexadeca-1(10),3,5,8,11(15)-pentaen-13-yl]methanol was converted into the title compound (80 mg, 56%) which was obtained as a light yellow solid. MS: 427.3 ([M+H]⁺), 429.3 ([{³⁷Cl}M+H]⁺), ESI pos.

e) (13R)-9-(2-chloro-6-fluoro-phenyl)-3-cyclopropyl-13-(difluoromethyl)-16-thia-2,4,5,8-tetrazatetracyclo [8.6.0.02,6.011,15]hexadeca-1(10),3,5,8,11(15)-pentaene

In analogy to experiment of example 3 c, 9-(2-chloro-6-fluoro-phenyl)-3-cyclopropyl-16-thia-2,4,5,8-tetrazatetracyclo [8.6.0.02,6.011, 15]hexadeca-1(10),3,5,8,11(15)-pentaene-13-carbaldehyde was converted into the (+)-title compound (8 mg, 15%) which was obtained as a white solid. MS:

449.2 ([M+H]⁺), 451.2 ([{³⁷Cl}M+H]⁺), ESI pos. 1H NMR (CDCl₃, 400 MHZ) δ: 7.39-7.32 (m, 1H), 7.26-7.21 (m, 1H), 7.12-7.05 (m, 1H), 5.96-5.56 (m, 1H), 5.36-4.70 (m, 2H), 3.26-3.08 (m, 2H), 3.08-2.99 (m, 1H), 2.46-2.11 (m, 2H), 2.10-2.02 (m, 1H), 1.28-1.25 (m, 2H), 1.20-1.13 (m, 2H).

### Example 7

### (13R)-3-cyclopropyl-13-(difluoromethyl)-9-(2,6-difluorophenyl)-16-thia-2,4,5,8-tetrazatetracyclo [8.6.0.02,6.011,15]hexadeca-1(10),3,5,8,11(15)-pentaene

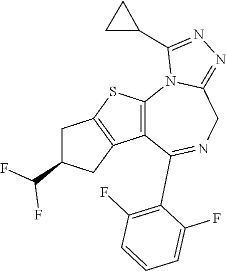 [SMILES: Fc1cccc(F)c1C1=NCc2nnc(C3CC3)n2-c2sc3c(c21)C[C@@H](C(F)F)C3]

a) ethyl 3-cyclopropyl-9-(2,6-difluorophenyl)-16-thia-2,4,5,8-tetrazatetracyclo [8.6.0.02,6.011,15]hexadeca-1(10),3,5,8,11(15)-pentaene-13-carboxylate

In analogy to experiment of example 1 f, ethyl 13-(2,6-difluorophenyl)-10-thioxo-7-thia-9,12-diazatricyclo [6.5.0.02,6]trideca-1(8),2(6), 12-triene-4-carboxylate using cyclopropanecarbohydrazide was converted into the title compound (200 mg, 18%) which was obtained as a light yellow solid. MS: 455.0 ([M+H]⁺), ESI pos.

b) [3-cyclopropyl-9-(2,6-difluorophenyl)-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl 3-cyclopropyl-9-(2,6-difluorophenyl)-16-thia-2,4,5,8-tetrazatetracyclo [8.6.0.02,6.011, 15]hexadeca-1(10),3,5,8,11(15)-pentaene-13-carboxylate was converted into the title compound (165 mg, 91%) which was obtained as a light yellow oil. MS: 413.0 ([M+H]⁺), ESI pos.

c) 3-cyclopropyl-9-(2,6-difluorophenyl)-16-thia-2,4,5,8-tetrazatetracyclo [8.6.0.02,6.011,15]hexadeca-1(10),3.5.8.11(15)-pentaene-13-carbaldehyde

In analogy to experiment of example 3 b, [3-cyclopropyl-9-(2,6-difluorophenyl)-16-thia-2,4,5,8-tetrazatetracyclo [8.6.0.02,6.011, 15]hexadeca-1(10),3,5,8, 11(15)-pentaen-13-yl]methanol was converted into the title compound (100 mg, 61%) which was obtained as a light yellow solid. MS: 411.0 ([M+H]⁺), ESI pos.

d) (13R)-3-cyclopropyl-13-(difluoromethyl)-9-(2,6-difluorophenyl)-16-thia-2,4,5,8-tetrazatetracyclo [8.6.0.02,6.011,15]hexadeca-1(10),3,5,8,11(15)-pentaene

In analogy to experiment of example 3 c, 3-cyclopropyl-9-(2,6-difluorophenyl)-16-thia-2,4,5,8-tetrazatetracyclo [8.6.0.02,6.011, 15]hexadeca-1(10),3,5,8, 11(15)-pentaene-13-carbaldehyde was converted into the (+)-title compound (9 mg, 8%) which was obtained as a light red solid. MS: 433.0 ([M+H]⁺), ESI pos. 1H NMR (CDCl₃, 400 MHZ) δ: 7.40 (tt, J=6.3, 8.5 Hz, 1H), 6.96 (t, J=8.4 Hz, 2H), 5.95-5.57 (m, 1H), 5.43-4.65 (m, 2H), 3.27-3.10 (m, 2H), 3.08-2.94 (m, 1H), 2.43-2.14 (m, 2H), 2.08 (tt, J=5.1, 8.2 Hz, 1H), 1.26 (br s, 2H), 1.19-1.13 (m, 2H).

### Example 8

### (7S,13R)-3-cyclopropyl-13-(difluoromethyl)-9-(2,6-difluorophenyl)-7-methyl-16-thia-2,4,5,8-tetrazatetracyclo [8.6.0.02,6.011,15]hexadeca-1(10),3,5,8,11(15)-pentaene

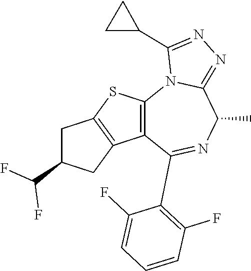 [SMILES: C[C@@H]1N=C(c2c(F)cccc2F)c2c(sc3c2C[C@@H](C(F)F)C3)-n2c(C3CC3)nnc21]

a) ethyl (7S)-3-cyclopropyl-9-(2,6-difluorophenyl)-7-methyl-16-thia-2,4,5,8-tetrazatetracyclo [8.6.0.02,6.011,15]hexadeca-1(10),3.5.8.11(15)-pentaene-13-carboxylate

In analogy to experiment of example 1 f, ethyl 13-(2,6-difluorophenyl)-10-thioxo-7-thia-9,12-diazatricyclo [6.5.0.02,6]trideca-1(8),2(6), 12-triene-4-carboxylate using cyclopropane carbohydrazide was converted into the title compound (1.4 g, 70%) which was obtained as a yellow solid. MS: 469.2 ([M+H]⁺), ESI pos.

b) (7S)-3-cyclopropyl-9-(2,6-difluorophenyl)-7-methyl-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl (7S)-3-cyclopropyl-9-(2,6-difluorophenyl)-7-methyl-16-thia-2,4,5,8-tetrazatetracyclo [8.6.0.02,6.011, 15]hexadeca-1(10),3,5,8,11(15)-pentaene-13-carboxylate was converted into the title compound (1.1 g, 93%) which was obtained as a yellow solid. MS: 427.2 ([M+H]⁺), ESI pos.

c) (7S)-3-cyclopropyl-9-(2,6-difluorophenyl)-7-methyl-16-thia-2,4,5,8-tetrazatetracyclo [8.6.0.02,6.011,15]hexadeca-1(10),3,5,8,11(15)-pentaene-13-carbaldehyde

In analogy to experiment of example 3 b, (7S)-3-cyclopropyl-9-(2,6-difluorophenyl)-7-methyl-16-thia-2,4,5,8-tetrazatetracyclo [8.6.0.02,6.011,15]hexadeca-1(10),3,5,8, 11(15)-pentaen-13-yl]methanol was converted into the title compound (870 mg, 87%) which was obtained as a brown solid. MS: 425.1 ([M+H]⁺), ESI pos.

d) (7S,13R)-3-cyclopropyl-13-(difluoromethyl)-9-(2,6-difluorophenyl)-7-methyl-16-thia-2,4,5,8-tetrazatetracyclo [8.6.0.02,6.011,15]hexadeca-1(10),3,5,8, 11(15)-pentaene

In analogy to experiment of example 3 c, (7S)-3-cyclopropyl-9-(2,6-difluorophenyl)-7-methyl-16-thia-2,4,5,8-tetrazatetracyclo [8.6.0.02,6.011,15]hexadeca-1(10),3,5,8,11(15)-pentaene-13-carbaldehyde was converted into the (-)-title compound (126 mg, 14%) which was obtained as a white solid. MS: 447.1 ([M+H]⁺), ESI pos. 1H NMR (CDCl₃, 400 MHZ) δ: 7.38 (tt, J=8.44, 6.37 Hz, 1 H) 6.76-7.11 (m, 2 H) 5.57-5.96 (m, 1 H) 4.41 (br s, 1 H) 2.96-3.24 (m, 3 H) 2.53 (br d, J=16.69 Hz, 1 H) 1.95-2.19 (m, 5 H) 1.29-1.42 (m, 1 H) 1.03-1.24 (m, 3 H).

### Example 9

### (7S,13R)-9-(2-chloro-6-fluoro-phenyl)-13-(difluoromethyl)-3,7-dimethyl-16-thia-2,4,5,8-tetrazatetracyclo [8.6.0.02,6.011,15]hexadeca-1(10),3,5,8,11(15)-pentaene

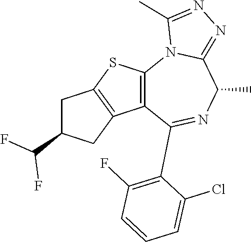 [SMILES: Cc1nnc2n1-c1sc3c(c1C(c1c(F)cccc1Cl)=N[C@H]2C)C[C@@H](C(F)F)C3]

a) ethyl 2-[[(2S)-2-(tert-butoxycarbonylamino)propanoyl]amino]-3-(2-chloro-6-fluoro-benzoyl)-5,6-dihydro-4H-cyclopenta[b]thiophene-5-carboxylate

In analogy to experiment of example 2 b, ethyl 2-amino-3-(2-chloro-6-fluoro-benzoyl)-5,6-dihydro-4H-cyclopenta [b]thiophene-5-carboxylate using (2S)-2-(tert-butoxycarbonylamino)propanoic acid was converted into the title compound (28.7 g, 65%) which was obtained as a yellow solid. MS: 483.2 ([{³⁵Cl}M-C₄H8+H]⁺), ESI pos.

b) ethyl 2-[[(2S)-2-aminopropanoyl]amino]-3-(2-chloro-6-fluoro-benzoyl)-5,6-dihydro-4H-cyclopenta [b]thiophene-5-carboxylate

In analogy to experiment of example 2 c, ethyl 2-[[(2S)-2-(tert-butoxycarbonylamino)propanoyl]amino]-3-(2-chloro-6-fluoro-benzoyl)-5,6-dihydro-4H-cyclopenta[b]thiophene-5-carboxylate was converted into the title compound (25 g, 100%) which was obtained as a yellow solid. MS: 439.2 ([{³⁵Cl}M+H]⁺), ESI pos.

c) ethyl (11S)-13-(2-chloro-6-fluoro-phenyl)-11-methyl-10-oxo-7-thia-9,12-diazatricyclo [6.5.0.02,6]trideca-1(8),2(6), 12-triene-4-carboxylate

In analogy to experiment of example 1 d, ethyl 2-[[(2S)-2-aminopropanoyl]amino]-3-(2-chloro-6-fluoro-benzoyl)-5,6-dihydro-4H-cyclopenta[b]thiophene-5-carboxylate was converted into the title compound (8.3 g, 24%) which was obtained as a yellow solid. MS: 421.2 ([{³⁵Cl}M+H]⁺), ESI pos.

d) ethyl (11S)-13-(2-chloro-6-fluoro-phenyl)-11-methyl-10-thioxo-7-thia-9,12-diazatricyclo [6.5.0.02,6]trideca-1(8),2(6), 12-triene-4-carboxylate

In analogy to experiment of example 1 e, ethyl (11S)-13-(2-chloro-6-fluoro-phenyl)-11-methyl-10-oxo-7-thia-9, 12-diazatricyclo [6.5.0.02,6]trideca-1(8),2(6), 12-triene-4-carboxylate was converted into the title compound (1.2 g, 100%) which was obtained as a red foam. MS: 437.2 ([{³⁵Cl}M+H]⁺), ESI pos.

e) ethyl (10E/Z,11S)-13-(2-chloro-6-fluoro-phenyl)-10-hydrazinylidene-11-methyl-7-thia-9,12-diazatricyclo [6.5.0.02,6]trideca-1(8),2(6), 12-triene-4-carboxylate

In analogy to experiment of example 6 a, ethyl (11S)-13-(2-chloro-6-fluoro-phenyl)-11-methyl-10-thioxo-7-thia-9,12-diazatricyclo [6.5.0.02,6]trideca-1(8),2(6), 12-triene-4-carboxylate was converted into the title compound (300 mg, 31%) which was obtained as an orange oil. MS: 435.2 ([{³⁵Cl}M+H]⁺). ESI pos.

f) ethyl (7S)-9-(2-chloro-6-fluoro-phenyl)-3.7-dimethyl-16-thia-2,4,5,8-tetrazatetracyclo [8.6.0.02,6.011,15]hexadeca-1(10),3,5.8.11(15)-pentaene-13-carboxylate

To a solution of ethyl (10E/Z,11S)-13-(2-chloro-6-fluoro-phenyl)-10-hydrazinylidene-11-methyl-7-thia-9,12-diazatricyclo [6.5.0.02,6]trideca-1(8),2(6), 12-triene-4-carboxylate (510 mg, 1.17 mmol) in toluene (10 mL) was added trimethylorthoacetate (282 mg, 2.35 mmol). The mixture was stirred at 110° C, for 2 h. The reaction mixture was allowed to cool to room temperature then concentrated in vacuo. The residue was purified by flash column chromatography (silica, ethyl acetate/methanol 3 to 18%) to afford the title compound (285 mg, 53%) as a yellow oil. MS: 459.2 ([{³⁵Cl}M+H]⁺), ESI pos.

g) [(7S)-9-(2-chloro-6-fluoro-phenyl)-3.7-dimethyl-16-thia-2,4,5,8-tetrazatetracyclo [8.6.0.02,6.011, 15]hexadeca-1(10),3.5.8.11(15)-pentaen-13-yl]methanol

In analogy to experiment of example 3 a, ethyl (7S)-9-(2-chloro-6-fluoro-phenyl)-3,7-dimethyl-16-thia-2,4,5,8-tetrazatetracyclo [8.6.0.02,6.011, 15]hexadeca-1(10),3,5,8, 11(15)-pentaene-13-carboxylate was converted into the title compound (180 mg, 70%) which was obtained as a yellow solid. MS: 417.2 ([{³⁵Cl}M+H]⁺), ESI pos.

h) [(7S)-9-(2-chloro-6-fluoro-phenyl)-3,7-dimethyl-16-thia-2,4,5,8-tetrazatetracyclo [8.6.0.02,6.011.15]hexadeca-1(10),3.5,8,11(15)-pentaen-13-yl]methanol

In analogy to experiment of example 3 b. [(7S)-9-(2-chloro-6-fluoro-phenyl)-3,7-dimethyl-16-thia-2,4,5,8-tetrazatetracyclo [8.6.0.02,6.011, 15]hexadeca-1(10),3.5,8,11(15)-pentaen-13-yl]methanol was converted into the title compound (150 mg, 84%) which was obtained as a yellow solid. MS: 415.2 ([{³⁵Cl}M+H]⁺), ESI pos.

i) (75.13R)-9-(2-chloro-6-fluoro-phenyl)-13-(difluoromethyl)-3.7-dimethyl-16-thia-2,4,5,8-tetrazatetracyclo [8.6.0.02,6.011,15]hexadeca-1(10),3,5,8,11(15)-pentaene

In analogy to experiment of example 3 c. [(7S)-9-(2-chloro-6-fluoro-phenyl)-3,7-dimethyl-16-thia-2,4,5,8-tetrazatetracyclo [8.6.0.02,6.011, 15]hexadeca-1(10),3,5,8,11(15)-pentaen-13-yl]methanol was converted into the (-)-title compound (12 mg, 7%) which was obtained as a white solid. MS: 437.2 ([{³⁵Cl}M+H]⁺), ESI pos. 1H NMR (CDCl₃, 400 MHZ) δ: 7.37-7.31 (m, 1H), 7.19-7.12 (m, 1H), 7.01-6.90 (m, 1H), 5.98-5.62 (m, 1H), 4.47 (s, 1H), 3.20-3.09 (m, 2H), 3.05 (t. J=10.2 Hz, 1H), 2.76-2.66 (m, 3H), 2.60 (d, J=15.7 Hz, 1H), 2.13 (d, J=6.7 Hz, 3H), 2.04 (dd, J=8.4, 15.6 Hz, 1H).

### Example 10

### (13R)-13-(difluoromethyl)-9-(2,6-difluorophenyl)-3-pyridazin-3-yl-16-thia-2,4,5,8-tetrazatetracyclo [8.6.0.02,6.011,15]hexadeca-1(10),3,5,8,11(15)-pentaene

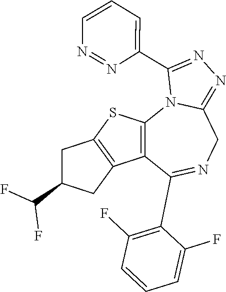 [SMILES: Fc1cccc(F)c1C1=NCc2nnc(-c3cccnn3)n2-c2sc3c(c21)C[C@@H](C(F)F)C3]

a) ethyl 9-(2,6-difluorophenyl)-3-pyridazin-3-yl-16-thia-2,4,5,8-tetrazatetracyclo [8.6.0.02,6.011,15]hexadeca-1(10),3,5,8,11(15)-pentaene-13-carboxylate

In analogy to experiment of example 1 f, ethyl 13-(2,6-difluorophenyl)-10-thioxo-7-thia-9,12-diazatricyclo [6.5.0.02,6]trideca-1(8),2(6), 12-triene-4-carboxylate using pyridazine-3-carbohydrazide was converted into the title compound (310 mg, 45%) which was obtained as a red solid. MS: 493.0 ([{³⁵Cl}M+H]⁺), ESI pos.

b) [9-(2,6-difluorophenyl)-3-pyridazin-3-yl-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl 9-(2,6-difluorophenyl)-3-pyridazin-3-yl-16-thia-2,4,5,8-tetrazatetracyclo [8.6.0.02,6.011, 15]hexadeca-1(10),3,5,8,11(15)-pentaene-13-carboxylate was converted into the title compound (100 mg, 38%) which was obtained as a light yellow solid. MS: 451.0 ([{³⁵Cl}M+H]⁺), ESI pos.

c) 9-(2,6-difluorophenyl)-3-pyridazin-3-yl-16-thia-2,4,5,8-tetrazatetracyclo [8.6.0.02,6.011,15]hexadeca-1(10),3,5,8,11(15)-pentaene-13-carbaldehyde

In analogy to experiment of example 3 b, [9-(2,6-difluorophenyl)-3-pyridazin-3-yl-16-thia-2,4,5,8-tetrazatetracyclo [8.6.0.02,6.011, 15]hexadeca-1(10),3,5,8, 11(15)-pentaen-13-yl]methanol was converted into the title compound (90 mg, 86%) which was obtained as a light yellow solid. MS: 449.0 ([{³⁵Cl}M+H]⁺), ESI pos.

d) (13R)-13-(difluoromethyl)-9-(2,6-difluorophenyl)-3-pyridazin-3-yl-16-thia-2,4,5,8-tetrazatetracyclo [8.6.0.02,6.011,15]hexadeca-1(10),3,5,8,11(15)-pentaene

In analogy to experiment of example 3 c, 9-(2,6-difluorophenyl)-3-pyridazin-3-yl-16-thia-2,4,5,8-tetrazatetracyclo [8.6.0.02,6.011, 15]hexadeca-1(10),3,5,8, 11(15)-pentaene-13-carbaldehyde was converted into the (+)-title compound (4 mg, 4%) which was obtained as an off-white solid. MS: 471.0 ([{³⁵Cl}M+H]⁺), ESI pos. 1H NMR (CDCl₃, 400 MHZ) δ: 9.30 (dd, J=1.6, 5.1 Hz, 1H), 8.33 (dd, J=1.8, 8.5 Hz, 1H), 7.70 (dd, J=5.0, 8.5 Hz, 1H), 7.40 (tt, J=6.4, 8.4 Hz, 1H), 7.03-6.89 (m, 2H), 5.91-5.58 (m, 1H), 3.24-3.05 (m, 2H), 3.03-2.91 (m, 1H), 2.44-2.18 (m, 2H).

### Example 11

### (7S,13R)-9-(2-chloro-6-fluoro-phenyl)-3-cyclopropyl-13-(difluoromethyl)-7-methyl-16-thia-2,4,5,8-tetrazatetracyclo [8.6.0.02,6.011,15]hexadeca-1(10),3,5,8,11(15)-pentaene

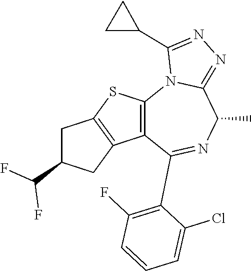 [SMILES: C[C@@H]1N=C(c2c(F)cccc2Cl)c2c(sc3c2C[C@@H](C(F)F)C3)-n2c(C3CC3)nnc21]

a) ethyl (7S)-9-(2-chloro-6-fluoro-phenyl)-3-cyclopropyl-7-methyl-16-thia-2,4,5,8-tetrazatetracyclo [8.6.0.02,6.011,15]hexadeca-1(10),3,5,8,11(15)-pentaene-13-carboxylate

In analogy to experiment of example 6 b, ethyl (10E Z)-13-(2-chloro-6-fluoro-phenyl)-10-hydrazinylidene-7-thia-9,12-diazatricyclo [6.5.0.02,6]trideca-1(8),2(6), 12-triene-4-carboxylate was converted into the title compound (330 mg, 71%) which was obtained as a brown solid. MS: 485.2 ([{³⁵Cl}+H]⁺), ESI pos.

b) [(7S)-9-(2-chloro-6-fluoro-phenyl)-3-cyclopropyl-7-methyl-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl (7S)-9-(2-chloro-6-fluoro-phenyl)-3-cyclopropyl-7-methyl-16-thia-2,4,5,8-tetrazatetracyclo [8.6.0.02,6.011, 15]hexadeca-1(10),3,5,8,11(15)-pentaene-13-carboxylate was converted into the title compound (200 mg, 66%) which was obtained as a brown gum, which was used as such in the following step without further characterization.

c) (7S)-9-(2-chloro-6-fluoro-phenyl)-3-cyclopropyl-7-methyl-16-thia-2,4,5,8-tetrazatetracyclo [8.6.0.02,6.011,15]hexadeca-1(10),3,5,8,11(15)-pentaene-13-carbaldehyde

In analogy to experiment of example 3 b, [(7S)-9-(2-chloro-6-fluoro-phenyl)-3-cyclopropyl-7-methyl-16-thia-2,4,5,8-tetrazatetracyclo [8.6.0.02,6.011, 15]hexadeca-1(10),3,5,8,11(15)-pentaen-13-yl]methanol was converted into the title compound (199 mg, 100%) which was obtained as a brown gum. MS: 441.1 ([{³⁵Cl}M+H]⁺), ESI pos.

d) (75,13R)-9-(2-chloro-6-fluoro-phenyl)-3-cyclopropyl-13-(difluoromethyl)-7-methyl-16-thia-2,4,5,8-tetrazatetracyclo [8.6.0.02,6.011,15]hexadeca-1(10),3,5,8,11(15)-pentaene

In analogy to experiment of example 3 c, (7S)-9-(2-chloro-6-fluoro-phenyl)-3-cyclopropyl-7-methyl-16-thia-2,4,5,8-tetrazatetracyclo [8.6.0.02,6.011, 15]hexadeca-1(10),3,5,8,11(15)-pentaene-13-carbaldehyde was converted into the (-)-title compound (13 mg, 7%) which was obtained as a white solid. MS: 462.8 ([{³⁵Cl}M+H]⁺), ESI pos. 1H NMR (CDCl₃, 400 MHZ) δ: 7.36-7.32 (m, 1H), 7.17-7.12 (m, 1H), 7.01-6.92 (m, 1H), 5.97-5.61 (m, 1H), 4.53-4.36 (m, 1H), 3.22-3.01 (m, 3H), 2.66-2.54 (m, 1H), 2.14-2.08 (m, 3H), 2.07-1.99 (m, 2H), 1.33-1.30 (m, 1H), 1.24-1.18 (m, 3H).

### Example 12

### (7S,13R)-9-(2-chloro-6-fluoro-phenyl)-13-(difluoromethyl)-7-methyl-3-pyridazin-3-yl-16-thia-2,4,5,8-tetrazatetracyclo [8.6.0.02,6.011,15]hexadeca-1(10),3,5,8,11(15)-pentaene

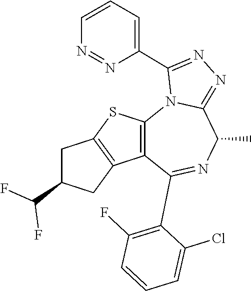 [SMILES: C[C@@H]1N=C(c2c(F)cccc2Cl)c2c(sc3c2C[C@@H](C(F)F)C3)-n2c(-c3cccnn3)nnc21]

a) ethyl (7S)-9-(2-chloro-6-fluoro-phenyl)-7-methyl-3-pyridazin-3-yl-16-thia-2,4,5,8-tetrazatetracyclo [8.6.0.02,6.011,15]hexadeca-1(10),3,5,8,11(15)-pentaene-13-carboxylate

In analogy to experiment of example 1 f, ethyl 13-(2-chloro-6-fluoro-phenyl)-10-thioxo-7-thia-9,12-diazatricyclo [6.5.0.02,6]trideca-1(8),2(6), 12-triene-4-carboxylate using pyridazine-3-carbohydrazide was converted into the title compound (500 mg, 43%) which was obtained as a light yellow gum. MS: 523.0 ([{³⁵Cl}M+H]⁺), ESI pos.

b) [(7S)-9-(2-chloro-6-fluoro-phenyl)-7-methyl-3-pyridazin-3-yl-16-thia-2,4,5,8-tetrazatetracyclo [8.6.0.02,6.011,15]hexadeca-1(10),3,5,8,11(15)-pentaen-13-yl]methanol

In analogy to experiment of example 3 a, ethyl (7S)-9-(2-chloro-6-fluoro-phenyl)-7-methyl-3-pyridazin-3-yl-16-thia-2,4,5,8-tetrazatetracyclo [8.6.0.02,6.011,15]hexadeca-1(10),3,5,8,11(15)-pentaene-13-carboxylate was converted into the title compound (358 mg, 78%) which was obtained as a light yellow gum. MS: 481.1 ([{³⁵Cl}M+H]⁺), ESI pos.

c) (7S)-9-(2-chloro-6-fluoro-phenyl)-7-methyl-3-pyridazin-3-yl-16-thia-2,4,5,8-tetrazatetracyclo [8.6.0.02,6.011, 15]hexadeca-1(10),3,5,8,11(15)-pentaene-13-carbaldehyde

In analogy to experiment of example 3 b, [(7S)-9-(2-chloro-6-fluoro-phenyl)-7-methyl-3-pyridazin-3-yl-16-thia-2,4,5,8-tetrazatetracyclo [8.6.0.02,6.011,15]hexadeca-1(10),3,5,8,11(15)-pentaen-13-yl]methanol was converted into the title compound (300 mg, 92%) which was obtained as an off-white solid. MS: 479.1 ([{³⁵Cl}M+H]⁺), ESI pos.

d) (7S,13R)-9-(2-chloro-6-fluoro-phenyl)-13-(difluoromethyl)-7-methyl-3-pyridazin-3-yl-16-thia-2,4,5,8-tetrazatetracyclo [8.6.0.02,6.011,15]hexadeca-1(10),3,5,8,11(15)-pentaene

In analogy to experiment of example 3 c, (7S)-9-(2-chloro-6-fluoro-phenyl)-7-methyl-3-pyridazin-3-yl-16-thia-2,4,5,8-tetrazatetracyclo [8.6.0.02,6.011,15]hexadeca-1(10),3,5,8,11(15)-pentaene-13-carbaldehyde was converted into the (-)-title compound (6 mg, 9%) which was obtained as an off-white solid. MS: 501.2 ([{³⁵Cl}M+H]⁺), ESI pos. 1H NMR (CDCl₃, 400 MHZ) : 9.31-9.25 (m, 1H), 8.34-8.28 (m, 1H), 7.74-7.65 (m, 1H), 7.35-7.30 (m, 1H), 7.17-7.09 (m, 1H), 7.03-6.86 (m, 1H), 6.05-5.58 (m, 1H), 4.64-4.36 (m, 1H), 3.18-3.01 (m, 2H), 3.00-2.92 (m, 1H), 2.70-2.60 (m, 1H), 2.25-2.16 (m, 3H), 2.14-2.02 (m, 1H).

### Example 13

### (7S,13R)-9-(2,6-difluorophenyl)-3,7-dimethyl-13-(trifluoromethyl)-16-thia-2,4,5,8-tetrazatetracyclo [8.6.0.02,6.011,15]hexadeca-1(10),3,5,8,11(15)-pentaene

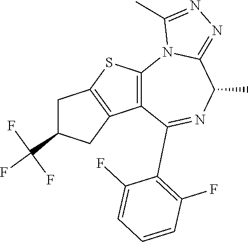 [SMILES: Cc1nnc2n1-c1sc3c(c1C(c1c(F)cccc1F)=N[C@H]2C)C[C@@H](C(F)(F)F)C3]

a) [2-amino-5-(trifluoromethyl)-5,6-dihydro-4H-cyclopenta[b]thiophen-3-yl]-(2,6-difluorophenyl)methanone

In analogy to experiment of example 1 a, 3-(2,6-difluorophenyl)-3-oxo-propanenitrile using 3-(trifluoromethyl)cyclopentanone instead of ethyl 3-oxocyclopentanecarboxylate was converted into the title compound (990 mg, 85%) which was obtained as a yellow foam, containing ca. 20% of [2-amino-6-(trifluoromethyl)-5,6-dihydro-4H-cyclopenta[b]thiophen-3-yl](2,6-difluorophenyl)methanone. MS: 348.2 ([M+H]⁺), ESI pos.

b) ethyl 2-[[(2S)-2-(tert-butoxycarbony lamino)propanoyl]amino]-3-(2,6-difluorobenzoyl)-5,6-dihydro-4H-cyclopenta[b]thiophene-5-carboxylate

In analogy to experiment of example 5 a, [2-amino-5-(trifluoromethyl)-5,6-dihydro-4H-cyclopenta [b]thiophen-3-yl]-(2,6-difluorophenyl)methanone was purified by SFC separation (Column: achiral 2-ethylpyridine, 5% methanol) to obtain the title compound (1.19 g, 81%) as a yellow waxy solid and its regioisomer ethyl 2-[[(2S)-2-(tert-butoxycarbonylamino)propanoyl]amino]-3-(2,6-difluorobenzoyl)-5,6-dihydro-4H-cyclopenta[b]thiophene-6-carboxylate (104 mg, 5%) as a yellow solid. MS: 517.4 ([M+H]⁺), ESI pos and MS: 517.4 ([M+H]⁺), ESI pos, respectively.

c) (2S)-2-amino-N-[3-(2,6-difluorobenzoyl)-5-(trifluoromethyl)-5,6-dihydro-4H-cyclopenta [b]thiophen-2-yl]propanamide

In analogy to experiment of example 2 c, ethyl 2-[[(2S)-2-(tert-butoxycarbonylamino)propanoyl]amino]-3-(2,6-difluorobenzoyl)-5,6-dihydro-4H-cyclopenta [b]thiophene-5-carboxylate was converted into the title compound (791 mg, 76%) which was obtained as a yellow solid. MS: 419.2 ([M+H]⁺), ESI pos.

d) (11S)-13-(2,6-difluorophenyl)-11-methyl-4-(trifluoromethyl)-7-thia-9,12-diazatricyclo [6.5.0.02,6]trideca-1(8),2(6), 12-trien-10-one

In analogy to experiment of example 1 d, (2S)-2-amino-N-[3-(2,6-difluorobenzoyl)-5-(trifluoromethyl)-5,6-dihydro-4H-cyclopenta [b]thiophen-2-yl]propanamide was converted into the title compound (451 mg, 36%) which was obtained as an orange solid. MS: 401.2 ([M+H]⁺), ESI pos.

e) (11S)-13-(2,6-difluorophenyl)-11-methyl-4-(trifluoromethyl)-7-thia-9,12-diazatricyclo [6.5.0.02,6]trideca-1(8),2(6), 12-triene-10-thione

In analogy to experiment of example 1 e, (11S)-13-(2,6-difluorophenyl)-11-methyl-4-(trifluoromethyl)-7-thia-9, 12-diazatricyclo [6.5.0.02,6]trideca-1(8),2(6), 12-trien-10-one was converted into the title compound (181 mg, 65%) which was obtained as an orange solid. MS: 417.2 ([M+H]⁺), ESI pos.

f) (7S)-9-(2,6-difluorophenyl)-3,7-dimethyl-13-(trifluoromethyl)-16-thia-2,4,5,8-tetrazatetracyclo [8.6.0.02,6.011,15]hexadeca-1(10),3,5,8,11(15)-pentaene

In analogy to experiment of example 1 f, (11S)-13-(2,6-difluorophenyl)-11-methyl-4-(trifluoromethyl)-7-thia-9,12-diazatricyclo [6.5.0.02,6]trideca-1(8),2(6), 12-triene-10-thione was converted into the title compound (91 mg, 48%) which was obtained as an orange solid. MS: 439.2 ([M+H]⁺), ESI pos.

g) (7S,13R)-9-(2,6-difluorophenyl)-3,7-dimethyl-13-(trifluoromethyl)-16-thia-2,4,5,8-tetrazatetracyclo [8.6.0.02,6.011,15]hexadeca-1(10),3,5,8,11(15)-pentaene

(7S)-9-(2,6-difluorophenyl)-3,7-dimethyl-13-(trifluoromethyl)-16-thia-2,4,5,8-tetrazatetracyclo [8.6.0.02,6.011, 15]hexadeca-1(10),3,5,8, 11(15)-pentaene (91 mg, 0.208 mmol) was purified by chiral HPLC (Reprosil Chiral-NR, 60% heptane/40% ethanol containing 20% ammonium acetate), followed by SFC (IH, 20 to 40% methanol) to obtain the (-)-title compound (21 mg, 24%) as a colorless oil. MS: 439.2 ([M+H]⁺), ESI pos. 1H NMR (CDCl₃, 300 MHz) δ: 7.39 (tt, J=6.3, 8.5 Hz, 1H), 7.04-6.83 (m, 2H), 4.43 (br d, J=5.8 Hz, 1H), 3.50-3.29 (m, 1H), 3.44-3.09 (m, 2H), 2.72-2.62 (m, 1H), 2.70 (s, 3H), 2.18-2.07 (m, 4H).

### Example 14

### (13R)-9-(2,6-difluorophenyl)-3,13-dimethyl-16-thia-2,4,5,8-tetrazatetracyclo [8.6.0.02,6.011,15]hexadeca-1(10),3,5,8,11(15)-pentaene

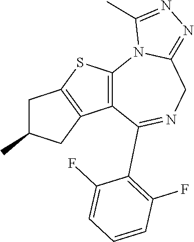 [SMILES: Cc1nnc2n1-c1sc3c(c1C(c1c(F)cccc1F)=NC2)C[C@@H](C)C3]

a) [9-(2,6-difluorophenyl)-3-methyl-16-thia-2,4,5,8-tetrazatetracyclo[8.6.0.02,6.011,15]hexadeca-1(10), 3,5,8,11(15)-pentaen-13-yl]methyl methanesulfonate

To a solution of [9-(2,6-difluorophenyl)-3-methyl-16-thia-2,4,5,8-tetrazatetracyclo [8.6.0.02,6.011, 15]hexadeca-1(10),3,5,8, 11(15)-pentaen-13-yl]methanol (85 mg, 0.220 mmol) in dichloromethane (2.2 mL) was added Et;N (92 μl, 660 mmol) and methanesulfonyl chloride (34 μl, 0.440 mmol). The mixture was stirred at room temperature for 16 h, before addition of water. The phases were separated and the aqueous layer was extracted with dichloromethane (3×10 mL). The combined organic layers were washed with aqueous sodium carbonate (5 wt. %, 10 mL), dried (Na₂SO₄), filtered and concentrated in vacuo to afford the title compound (91 mg, 83%) as a brown solid. MS: 465.1 ([M+H]⁺), ESI pos.

b) 9-(2,6-difluorophenyl)-13-(iodomethyl)-3-methyl-16-thia-2,4,5,8-tetrazatetracyclo [8.6.0.02,6.011,15]hexadeca-1(10),3,5,8,11(15)-pentaene

To a solution of [9-(2,6-difluorophenyl)-3-methyl-16-thia-2,4,5,8-tetrazatetracyclo [8.6.0.02,6.011, 15]hexadeca-1(10), 3,5,8, 11(15)-pentaen-13-yl]methyl methanesulfonate (91 mg, 0.196 mmol) in acetone (1.96 mL) was added LiI (524 mg, 3.92 mmol). The mixture was stirred at room temperature for 64 h, before addition of saturated aqueous sodium sulfite. The phases were separated and the aqueous layer was extracted with dichloromethane (3×10 mL). The combined organic layers were dried (Na₂SO₄), filtered and concentrated in vacuo to afford the title compound (138 mg, 97%) as a brown solid. MS: 497.1 ([M+H]⁺), ESI pos.

c) 9-(2,6-difluorophenyl)-3,13-dimethyl-16-thia-2,4,5,8-tetrazatetracyclo [8.6.0.02,6.011,15]hexadeca-1(10),3,5,8,11(15)-pentaene

To a solution of 9-(2,6-difluorophenyl)-13-(iodomethyl)-3-methyl-16-thia-2,4,5,8-tetrazatetracyclo [8.6.0.02,6.011,15]hexadeca-1(10),3,5,8,11(15)-pentaene (138 mg, 0.192 mmol) and Et₃N (54 μl, 0.384 mmol) in methanol (1.6 mL) was added Pd/C (20.4 mg, 0.192 mmol). The mixture was stirred under hydrogen atmosphere at room temperature for 41 h then filtered through a pad of dicalite. The filter cake was rinsed with methanol and the filtrate was concentrated in vacuo. The residue was taken with dichloromethane (10 mL) and washed with aqueous hydrochloric acid (1.0 M, 2 × 10 mL). The organic layers were dried (Na₂SO₄), filtered and concentrated in vacuo. The crude material was adsorbed on ISOLUTE® HM-N and purified by flash column chromatography (silica, 0 to 10% methanol in dichloromethane) to afford the title compound (43 mg, 55%) as a brown solid. MS: 371.1 ([M+H]⁺), ESI pos.

d) (13R)-9-(2,6-difluorophenyl)-3,13-dimethyl-16-thia-2,4,5,8-tetrazatetracyclo [8.6.0.02,6.011,15]hexadeca-1(10),3,5,8,11(15)-pentaene

9-(2,6-difluorophenyl)-3,13-dimethyl-16-thia-2,4,5,8-tetrazatetracyclo [8.6.0.02,6.011, 15]hexadeca-1(10),3,5,8, 11(15)-pentaene (43 mg, 0.117 mmol) was purified by HPLC (Chiralpak AD, 80% heptane/20% ethanol containing 20% ammonium acetate) to afford the (+)-title compound (8 mg, 18%) as a white solid. MS: 371.1 ([M+H]⁺), ESI pos. 1H NMR (CDCl₃, 300 MHz) : 7.37 (tt, J=6.3, 8.5 Hz, 1H), 6.93 (t, J=8.4 Hz, 2H), 5.30 (s, 1H), 5.26-4.55 (m, 2H), 3.14-3.04 (m, 1H), 2.86-2.76 (m, 1H), 2.71 (s, 3H), 2.53 (tdd, J=1.7, 6.1, 15.5 Hz, 1H), 2.41-2.09 (m, 1H), 1.89-1.64 (m, 1H).

### Example 15

### (7S,13R)-9-(2,6-difluorophenyl)-3,7,13-trimethyl-16-thia-2,4,5,8-tetrazatetracyclo [8.6.0.02,6.011,15]hexadeca-1(10),3,5,8,11(15)-pentaene

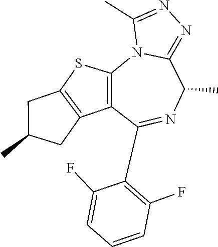

a) (2-amino-5-methyl-5,6-dihydro-4H-cyclopenta[b]thiophen-3-yl)-(2,6-difluorophenyl)methanone

In analogy to experiment of example 1 a, 3-(2,6-difluorophenyl)-3-oxo-propanenitrile using 3-methylcyclopentanone instead of ethyl 3-oxocyclopentanecarboxylate was converted into the title compound (4.88 g, 60%) which was obtained as a yellow foam, containing ca. 45% of (2-amino-6-methyl-5,6-dihydro-4H-cyclopenta[b]thiophen-3-yl)-(2,6-difluorophenyl)methanone. MS: 294.1 ([M+H]⁺), ESI pos.

b) tert-butyl N-[(1S)-2-[[3-(2,6-difluorobenzoyl)-5-methyl-5,6-dihydro-4H-cyclopenta[b]thiophen-2-yl]amino]-1-methyl-2-oxo-ethyl]carbamate

In analogy to experiment of example 5 a. (2-amino-5-methyl-5,6-dihydro-4H-cyclopenta [b]thiophen-3-yl)-(2,6-difluorophenyl)methanone was converted into the title compound (7.1 g, 87%) which was obtained as a yellow solid, containing ca. 45% of tert-butyl N-[(1S)-2-[[3-(2,6-difluorobenzoyl)-6-methyl-5,6-dihydro-4H-cyclopenta [b]thiophen-2-yl]amino]-1-methyl-2-oxo-ethyl]carbamate. MS: 464.5 ([M+H]⁺). ESI pos.

c) (2S)-2-amino-N-[3-(2,6-difluorobenzoyl)-5-methyl-5,6-dihydro-4H-cyclopenta[b]thiophen-2-yl]propanamide

To a solution of tert-butyl N-[(1S)-2-[[3-(2,6-difluorobenzoyl)-5-methyl-5,6-dihydro-4H-cyclopenta [b]thiophen-2-yl]amino]-1-methyl-2-oxo-ethyl]carbamate (8.18 g, 17.6 mmol) in dichloromethane (127 mL) at a temperature between 0 and 5° C., was added hydrochloric acid (4.0 M in 1.4-dioxane, 44 mL, 176 mmol). The mixture was stirred between 0 and 5° C. for 10 min. before being allowed to warm to room temperature. After 6 h, the pH was adjusted to about 8 by addition of saturated aqueous sodium bicarbonate. The phases were separated. The aqueous layer was extracted with dichloromethane (2×300 mL). The combined organic layers were washed with brine (2×100 mL), dried (Na₂SO₄), filtered and concentrated in vacuo to afford the title compound (7.4 g, 113%) as a light brown oil, containing ca. 45% of (2S)-2-amino-N-[3-(2,6-difluorobenzoyl)-6-methyl-5,6-dihydro-4H-cyclopenta [b]thiophen-2-yl]propanamide and ca. 30% 1.4-dioxane. MS: 364.4 ([M+H]⁺). ESI pos.

d) benzyl N-[(1S)-2-[[3-(2,6-difluorobenzoyl)-5-methyl-5,6-dihydro-4H-cyclopenta[b]thiophen-2-yl]amino]-1-methyl-2-oxo-ethyl]carbamate

To a solution of (2S)-2-amino-N-[3-(2,6-difluorobenzoyl)-5-methyl-5,6-dihydro-4H-cyclopenta [b]thiophen-2-yl]propanamide (3.03 g, 5.82 mmol) in dichloromethane (42 mL) was added N,N-diisopropylethy lamine (972 mg, 1.32 mL, 7.57 mmol) and benzyl chloroformate (1.19 g, 1.0 mL, 7.01 mmol). The reaction mixture was stirred at room temperature for 30 min then aqueous hydrochloric acid (1.0 M, 25 mL) was added. The phases were separated and the aqueous layer was extracted with dichloromethane (3×25 mL). The combined organic layers were dried (Na₂SO₄), filtered and concentrated in vacuo. The residue was purified y flash column chromatography (silica, 10 to 70% ethyl acetate in heptane) to afford the title compound (1.62 g 55%) as a yellow solid, containing ca. 45% of benzyl N-[(1S)-2-[[3-(2,6-difluorobenzoyl)-6-methyl-5,6-dihydro-4H-cyclopenta[b]thiophen-2-yl]amino]-1-methyl-2-oxo-ethyl]carbamate. MS: 499.2 ([M+H]⁺). ESI pos.

e) benzyl N-[(1S)-2-[[(5R)-3-(2,6-difluorobenzoyl)-5-methyl-5,6-dihydro-4H-cyclopenta [b]thiophen-2-yl]amino]-1-methyl-2-oxo-ethyl]carbamate

benzyl N-[(1S)-2-[[3-(2,6-difluorobenzoyl)-5-methyl-5,6-dihydro-4H-cyclopenta[b]thiophen-2-yl]amino]-1-methyl-2-oxo-ethyl]carbamate (1.49 g, 2.98 mmol) was purified by SFC (chiral AD-H, 15% methanol/ethanol/isopropanol 1:1:1), followed by SFC (OZ-H, 35% methanol) to afford the title compound (230 mg, 15%) as a yellow solid. MS: 499.2 ([M+H]⁺). ESI pos.

f) (25)-2-amino-N-[(5R)-3-(2,6-difluorobenzoyl)-5-methyl-5,6-dihydro-4H-cyclopenta[b]thiophen-2-yl]propanamide

To a solution of benzyl N-[(1S)-2-[[(5R)-3-(2,6-difluorobenzoyl)-5-methyl-5,6-dihydro-4H-cyclopenta [b]thiophen-2-yl]amino]-1-methyl-2-oxo-ethyl]carbamate (120 mg, 0.241 mmol) in acetonitrile (2.4 mL) was added iodotrimethylsilane (82 □L, 0.602 mmol). The mixture was stirred at room temperature for 45 min, then HCl (4.0 M in 1.4-dioxane, 241 □L, 0.963 mmol) and methanol (108 □L, 2.67 mmol) were added. The solvent was concentrated in vacuo and the residue was dissolved in dichloromethane and diethyl ether and charged on a dropping funnel. The solution was added dropwise to heptane (75 mL) and the resulting precipitate was collected on a sintered funnel. The solid was dissolved in dichloromethane and methanol and washed with saturated aqueous sodium carbonate (15 mL). The aqueous layer was extracted with dichloromethane (2×50 mL). The combined organic layers were dried (Na₂SO₄), filtered and concentrated in vacuo to afford the title compound (84 mg, 95%) as a yellow solid. MS: 365.2 ([M+H]'). ESI pos.

g) (4R,11S)-13-(2,6-difluorophenyl)-4,11-dimethyl-7-thia-9,12-diazatricyclo[6.5.0.02,6]trideca-1(8), 2(6), 12-trien-10-one

In analogy to experiment of example 1 d. (25)-2-amino-N-[(5R)-3-(2,6-difluorobenzoyl)-5-methyl-5,6-dihydro-4H-cyclopenta[b]thiophen-2-yl]propanamide was converted into the title compound (80 mg, 81%) which was obtained as an orange solid. MS: 347.2 ([M+H]⁺). ESI pos.

h) (75,13R)-9-(2,6-difluorophenyl)-3.7.13-trimethyl-16-thia-2,4,5,8-tetrazatetracyclo [8.6.0.02,6.011, 15]hexadeca-1(10),3,5,8, 11(15)-pentaene

To a solution of (4R,11S)-13-(2,6-difluorophenyl)-4.11-dimethyl-7-thia-9.12-diazatricyclo [6.5.0.02,6]trideca-1(8),2(6), 12-trien-10-one (75 mg, 0.217 mmol) in anhydrous tetrahydrofuran (1.9 mL) at −70° C. was added potassium tert-butoxide (1.0 M in tetrahydrofuran, 0.32 mL, 0.325 mmol). The mixture was stirred for 5 min at −70° C, then allowed to warm up to −40° C. After 5 min, diethyl chlorophosphate (62 μL, 0.433 mmol) was added and the mixture was stirred at −40° C. for 5 min, then at 0° C. for 1 h. After this time, a solution of acetohydrazide (52 mg, 0.704 mmol) in isopropanol (0.26 mL, 3.46 mmol) was added. The mixture was allowed to warm up to room temperature and stirred for 1 h, then at 40° C. for 16 h. The reaction mixture was diluted with dichloromethane (10 mL), then washed with saturated aqueous sodium bicarbonate (5 mL). The aqueous phase was extracted with dichloromethane (2×25 mL). The combined organic phases were dried (Na₂SO₄) and concentrated in vacuo. The residue was purified with flash column chromatography (silica, dichloromethane/methanol, 0 to 10%), followed by chiral SFC (1H, 15% methanol) to afford the (-)-title compound (17 mg, 34%) as a light brown solid. MS: 385.2 ([M+H]⁺), ESI pos. 1H NMR (CDCl₃, 300 MHz) δ: 7.40-7.29 (m, 1H), 7.07-6.77 (m, 3H), 4.62-4.18 (m, 1H), 3.15-3.05 (m, 1H), 2.83-2.70 (m, 2H), 2.70 (s, 3H), 2.52 (tdd, J=1.5, 5.4, 15.7

Hz, 2H), 2.18-2.01 (m, 7H), 1.26 (s, 1H), 1.13 (d, J=6.9 Hz, 4H).

### Example 16 and Example 17

### (7S,13R)-13-(difluoromethyl)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene

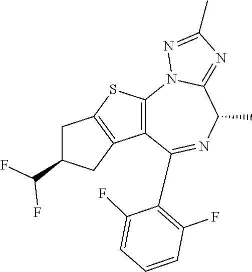 [SMILES: Cc1nc2n(n1)-c1sc3c(c1C(c1c(F)cccc1F)=N[C@H]2C)C[C@@H](C(F)F)C3]

(7S,13S)-13-(difluoromethyl)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene

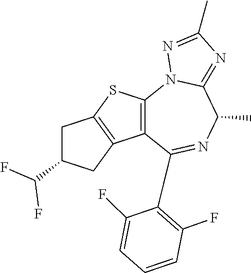 [SMILES: Cc1nc2n(n1)-c1sc3c(c1C(c1c(F)cccc1F)=N[C@H]2C)C[C@H](C(F)F)C3]

a) ethyl (11S)-9-amino-13-(2,6-difluorophenyl)-11-methyl-10-oxo-7-thia-9,12-diazatricyclo [6.5.0.02,6]trideca-1(8),2(6), 12-triene-4-carboxylate

To a solution of ethyl (11S)-13-(2,6-difluorophenyl)-11-methyl-10-oxo-7-thia-9,12-diazatricyclo [6.5.0.02,6]trideca-1(8),2(6), 12-triene-4-carboxylate (20 mg, 43 umol) in N,N-dimethylformamide (0.43 mL) at a temperature between 0 and 5° C., was added O-(diphenylphosphinyl)hydroxylamine (12 mg, 52 umol) and cesium carbonate (21 mg, 64.5 umol). The suspension was stirred at 0° C. for 3 h, before being diluted with water (30 mL) and ethyl acetate (50 mL). The phases were separated, then the aqueous layer was extracted with ethyl acetate (3×50 mL). The combined organic layers were dried (Na₂SO₄), filtered and concentrated in vacuo. The residue was purified by flash column chromatography (silica, 0% to 100% ethyl acetate in heptane) to afford the title compound (10 mg, 53%) as a light yellow solid. MS: 420.2 ([M+H]⁺), ESI pos.

b) ethyl (7S)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene-13-carboxylate

To a solution of ethyl (11S)-9-amino-13-(2,6-difluorophenyl)-11-methyl-10-oxo-7-thia-9,12-diazatricyclo [6.5.0.02,6]trideca-1(8),2(6), 12-triene-4-carboxylate (155 mg, 0.370 mmol) in anhydrous pyridine (3.7 mL) was added ethyl acetimidate hydrochloride (320 mg, 2.59 mmol). The mixture was stirred at room temperature for 16 h, then heated to 60° C. for 2 h. The reaction mixture was absorbed over ISOLUTE® HM-N and purified by flash column chromatography (silica, dichloromethane/methanol, 0% to 10%) to afford the title compound (68 mg, 41%) as a white solid. MS: 443.3 ([M+H]⁺), ESI pos.

c) [(7S)-9-(2,6-difluorophenyl)-4,7-dimethyl-16-thia-2,3,5,8-tetrazatetracyclo [8.6.0.02,6.011,15]hexadeca-1(10),3.5.8.11(15)-pentaen-13-yl]methanol

In analogy to experiment of example 3 a, ethyl (7S)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene-13-carboxylate was converted into the title compound (450 mg, 60%) which was obtained as a brown oil. MS:

401.1 ([M+H]⁺), ESI pos.

d) (7S)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene-13-carbaldehyde

In analogy to experiment of example 3 b. [(7S)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaen-13-yl]methanol was converted into the title compound (74 mg, 18%) which was obtained as a brown solid. MS: 399.1 ([{³⁵Cl}M+H]⁺), ESI pos.

e) (7S)-13-(difluoromethyl)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene

In analogy to experiment of example 3 c. (7S)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene-13-carbaldehyde was converted into the title compound (74 mg, 51%) which was obtained as a brown solid. MS: 421.5 ([{³⁵Cl}M+H]⁺), ESI pos.

f) (7S,13R)-13-(difluoromethyl)-9-(2,6-difluorophenyl)-4.7-dimethyl-16-thia-2,3,5,8-tetrazatetracyclo [8.6.0.02,6.011,15]hexadeca-1(10),3,5,8,11(15)-pentaene

(7S,13S)-13-(difluoromethyl)-9-(2,6-difluorophenyl)-4,7-dimethyl-16-thia-2,3,5,8-tetrazatetracyclo [8.6.0.02,6.011,15]hexadeca-1(10),3,5,8,11(15)-pentaene (74 mg, 0.180 mmol) was purified by SFC (chiral IG, 10% methanol) to afford

(-)-(7S,13R)-13-(difluoromethyl)-9-(2,6-difluorophenyl)-4,7-dimethyl-16-thia-2,3,5,8-tetrazatetracyclo [8.6.0.02,6.011, 15]hexadeca-1(10),3,5,8,11(15)-pentaene (17.8 mg, 24%) as a yellow oil. MS: 421.3 ([{³⁵Cl}M+H]⁺), ESI pos. 1H NMR (CDCl₃, 300 MHz) δ: 7.46-7.31 (m, 1H), 7.45-7.27 (m, 1H), 7.17-6.72 (m, 2H), 5.75 (br t, J=56.6 Hz, 1H), 5.73 (br t, J=57 Hz, 1H), 4.35 (q, J=6.9 Hz, 1H), 4.33 (br s, 1H), 3.20-2.97 (m, 3H), 2.49 (s, 4H), 2.45-2.44 (m, 1H), 2.07 (d, J=6.9 Hz, 4H).

(-)-(7S, 13S)-13-(difluoromethyl)-9-(2,6-difluorophenyl)-4,7-dimethyl-16-thia-2,3,5,8-tetrazatetracyclo [8.6.0.02,6.011, 15]hexadeca-1(10),3,5,8,11(15)-pentaene (14.4 mg, 19%) as a yellow oil. MS: 421.3 ([{³⁵Cl}M+H]⁺), ESI pos. 1H NMR (CDCl₃, 300 MHz) δ: 7.41-7.32 (m, 1H), 6.91 (br s, 3H), 5.86 (s, 1H), 5.68 (s, 1H), 5.69 (t, J=56.6 Hz, 1H), 5.49 (s, 1H), 4.33 (q, J=6.9 Hz, 1H), 3.20-2.94 (m, 4H), 2.56 (br dd, J=7.9, 15.7 Hz, 1H), 2.49 (s, 4H), 2.07 (d, J=6.9 Hz, 4H), 1.89-1.81 (m, 1H).

### Example 18

### (7S,13R)-13-(difluoromethyl)-9-(2,6-difluorophenyl)-N-(2-hydroxyethyl)-7-methyl-16-thia-2,3,5,8-tetrazatetracyclo [8.6.0.02,6.011,15]hexadeca-1(10),3,5,8,11(15)-pentaene-4-carboxamide

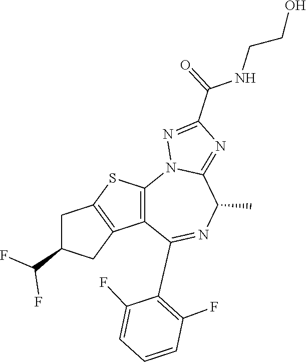 [SMILES: C[C@@H]1N=C(c2c(F)cccc2F)c2c(sc3c2C[C@@H](C(F)F)C3)-n2nc(C(=O)NCCO)nc21]

a) [2-amino-5-(difluoromethyl)-5,6-dihydro-4H-cyclopenta[b]thiophen-3-yl]-(2,6-difluorophenyl)methanone

In analogy to experiment of example 1 a, 3-(2,6-difluorophenyl)-3-oxo-propanenitrile using 3-(difluoromethyl)cyclopentanone instead of ethyl 3-oxocyclopentanecarboxylate was converted into the title compound (214 mg, 75%) which was obtained as a yellow solid, containing ca. 15% of [2-amino-6-(difluoromethyl)-5,6-dihydro-4H-cyclopenta[b]thiophen-3-yl]-(2,6-difluorophenyl)methanone. MS: 330.2 ([M+H]⁺), ESI pos.

b) tert-butyl N-[(1S)-2-[[3-(2,6-difluorobenzoyl)-5-(difluoromethyl)-5,6-dihydro-4H-cyclopenta [b]thiophen-2-yl]amino]-1-methyl-2-oxo-ethyl]carbamate

In analogy to experiment of example 5 a. [2-amino-5-(difluoromethyl)-5,6-dihydro-4H-cyclopenta [b]thiophen-3-yl]-(2,6-difluorophenyl)methanone was converted after SFC (OZ-H, 15% methanol) into the title compound (108 mg, 64%) which was obtained as a yellow solid. MS: 535.3 ([M+H]⁺), ESI pos.

c) (2S)-2-amino-N-[3-(2,6-difluorobenzoyl)-5-(difluoromethyl)-5,6-dihydro-4H-cyclopenta [b]thiophen-2-yl]propanamide

In analogy to experiment of example 2 c, tert-butyl N-[(1S)-2-[[3-(2,6-difluorobenzoyl)-5-(difluoromethyl)-5,6-dihydro-4H-cyclopenta [b]thiophen-2-yl]amino]-1-methyl-2-oxo-ethyl]carbamate was converted into the title compound (78 mg, 65%) which was obtained as a yellow solid. MS: 401.3 ([M+H]⁺), ESI pos.

d) (11S)-4-(difluoromethyl)-13-(2,6-difluorophenyl)-11-methyl-7-thia-9,12-diazatricyclo [6.5.0.02,6]trideca-1(8),2(6), 12-trien-10-one

In analogy to experiment of example 1 d. (2S)-2-amino-N-[3-(2,6-difluorobenzoyl)-5-(difluoromethyl)-5,6-dihydro-4H-cyclopenta [b]thiophen-2-yl]propanamide was converted into the title compound (221 mg, quantitative) which was obtained as a yellow solid. MS: 383.2 ([M+H]⁺). ESI pos.

e) (11S)-9-amino-4-(difluoromethyl)-13-(2,6-difluorophenyl)-11-methyl-7-thia-9,12-diazatricyclo [6.5.0.02,6]trideca-1(8),2(6),12-trien-10-one

In analogy to experiment of example 16 a. (11S)-4-(difluoromethyl)-13-(2,6-difluorophenyl)-11-methyl-7-thia-9,12-diazatricyclo [6.5.0.02,6]trideca-1(8),2(6), 12-trien-10-one was converted into the title compound (381 mg, 72%) which was obtained as a light brown solid. MS: 499.4 ([M+H]⁺). ESI pos.

f) ethyl (7S)-13-(difluoromethyl)-9-(2,6-difluorophenyl)-7-methyl-16-thia-2,3,5,8-tetrazatetracyclo [8.6.0.02,6.011.15]hexadeca-1(10),3.5.8.11(15)-pentaene-4-carboxylate

To a solution of (11S)-9-amino-4-(difluoromethyl)-13-(2,6-difluorophenyl)-11-methyl-7-thia-9,12-diazatricyclo [6.5.0.02,6]trideca-1(8),2(6), 12-trien-10-one (245 mg, 0.617 mmol) in toluene (1.3 mL) was added ethyl 2-ethoxy-2-iminoacetate (447 mg, 430 μL, 3.08 mmol). The reaction mixture was stirred at 80° C. for 2 h, then at 120° C. for additional 2 h. After this time, p-toluenesulfonic acid monohydrate (235 mg, 1.23 mmol) was added and the reaction mixture was stirred at 120° C. for 24 h. The mixture was diluted with ethyl acetate and a 1:1 mixture of water and saturated aqueous sodium bicarbonate. The aqueous phase was extracted with ethyl acetate. The organic phase was dried (Na₂SO₄) and concentrated in vacuo. The residue was purified by preparative HPLC (YMC-Triart C_{18,} water containing 0.1% formic acid/acetonitrile) to afford the title compound (140 mg, 48%) as a purple solid. MS: 479.2 ([M+H]⁺), ESI pos.

g) (7S)-13-(difluoromethyl)-9-(2,6-difluorophenyl)-7-methyl-16-thia-2,3,5,8-tetrazatetracyclo [8.6.0.02,6.011,15]hexadeca-1(10),3,5,8,11(15)-pentaene-4-carboxylic acid

To a solution of ethyl (7S)-13-(difluoromethyl)-9-(2,6-difluorophenyl)-7-methyl-16-thia-2,3,5,8-tetrazatetracyclo [8.6.0.02,6.011, 15]hexadeca-1(10),3,5,8, 11(15)-pentaene-4-carboxylate (140 mg, 0.293 mmol) and methanol (5.9 mL) was added sodium hydroxide (47 mg, 1.17 mmol). The reaction mixture was stirred at room temperature for 2 h, before being acidified by addition of HCl (1.0 M, 4 mL). The aqueous phase was extracted with dichloromethane (3×20 mL). The combined organic phases were dried (Na₂SO₄) and concentrated in vacuo to afford the title compound (134 mg, 98%) as a light brown solid. MS: 451.1 ([M+H]⁺), ESI pos.

h) (75,13R)-13-(difluoromethyl)-9-(2,6-difluorophenyl)-N-(2-hydroxyethyl)-7-methyl-16-thia-2,3,5,8-tetrazatetracyclo [8.6.0.02,6.011,15]hexadeca-1(10),3,5,8,11(15)-pentaene-4-carboxamide

To a solution of (7S)-13-(difluoromethyl)-9-(2,6-difluorophenyl)-7-methyl-16-thia-2,3,5,8-tetrazatetracyclo [8.6.0.02,6.011, 15]hexadeca-1(10),3,5,8, 11(15)-pentaene-4-carboxylic acid (67 mg, 0.144 mmol) in N,N-dimethylformamide (1.0 mL) was added (1-hydroxy-1H-benzotriazolato-o)tri-1-pyrrolidinylphosphorus hexafluorophosphate (113 mg, 0.216 mmol), 2-aminoethanol hydrochloride (42 mg, 0.433 mmol) and N,N-diisopropylethy lamine (25 μL, 0.144 mmol). The reaction mixture was stirred at room temperature for 16 h, then diluted with water (5 mL) and ethyl acetate (5 mL). The aqueous phase was extracted with ethyl acetate (3×5 mL). The organic phases were combined, washed with brine, dried (Na₂SO₄), filtered and concentrated in vacuo. The residue was purified by preparative HPLC (Gemini NX, water containing 0.1% formic acid/acetonitrile), followed by SFC (chiral IH, 20% methanol) to afford the (-)-title compound (6.5 mg, 24%) as a white solid. MS: 494.2 ([M+H]⁺), ESI pos. 1H NMR (CDCl₃, 300 MHZ) δ: 7.66 (s, 1H), 7.47-7.30 (m, 1H), 6.91 (br s, 2H), 5.75 (d, J=4.2 Hz, 1H), 6.10-5.46 (m, 1H), 4.42 (d, J=6.9 Hz, 1H), 3.86 (br s, 2H), 3.74-3.61 (m, 2H), 3.14 (br d, J=2.6 Hz, 2H), 3.03 (s, 1H), 2.62-2.43 (m, 2H), 2.12-2.04 (m, 2H), 2.16-1.91 (m, 1H).

### Example 19

### [(7S,13R)-13-(difluoromethyl)-9-(2,6-difluorophenyl)-7-methyl-16-thia-2,3,5,8-tetrazatetracyclo [8.6.0.02,6.011,15]hexadeca-1(10),3,5,8,11(15)-pentaen-4-yl]-(1,1-dioxo-1,4-thiazinan-4-yl)methanone

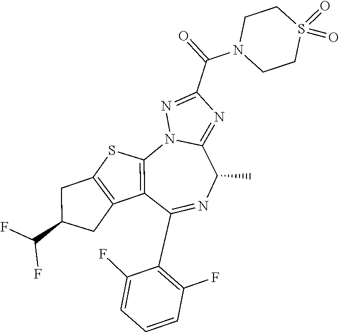 [SMILES: C[C@@H]1N=C(c2c(F)cccc2F)c2c(sc3c2C[C@@H](C(F)F)C3)-n2nc(C(=O)N3CCS(=O)(=O)CC3)nc21]

In analogy to experiment of example 18 h, (7S)-13-(difluoromethyl)-9-(2,6-difluorophenyl)-7-methyl-16-thia-2,3,5,8-tetrazatetracyclo [8.6.0.02,6.011,15]hexadeca-1(10),3,5,8,11(15)-pentaene-4-carboxylic acid was converted into the (-)-title compound (16 mg, 12%) which was obtained as a white solid. MS: 568.1 ([M+H]⁺), ESI pos. 1H NMR (CDCl₃, 300 MHZ) δ: 7.47-7.33 (m, 1H), 7.18-6.76 (m, 2H), 5.75 (br t, J=56.4 Hz, 1H), 5.30 (s, 1H), 4.48-4.23 (m, 5H), 3.28-3.11 (m, 6H), 3.11-2.97 (m, 1H), 2.50 (td, J=2.5, 15.8 Hz, 1H), 2.09 (d, J=6.9 Hz, 3H).

### Example 20

### (7S,13R)-13-(difluoromethyl)-9-(2,6-difluorophenyl)-3,7-dimethyl-16-thia-2,5,8-triazatetracyclo [8.6.0.02,6.011,15]hexadeca-1(10),3,5,8,11(15)-pentaene

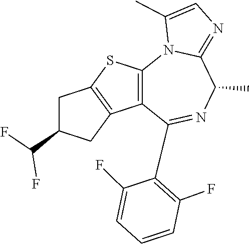 [SMILES: Cc1cnc2n1-c1sc3c(c1C(c1c(F)cccc1F)=N[C@H]2C)C[C@@H](C(F)F)C3]

a) 4-(difluoromethyl)-13-(2,6-difluorophenyl)-11-methyl-7-thia-9,12-diazatricyclo [6.5.0.02,6]trideca-1(8),2(6), 12-triene-10-thione

In analogy to experiment of example 1 e, (11S)-4-(difluoromethyl)-13-(2,6-difluorophenyl)-11-methyl-7-thia-9,12-diazatricyclo [6.5.0.02,6]trideca-1(8),2(6), 12-trien-10-one was converted into the title compound (687 mg, 73%) which was obtained as an orange solid. MS: 399.3 ([M+H]⁺), ESI pos.

b) 4-(difluoromethyl)-13-(2,6-difluorophenyl)-11-methyl-7-thia-9,12-diazatricyclo [6.5.0.02,6]trideca-1(8),2(6), 12-trien-10-imine

A mixture of 4-(difluoromethyl)-13-(2,6-difluorophenyl)-11-methyl-7-thia-9,12-diazatricyclo [6.5.0.02,6]trideca-1(8),2(6), 12-triene-10-thione (687 mg, 1.72 mmol) in ammonia (7.0 M in methanol, 7.4 mL, 51.7 mmol) was stirred at 60° C. for 16 h in a sealed tube. The reaction mixture was concentrated in vacuo. The residue was purified by flash column chromatography (silica, 0% to 5% methanol in dichloromethane) to afford the title compound (568 mg, 86%) as a yellow solid. MS: 382.2 ([M+H]⁺), ESI pos.

c) (7S,13R)-13-(difluoromethyl)-9-(2,6-difluorophenyl)-3,7-dimethyl-16-thia-2,5,8-triazatetracyclo [8.6.0.02,6.011,15]hexadeca-1(10),3,5,8,11(15)-pentaene

A mixture of 4-(difluoromethyl)-13-(2,6-difluorophenyl)-11-methyl-7-thia-9,12-diazatricyclo [6.5.0.02,6]trideca-1(8),2(6), 12-trien-10-imine (592 mg, 1.49 mmol), propargy lamine (763 μL, 11.91 mmol) and p-toluenesulfonic acid monohydrate (57 mg, 0.298 mmol) was stirred at 120° C, for 4 h in a sealed tube. The reaction mixture was concentrated in vacuo, then purified by flash column chromatography (silica, 1% methanol in dichloromethane), followed by SFC (chiral OZ-H, 10% methanol) to afford the (-)-title compound (18 mg, 13%) as a white solid. MS: 420.1 ([M+H]⁺), ESI pos. 1H NMR (CDCl₃, 300 MHz) δ: 7.40-7.29 (m, 1H), 7.08-6.79 (m, 3H), 5.77 (br t, J=57 Hz, 1H), 5.30 (s, 1H), 4.22 (q, J=6.2 Hz, 1H), 3.22-2.96 (m, 3H), 2.53 (br d, J=15.3 Hz, 1H), 2.43 (s, 3H), 2.06 (br d, J=6.6 Hz, 5H).

### Example 21 and Example 22

### [(7S,13R)-13-(difluoromethyl)-9-(2,6-difluorophenyl)-7-methyl-16-thia-2,5,8-triazatetracyclo [8.6.0.02,6.011,15]hexadeca-1(10),3,5,8,11(15)-pentaen-3-yl]methanol

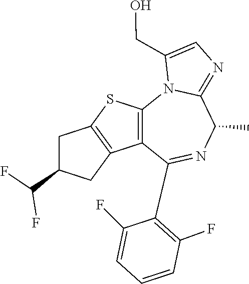 [SMILES: C[C@@H]1N=C(c2c(F)cccc2F)c2c(sc3c2C[C@@H](C(F)F)C3)-n2c(CO)cnc21]

[(7S,13S)-13-(difluoromethyl)-9-(2,6-difluorophenyl)-7-methyl-16-thia-2,5,8-triazatetracyclo [8.6.0.02,6.011,15]hexadeca-1(10),3,5,8,11(15)-pentaen-3-yl]methanol

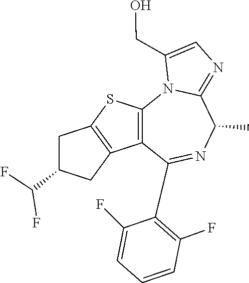 [SMILES: C[C@@H]1N=C(c2c(F)cccc2F)c2c(sc3c2C[C@H](C(F)F)C3)-n2c(CO)cnc21]

In a flame dried flask 13-(difluoromethyl)-9-(2,6-difluorophenyl)-3,7-dimethyl-16-thia-2,5,8-triazatetracyclo [8.6.0.02,6.011,15]hexadeca-1(10),3,5,8,11(15)-pentaene (200 mg, 0.415 mmol) was dissolved in 1,4-dioxane (4.15 mL) under argon atmosphere. Selenium dioxide (46 mg, 0.415 mmol) was added in one portion and the reaction was stirred at 110° C. for 5 h. The reaction mixture was allowed to cooled down then filtered over dicalite. The filtrate was concentrated in vacuo and the residue was purified by flash column chromatography (silica, 50% ethyl acetate in dichloromethane), followed by SFC (chiral IC, 15% methanol) to afford

(-)-[(7S,13R)-13-(difluoromethyl)-9-(2,6-difluorophenyl)-7-methyl-16-thia-2,5,8-triazatetracyclo [8.6.0.02,6.011,15]hexadeca-1(10),3,5,8,11(15)-pentaen-3-yl]methanol (20.7 mg, 11%) as a white solid. MS: 436.1 ([M+H]⁺), ESI pos. 1H NMR (CDCl₃, 300 MHz) δ: 7.41-7.28 (m, 1H), 7.03 (s, 3H), 5.75 (br t, J=57 Hz, 1H), 5.77 (br s, 1H), 5.30 (s, 1H), 4.94-4.61 (m, 2H), 4.27 (q, J=6.9 Hz, 1H), 3.22-2.94 (m, 2H), 2.66 (br s, 1H), 2.61-2.47 (m, 1H), 3.52-2.27 (m, 1H), 2.07 (d, J=6.9 Hz, 3H).

(-)-[(7S,13S)-13-(difluoromethyl)-9-(2,6-difluorophenyl)-7-methyl-16-thia-2,5,8-triazatetracyclo [8.6.0.02,6.011,15]hexadeca-1(10),3,5,8,11(15)-pentaen-3-yl]methanol (26.6 mg, %) as a white solid. MS: 436.1 ([M+H]⁺), ESI pos. 1H NMR (CDCl₃, 300 MHz) δ: 7.41-7.30 15 (m, 1H), 7.04 (s, 1H), 7.00-6.76 (m, 2H), 5.71 (br t, J=57 Hz, 1H), 5.30 (s, 4H), 4.96-4.58 (m, 2H), 4.24 (br d, J=6.9 Hz, 1H), 3.47 (s, 1H), 3.19-2.94 (m, 2H), 3.34-2.84 (m, 1H), 2.74-2.50 (m, 2H), 2.07 (d, J=6.9 Hz, 3H), 1.91 (br dd, J=5.9, 16.0 Hz, 1H).

### Reference Compound RE-C

(7S)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene

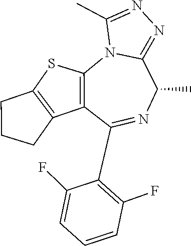 [SMILES: Cc1nnc2n1-c1sc3c(c1C(c1c(F)cccc1F)=N[C@H]2C)CCC3]

a) (2-amino-5,6-dihydro-4H-cyclopenta[b]thiophen-3-yl)-(2,6-difluorophenyl)methanone

In analogy to experiment of example 1 a, cyclopentanone was converted into the title compound (744 mg, 60%) which was obtained as a yellow foam. MS: 280.1 ([M+H]⁺), ESI pos.

b) tert-butyl N-[(1S)-2-[[3-(2,6-difluorobenzoyl)-5,6-dihydro-4H-cyclopenta[b]thiophen-2-yl]amino]-1-methyl-2-oxo-ethyl]carbamate

In analogy to experiment of example 5 a, (2-amino-5,6-dihydro-4H-cyclopenta[b]thiophen-3-yl)-(2,6-difluorophenyl)methanone was converted into the title compound (955 mg, 92%) which was obtained as a yellow solid. MS: 464.4 ([M+H]⁺), ESI pos.

c) (2S)-2-amino-N-[3-(2,6-difluorobenzoyl)-5,6-dihydro-4H-cyclopenta[b]thiophen-2-yl]propanamide

In analogy to experiment of example 15 c, tert-butyl N-[(1S)-2-[[3-(2,6-difluorobenzoyl)-5,6-dihydro-4H-cyclopenta [b]thiophen-2-yl]amino]-1-methyl-2-oxo-ethyl]carbamatewas converted into the title compound (615 mg, 83%) which was obtained as a brown solid. MS: 349.2 ([M+H]⁺), ESI pos.

d) (11S)-13-(2,6-difluorophenyl)-11-methyl-7-thia-9,12-diazatricyclo[6.5.0.02,6]trideca-1(8), 2(6), 12-trien-10-one

In analogy to experiment of example 1 d, (2S)-2-amino-N-[3-(2,6-difluorobenzoyl)-5,6-dihydro-4H-cyclopenta [b]thiophen-2-yl]propanamide was converted into the title compound (276 mg, 43%) which was obtained as a yellow foam. MS: 333.2 ([M+H]⁺), ESI pos.

e) (11S)-13-(2,6-difluorophenyl)-11-methyl-7-thia-9,12-diazatricyclo[6.5.0.02,6]trideca-1(8), 2(6), 12-triene-10-thione

In analogy to experiment of example 1 e, (11S)-13-(2,6-difluorophenyl)-11-methyl-7-thia-9,12-diazatricyclo [6.5.0.02,6]trideca-1(8),2(6), 12-trien-10-one was converted into the title compound (301 mg, quantitative) which was obtained as a brown solid. MS: 349.1 ([M+H]⁺), ESI pos.

f) (7S)-9-(2,6-difluorophenyl)-3,7-dimethyl-16-thia-2,4,5,8-tetrazatetracyclo [8.6.0.02,6.011,15]hexadeca-1(10),3,5,8,11(15)-pentaene

In analogy to experiment of example 1 f, (11S)-13-(2,6-difluorophenyl)-11-methyl-7-thia-9,12-diazatricyclo [6.5.0.02,6]trideca-1(8),2(6), 12-triene-10-thione was converted into the (-)-title compound (21.5 mg, 25%) which was obtained as a light brown solid. MS: 371.3 ([M+H]⁺), ESI pos. 1H NMR (CDCl₃, 300 MHz) δ: 7.35 (tt, J=6.3, 8.5 Hz, 1H), 7.27 (s, 1H), 6.92 (br s, 2H), 5.30 (s, 1H), 4.39 (br d, J=5.8 Hz, 1H), 3.48 (s, 1H), 2.96-2.88 (m, 2H), 2.70 (s, 3H), 2.46-2.20 (m, 3H), 2.11 (d, J=6.9 Hz, 3H), 1.90 (br d, J=14.7 Hz, 1H), 1.87 (br d, J=14.5 Hz, 1H).

### Assay Procedures

Membrane Preparation and Binding Assay for γ1-containing GABA_{A} Subtypes

The affinity of compounds at GABA_{A} γ1 subunit-containing receptors was measured by competition for [³H]RO7239181 (67.3 Ci/mmol: Roche) binding to membranes from HEK293F cells (ThermoFisher R79007) expressing human (transiently transfected) receptors of composition α5β2γ1, α2β2γ1, α1β2γ1. For better protein expression of the α2 subunit-containing receptors, the 28 amino acid long signal peptide (Metl to Ala28) of the human GABA_{A} α2 subunit was substituted by the 31 amino acid long signal peptide (Met1 to Ser31) of human GABA_{A} α5 subunit.

Harvested pellets from HEK293F cells expressing the different GABA_{A} receptor subtypes were resuspended in Mannitol Buffer pH 7.2-7.4 (Mannitol 0.29 M, Tricthylamine 10 mM. Acetic acid 10 mM. EDTA 1 mM plus protease inhibitors (20 tablets Complete. Roche Diagnostics Cat. No. 05 056 489 001 per liter)), washed two times and then resuspended at 1:10 to 1:15 dilution in the same buffer. Cell disruption was performed by stirring the suspension in a Parr vessel #4637 at 435 psi for 15 minutes, and then the suspensions were centrifuged at 1000×g for 15 minutes at 4° C. (Beckman Avanti J-HC: rotor JS-4.2). The supernatant (S1) was transferred in a 21 Schott flask and the pellet (P1) was resuspended with Mannitol Buffer up to 175 ml. The resuspended pellet was transferred into a 250 ml Corning centrifugal beaker and centrifuged at 1500 ×g for 10 minutes at 4° C. (Beckman Avanti J-HC: rotor JS-4.2). The supernatant (S1) was then transferred in the 21 Schott flask and the pellet was discarded. The supernatants (S1) were centrifuged in 500ml Beckman polypropylene centrifugal beaker at 15 000×g for 30 minutes at 4° C. (Beckman Avanti J-20 XP; rotor JLA-10.500). The pellet (P2) was resuspended with Mannitol Buffer 1:1 and frozen at −80° C. The supernatant (S2) was centrifuged in 100 ml Beckman polypropylene centrifugal tubes at 48000×g for 50 minutes at 4ºC (Beckman Avanti J-20 XP: rotor JA-18). The supernatant (S3) was discarded and the pellet (P3) was resuspended with 1:1 Mannitol Buffer. The P2 and P3 protein concentration was determined with the BIORAD Standard assay method with bovine serum albumin as standard and measured on the NANO-Drop 1000. The membrane suspension was aliquots (500μl per tube) and stored at −80° C. until required.

Membrane homogenates were resuspended and polytronised (Polytron PT1200E Kinematica AG) in Potassium Phosphate 10 mM, KCl 100 mM binding buffer at pH 7.4 to a final assay concentration determined with a previous experiment.

Radioligand binding assays were carried out in a volume of 200 μL (96-well plates) which contained 100 μL of cell membranes. [³H]RO7239181 at a concentration of 1.5 nM (α5β2γ1) or 20-30 nM (α1β2γ1, α2β2γ1) and the test compound in the range of [0.3-10000]33 10⁻⁹ M. Nonspecific binding was defined by 10×10⁶ (α5β2γ1) and 30×10⁶ M RO7239181 and typically represented less than 5% (α5β2γ1) and less than 20% (α1β2γ1, α2β2γ1) of the total binding. Assays were incubated to equilibrium for 1 hour at 4° C. and then, membranes were filtered onto unifilter (96-well white microplate with bonded GF/C filters preincubated 20-50 minutes in 0.3% Polyethylenimine) with a Filtermate 196 harvester (Packard BioScience) and washed 4 times with cold Potassium Phosphate 10 mM pH 7.4. KCl 100 mM binding buffer. After anhydrousing, filter-retained radioactivity was detected by liquid scintillation counting. Kᵢ values were calculated using Excel-Fit (Microsoft) and are the means of two determinations.

The compounds of the accompanying examples were tested in the above described assays, and the preferred compounds were found to possess a Kᵢ value for the displacement of [³H]RO7239181 from GABA_{A} Yl subunit-containing receptors (e.g, α5β2γ1, α2β2γ1, α1β2γ1) of 100 nM or less. Most preferred are compounds with a Ki (nM)<50. Representative test results, obtained by the above described assay measuring binding affinity to HEK293 cells expressing human (h) receptors, are shown in the Table 1.

Preparation of [³H]RO7239181, 6-chloro-5-(2,6-difluorophenyl)-7-methyl-1-(tritritiomethyl)-3H-1,4-benzodiazepin-2-one

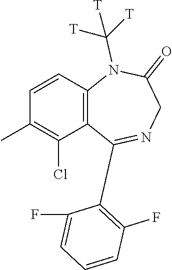

a) 5-chloro-2-methyl-3,1-benzoxazin-4-one

A solution of 2-amino-6-chlorobenzoic acid (250 g, 1.46 mol) in acetic anhydride (1250 mL) was stirred at 140° C, for 2 h. The reaction mixture was concentrated in vacuo. The resulting crude residue was suspended in ethyl acetate (1000 mL), stirred for 30 min, filtered and dried in vacuo to afford the title compound (238 g, 84%) as a grey solid. 1H NMR (DMSO-d6, 400 MHZ): δ: 7.80 (app t, J=8.0 Hz, 1H), 7.62 (d, J=8.0 Hz, 1H), 7.49 (d, J=7.6 Hz, 1H), 2.36 (s, 3H).

b) N-[3-chloro-2-(2,6-difluorobenzoyl)phenyl]acetamide

To a solution of 5-chloro-2-methyl-3,1-benzoxazin-4-one (100 g, 511.2 mmol) and 2-bromo-1,3-difluorobenzene (118.4 g, 613.5 mmol) in tetrahydrofuran (1000 mL) was added dropwise i-PrMgCl·LiCl (1.3 M, 500 mL, 650 mmol) at −70° C. under nitrogen. The mixture was allowed to warm up to room temperature within 1 h, quenched with saturated aqueous ammonium chloride (1500 mL) and extracted with ethyl acetate (2×1500 mL). The organic phase was washed with brine (2000 mL), dried (Na₂SO₄) and concentrated in vacuo. The residue was suspended in ethyl acetate (150 mL). The resulting suspension was stirred at room temperature for 20 min, filtered and dried in vacuo to afford the title compound (113 g, 71%) as an off-white solid. 1H NMR (DMSO-d6, 400 MHZ): δ: 9.85 (s, 1H), 7.65-7.45 (m, 1H), 7.40 (t, J=7.2 Hz, 1H), 7.38-7.34 (m, 2H), 7.16 (t, J=8.8 Hz, 2H), 1.85 (s, 3H).

c) (2-amino-6-chloro-phenyl)-(2,6-difluorophenyl)methanone

To a solution of N-[3-chloro-2-(2,6-difluorobenzoyl)phenyl]acetamide (113 g, 364.9 mmol) in ethanol (250 mL) was added aqueous hydrochloric acid (12 M, 200 mL). The reaction mixture was stirred at 100° C. for 1 h, then diluted with ethyl acetate (1100 mL). The organic phase was washed with water (1100 mL), saturated aqueous sodium bicarbonate (1100 mL) and brine (1100 mL).

dried over sodium sulfate and concentrated in vacuo. Petroleum ether (120 mL) was added to the crude and the suspension was stirred at room temperature for 20 min. The solid was filtered and dried to afford the title compound (88 g, 90%) as a yellow solid. 1H NMR (DMSO-d6, 400 MHZ): δ: 7.62-7.56 (m, 1H), 7.21-7.15 (m, 3H), 6.83 (d. J=7.6 Hz, 1H), 6.74 (s, 2H), 6.58 (d. J=7.6 Hz. 1H).

d) (6-amino-3-bromo-2-chloro-phenyl)-(2,6-difluorophenyl)methanone

To a solution of (2-amino-6-chloro-phenyl)-(2,6-difluorophenyl)methanone (88.0 g, 328.8 mmol) in dichloromethane (225 mL) and N.N-dimethylformamide (225 mL) was added 1-bromopyrrolidine-2,5-dione (64.4 g, 362 mmol) at 0° C. The reaction mixture was stirred at 30° C. for 1 h. The mixture was diluted with dichloromethane (600 mL) and washed with water (500 mL) and brine (4×500 mL), dried (Na₂SO₄) and concentrated in vacuo. The residue was purified by chromatography (silica, petroleum ether/ethyl acetate, 1:0 to 2:1). The solid was suspended in petroleum ether (200 mL) and stirred at room temperature for 20 min. The suspension was filtered and the solid was dried in vacuo to afford the title compound (96.0 g, 84%) as a yellow solid. MS: 345.9 ([{⁷⁹Br, ³⁵Cl}M+H]⁺), 347.8 ([{⁸¹Br, ³⁵Cl or “”Br, ³⁷Cl}M+H]⁺). ESI pos.

e) 7-bromo-6-chloro-5-(2,6-difluorophenyl)-1.3-dihydro-1.4-benzodiazepin-2-one

To a solution of (6-amino-3-bromo-2-chloro-phenyl)-(2,6-difluorophenyl)methanone (25.0 g, 72.1 mmol) in pyridine (625 mL) was added ethyl 2-aminoacetate hydrochloride (70.5 g, 505 mmol). The reaction mixture was stirred at 135° C. for 36 h. The reaction mixture was concentrated in vacuo to remove pyridine. The residue was diluted with ethyl acetate (2000 mL) and washed with aqueous HCl (1.0 M, 3×1500 mL), water (2000 mL) and brine (2 × 1000 mL), dried (Na₂SO₄). filtered and concentrated in vacuo. The crude product was purified by flash column chromatography (silica, petroleum ether/ethyl acetate 10:1 to 2:1) to afford the title compound (10.1 g, 12%) as an off-white solid. MS: 385.0 ([{⁷⁹Br, ³⁵Cl}M+H]⁺). ESI pos.

f) 6-chloro-5-(2,6-difluorophenyl)-7-methyl-1,3-dihydro-1,4-benzodiazepin-2-one

A microwave tube was charged with 7-bromo-6-chloro-5-(2,6-difluorophenyl)-1.3-dihydro-1.4-benzodiazepin-2-one (450 mg, 1.17 mmol), trimethylboroxine (205 mg, 228 μL, 1.63 mmol). potassium carbonate (242 mg, 1.75 mmol) and tetrakis(triphenylphosphine)palladium (0) (67.4 mg. 58.4 umol). Degassed 1.4-dioxane (8.1 mL) and H₂O (2.7 ml) were added then the vial was capped. The suspension was reacted in microwave at 130° C. for 30 min to give complete conversion. The mixture was evaporated, treated with sat, aq. NaHCO₃ (20 mL) and extracted with EtOAc (2×20 mL). The organic layers were dried (Na₂SO₄), filtered and solvents were evaporated. The residue was purified by flash column chromatography (silica, 40 g, CH₂Cl₂/EtOAc in heptane 10% to 40% to 70%) to give the title compound (344 mg, 92%) as light yellow solid. MS (ESI): 321.1 ([M+H]⁺).

g) 6-chloro-5-(2,6-difluorophenyl)-7-methyl-1-(tritritiomethyl)-3H-1,4-benzodiazepin-2-one

To a solution of [³H]methyl nosylate (1.85 GBq, 50 mCi, 0.61 umol) in THF (200 μL) were added the N-desmethyl precursor 6-chloro-5-(2,6-difluorophenyl)-7-methyl-1,3-dihydro-1,4-benzodiazepin-2-one (0.43 mg, 1.34 umol) dissolved in THF (200 μL) and 10 equivalents of sodium tert-buty late (0.5 M in THF, 13.4 umol). After stirring for 4 h at room temperature the reaction mixture was treated with H2O, evaporated, and the crude product was purified by HPLC (X-Terra Prep RP-18, 10×150 mm, MeCN/H2O (containing 5% of MeCN) 40:60, 4 ml/min, 230 nm). The pure tritium-labeled compound was isolated by solid phase extraction (Sep-Pak Plus

C₁₈) and eluted from the cartridge as ethanolic solution to yield 1.6 GBq (43.2 mCi) of the target compound in >99% radio-chemical purity and a specific activity of 2.49 TBq/mmol (67.3 Ci/mmol) as determined by mass spectrometry (MS). The identity of the labeled compound was confirmed by HPLC (by co-injecting the unlabeled reference standard) and by MS.

MS: m/z=335 [M(H)+H]⁺ (16%), 337 [M(³H)+H]⁺ (0%), 339 [M(³H₂)+H]⁺ (16%), 341 [M(³H3)+H]⁺ (68%).

Membrane Preparation and Binding Assay for γ2-containing GABA_{A} Subtypes

The affinity of compounds at GABA_{A} γ2 subunit-containing receptors was measured by competition for [³H]Flumazenil (81.1 Ci/mmol; Roche) binding to HEK293F cells expressing human (transiently transfected) receptors of composition α1β3γ2.

Harvested pellets from HEK293F cells expressing the different GABA_{A} γ2 receptor subtypes were resuspended in Mannitol Buffer pH 7,2-7,4 and processed as described above for the cells expressing the GABA_{A} γ1 subunit-containing receptors.

Radioligand binding assays were carried out in a volume of 200 μL (96-well plates) which contained 100 μL of cell membranes, [³H]Flumazenil at a concentration of 1 nM and the test compound in the range of [0.1·10⁻³−10]×10⁻⁶ M. Nonspecific binding was defined by 105 M Diazepam and typically represented less than 5% of the total binding. Assays were incubated to equilibrium for 1 hour at 4° C, and harvested onto GF/C uni-filters (Packard) by filtration using a Packard harvester and washing with ice-cold wash buffer (50 mM Tris; pH 7.5). After anhydrousing, filter-retained radioactivity was detected by liquid scintillation counting. Kᵢ values were calculated using Excel-Fit (Microsoft) and are the means of two determinations.

The compounds of the accompanying examples were tested in the above described assay, and the preferred compounds were found to possess large Kᵢ value for displacement of [³H]Flumazenil from the α1β3γ2 subtype of the human GABA_{A} receptor of 100 nM or above. Most preferred are compounds with a Kᵢ α1β3γ2 (nM)>300. In a preferred embodiment the compounds of the invention are binding selectively for the γ1 subunit-containing GABA_{A} receptors relative to γ2 subunit-containing GABA_{A} receptors. In particular, compounds of the present invention have γ2/γ1 selectivity ratio defined as “Kᵢ α1β3γ2 (nM)/K; α2β2γ1 (nM)” above 10-fold, or LogSel defined as “Log[Kᵢ α1β3γ2 (nM)/Kᵢ α2β2γ1 (nM)]” above 1. Representative test results, obtained by the above described assay measuring binding affinity to HEK293 cells expressing human (h) receptors, are shown in the Table 1 below.

TABLE 1

| | Ki | Ki | Ki | Ki | γ2/γ1 | |
|---|---|---|---|---|---|---|
| Ex- | h-GABA_{A} | h-GABA_{A} | h-GABA_{A} | h-GABA_{A} | Selec- | |
| am- | α5β2γ1 | α2β2γ1 | α1β2γ1 | α1β3γ2 | tivity | |
| ple | (nM) | (nM) | (nM) | (nM) | Ratio | LogSel |
| 1 | 8.8 | 25.4 | 47.9 | 1246.6 | 49.0 | 1.69 |
| 2 | 16.3 | 42.7 | ND | 2162.8 | 50.7 | 1.71 |
| 3 | 1.8 | 2.9 | 16.8 | 256.7 | 88.4 | 1.95 |
| 4 | 1.2 | 4.6 | ND | 343.1 | 74.8 | 1.87 |
| 5 | 1.2 | 3.4 | 14.4 | 227.8 | 66.5 | 1.82 |
| 6 | 3.4 | 6.6 | ND | 647.2 | 97.7 | 1.99 |
| 7 | 2.1 | 6.4 | ND | 304.3 | 47.3 | 1.67 |
| 8 | 1.9 | 4.6 | ND | 455.4 | 99.2 | 2.00 |
| 9 | 1.6 | 3.6 | ND | 365.7 | 101.3 | 2.01 |
| 10 | 3.0 | 3.0 | ND | 3224.7 | 1090.8 | 3.04 |
| 11 | 3.4 | 10.3 | ND | 1124.1 | 108.6 | 2.04 |
| 12 | 3.7 | 13.4 | ND | 2359.1 | 176.0 | 2.25 |
| 13 | 5.9 | 16.2 | ND | 705.0 | 43.5 | 1.64 |
| 14 | 1.6 | 2.8 | ND | 100.9 | 36.2 | 1.56 |
| 15 | 1.4 | 6.3 | ND | 101.1 | 16.1 | 1.21 |
| 16 | 1.0 | 2.3 | ND | 448.1 | 191.1 | 2.28 |
| 17 | 31.6 | 57.9 | ND | 6642.6 | 114.8 | 2.06 |
| 18 | 2.5 | 5.7 | ND | 614.1 | 107.2 | 2.03 |
| 19 | 4.6 | 5.4 | ND | 873.4 | 162.3 | 2.21 |
| 20 | 3.6 | 2.24 | ND | 190.5 | 53.4 | 1.73 |
| 21 | 4.7 | 2.97 | ND | 605.7 | 127.6 | 2.11 |
| 22 | 44.3 | 10.16 | ND | 2512.6 | 56.8 | 1.75 |

### Functional Expression of GABA_{A} Receptors:

### Xenopus Oocytes Preparation

Xenopus laevis oocytes at maturation stages V-VI were used for the expression of cloned mRNA encoding GABA_{A} receptor subunits. Oocytes ready for RNA micro-injection were bought from Ecocyte, Castrop-Rauxel, Germany and stored in modified Barth's medium (composition in mM: NaCl 88, KCl 1, NaHCO₃ 2.4, HEPES 10, MgSO₄ 0.82, CaNO₃ 0.33, CaCl₂ 0.33, pH=7.5) at 20 C. until the experiment.

### Xenopus Oocytes Microinjection

Oocytes were plated in 96-well plates for microinjection using the Roboinject automated instrument (MultiChannelSystems, Reutlingen, Germany). Approximately 50 nL of an aqueous solution containing the RNA transcripts for the subunits of the desired GABA_{A} receptor subtype was injected into each oocyte. RNA concentrations ranged between 20 and 200 pg/μL/subunit and were adjusted in pilot experiments to obtain GABA responses of a suitable size and a maximal effect of Flunitrazepam, Triazolam and Midazolam, reference benzodiazepine positive allosteric modulators (PAM) at the GABA_{A} receptor benzodiazepine (BZD) binding site. Oocytes were kept in modified Barth's medium (composition in mM: NaCl 88, KCl 1, NaHCO₃ 4, HEPES 10, MgSO_{4 0.82,} CaNO3 0.33, CaCl2 0.33, pH=7.5) at 20° C. until the experiment.

### Electrophysiology

Electrophysiological experiments were performed using the Roboocyte instrument (MultiChannelSystems, Reutlingen, Germany) on days 3 to 5 after the micro-injection of mRNA. During the experiment the oocytes were constantly superfused by a solution containing (in mM) NaCl 90, KCl 1, HEPES 5, MgCl: 1, CaCl, 1 (pH 7.4). Oocytes were impaled by two glass microelectrodes (resistance: 0.5-0.8 MS2) which were filled with a solution containing KCl 1M +K-acetate 1.5 M and voltage-clamped to −80 mV. The recordings were performed at room temperature using the Roboocyte two-electrode voltage clamp system (Multichannelsystem). After an initial equilibration period of 1.5 min GABA was added for 1.5 min at a concentration evoking approximately 20% of a maximal current response (EC₂₀). After another rest interval of 2.5 min GABA was again added evoking a response of similar amplitude and shape. 0.5 min after the onset of this second GABA application the test compound, at a concentration corresponding to approximatively 30-fold its Kᵢ α2β2γ1, was added while GABA was still present. Current traces were recorded at a digitization rate of 10 Hz during and shortly before and after the GABA application.

Each compound and concentration was tested on at least 3 oocytes. Different oocytes were used for different compound concentrations. The reference PAMs, Flunitrazepam, Triazolam and Midazolam, potentiated the GABA-induced current in α2β2γ1 GABA_{A} receptor subtype expressing oocytes by approximatively 60%.

### Data Analysis

For the analysis, the digitized current traces of the first and second GABA response were superimposed and, if necessary, rescaled to equal maximal amplitudes. The ratio between the two responses during the time interval of test compound application was calculated point by point. The extremum of the resulting “ratio trace” was taken as the efficacy (“Fold increase”) of the compound expressed as “% modulation of GABA EC” (100* (Fold increase-1)).

The results are shown in Table 2.

TABLE 2

| | Ki | Fold increase | |
|---|---|---|---|
| | h-GABA_{A} | h-GABA-A | |
| | α2β2γ1 | α2β2γ1 | Efficacy |
| Example | (nM) | oocyte @ 30-fold Ki | (GABA)% |
| 1 | 25.4 | 1.54 | 54 |
| 2 | 42.7 | 1.35 | 35 |
| 3 | 2.9 | 1.54 | 54 |
| 4 | 4.6 | 1.51 | 51 |
| 5 | 3.4 | 1.41 | 41 |
| 6 | 6.6 | 1.85 | 85 |
| 7 | 6.4 | 1.71 | 71 |
| 8 | 4.6 | 1.37 | 37 |
| 9 | 3.6 | 1.38 | 38 |
| 10 | 3.0 | 1.47 | 47 |
| 11 | 10.3 | 1.44 | 44 |
| 12 | 13.4 | 1.62 | 62 |
| 13 | 16.2 | 1.51 | 51 |
| 14 | 2.8 | 1.68 | 68 |
| 15 | 6.3 | 1.45 | 45 |
| 16 | 2.3 | 1.32 | 32 |
| 17 | 57.9 | 1.25 | 25 |
| 18 | 5.7 | 1.65 | 65 |
| 19 | 5.4 | 1.43 | 43 |
| 20 | 2.24 | 1.70 | 70 |
| 21 | 2.97 | 1.68 | 68 |
| 22 | 10.16 | — | — |

### Reference Compounds

Benzodiazepines reference compounds (classical marketed benzodiazepines) and reference thieno-diazepines listed below were tested for their affinity towards the GABA_{A} receptor α1 β2γ1 and α2β2γ1 subtypes as well as in the GABA receptor α1β3γ2 subtype. The results are shown in Table 3.

TABLE 3

| | Ki | Ki | Ki | | |
|---|---|---|---|---|---|
| | h-GABA_{A} | h-GABA_{A} | h-GABA_{A} | γ2/γ1 | |
| | α1β2γ1 | α2β2γ1 | α1β3γ2 | Selectivity | |
| Example | (nM) | (nM) | (nM) | Ratio | LogSel |
| Alprazolam | 5923 | 3945 | 19.6 | 0.0050 | −2.3 |
| Triazolam | 44.2 | 46.2 | 1.5 | 0.032 | −1.5 |
| Midazolam | 1153.2 | 737.7 | 5.0 | 0.0068 | −2.2 |
| RE-A | ND | 17.5 | 6.6 | 0.38 | −0.42 |
| RE-B | ND | 734.1 | 1221.6 | 1.66 | 0.22 |
| RE-C | 2.59 | 20.4 | 17.2 | 0.84 | −0.07 |

Alprazolam 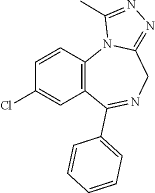 [SMILES: Cc1nnc2n1-c1ccc(Cl)cc1C(c1ccccc1)=NC2]
Triazolam 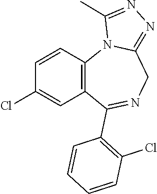 [SMILES: Cc1nnc2n1-c1ccc(Cl)cc1C(c1ccccc1Cl)=NC2]
Midazolam 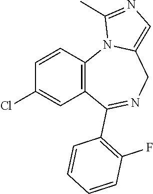 [SMILES: Cc1ncc2n1-c1ccc(Cl)cc1C(c1ccccc1F)=NC2]
RE-A 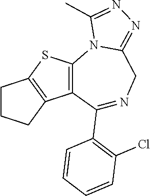 [SMILES: Cc1nnc2n1-c1sc3c(c1C(c1ccccc1Cl)=NC2)CCC3]
RE-B 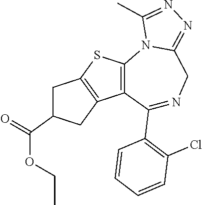 [SMILES: CCOC(=O)C1Cc2sc3c(c2C1)C(c1ccccc1Cl)=NCc1nnc(C)n1-3]
RE-C

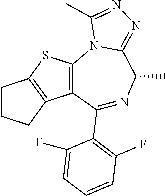 [SMILES: Cc1nnc2n1-c1sc3c(c1C(c1c(F)cccc1F)=N[C@H]2C)CCC3]

WO 94/04537 discloses reference compound RE-A and DE 3724031 discloses reference compound RE-B. Reference example RE-C has been prepared as described herein.

### Preparation of Pharmaceutical Compositions Comprising Compounds of the Invention

Tablets comprising compounds of formula (1) are manufactured as follows:

mg/tablet

| Ingredient | 5 | 25 | 100 | 500 |
|---|---|---|---|---|
| Compound of formula I | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.

2. Dry the granules at 50° C.

3. Pass the granules through suitable milling equipment.

4. Add ingredient 5 and mix for three minutes; compress on a suitable press.
Capsules comprising compounds of formula (1) are manufactured as follows:

mg/capsule

| Ingredient | 5 | 25 | 100 | 500 |
|---|---|---|---|---|
| Compound of formula I | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.

2. Add ingredients 4 and 5 and mix for 3 minutes.

3. Fill into a suitable capsule.
A compound of formula I lactose and corn starch are firstly mixed in a mixer and then in a comminuting machine. The mixture is returned to the mixer; the talc is added thereto and mixed thoapproximatively. The mixture is filled by machine into suitable capsules, e.g, hard gelatin capsules.
Injection solutions comprising compounds of formula (1) are manufactured as follows:

| Ingredient | mg/injection solution. |
|---|---|
| Compound of formula I | 3 |
| Polyethylene Glycol 400 | 150 |
| acetic acid | q.s. ad pH 5.0 |
| water for injection solutions | ad 1.0 ml |

1. A compound of formula (I) 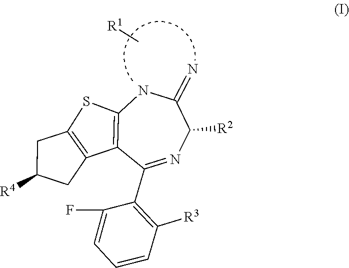
or a pharmaceutically acceptable salt thereof, wherein: 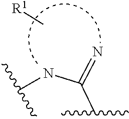
is selected from: 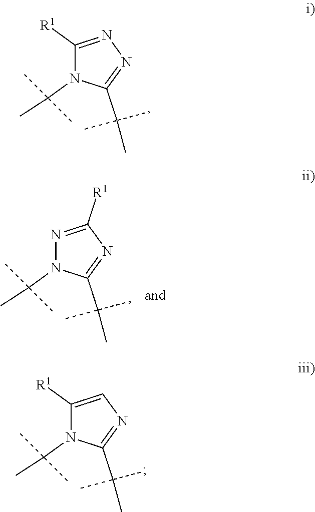
R¹ is selected from hydrogen, C₁-C₆-alkyl, C₁-C₆-alkoxy, C₁-C₆-alkoxy-C₁-C₆-alkyl, hydroxy-C₁-C₆-alkyl, C₃-C₁₀-cycloalkyl, amino-C₁-6-alkyl, 5-14-membered heteroaryl, 3-14-membered heterocycloalkyl, (3-14-membered heterocycloalkyl)-C(O)—, and —C(O)NR⁵R⁶; wherein said C₃-C₁₀-cycloalkyl, 5-14-membered heteroaryl and 3-14-membered heterocycloalkyl are optionally substituted by 1-3 substituents that are each independently selected from halogen, cyano, hydroxy, oxo, amino, C₁-C₆-alkyl, C₁-C₆-alkoxy, halo-C₁-C₆-alkyl, and halo-C₁-C₆-alkoxy;
R² is selected from hydrogen, C₁-C₆-alkyl, and C₁-C₆-alkoxy;
R³ is selected from chloro and fluoro;
R⁴ is selected from C₁-C₆-alkyl, halo-C₁-C₂-alkyl, and cyano; and
R⁵ and Re are each independently selected from hydrogen, C₁-C₆-alkyl, and hydroxy-C₁-C₆-alkyl.

2. The compound of formula (I) according to claim 1, or a pharmaceutically acceptable salt thereof, wherein

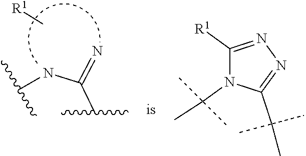

3. The compound of formula (I) according to claim 1, or a pharmaceutically acceptable salt thereof, wherein:
R¹ is selected from C₁-C₆-alkyl, hydroxy-C₁-C₆-alkyl, C₃-Cio-cycloalkyl, 5-14-membered heteroaryl, (3-14-membered heterocycloalkyl)-C(O)—, and —C(O)NR⁵R⁶; wherein said 3-14-membered heterocycloalkyl is substituted by 2 oxo substituents;
R⁵ is hydroxy -C₁-C₆-alkyl; and
R⁶ is hydrogen.

4. The compound of formula (I) according to claim 3, or a pharmaceutically acceptable salt thereof, wherein R¹ is C₁-C₆-alkyl.

5. The compound of formula (I) according to claim 4, or a pharmaceutically acceptable salt thereof, wherein R¹ is methyl.

6. The compound of formula (I) according to claim 1, or a pharmaceutically acceptable salt thereof, wherein R² is selected from hydrogen and C₁-C₆-alkyl.

7. The compound of formula (I) according to claim 6, or a pharmaceutically acceptable salt thereof, wherein R² is C₁-C₆-alkyl.

8. The compound of formula (I) according to claim 7, or a pharmaceutically acceptable salt thereof, wherein R² is methyl.

9. The compound of formula (I) according to claim 1, or a pharmaceutically acceptable salt thereof, wherein R⁴ is halo-C₁-C₆-alkyl.

10. The compound of formula (I) according to claim 9, or a pharmaceutically acceptable salt thereof, wherein R⁴ is CHF₂.

11. The compound of formula (I) according to claim 1, or a pharmaceutically acceptable salt thereof, wherein:
R¹ is selected from C₁-C₆-alkyl, hydroxy-C₁-C₆-alkyl, C₃-C₁₀-cycloalkyl, 5-14-membered heteroaryl, (3-14-membered heterocycloalkyl)-C(O)—, and —C(O)NR⁵R⁶; wherein said 3-14-membered heterocycloalkyl is substituted by 2 oxo substituents;
R² is selected from hydrogen and C₁-C₆-alkyl;
R⁵ is hydroxy-C₁-C₆-alkyl; and
R⁶ is hydrogen.

12. The compound of formula (I) according to claim 11, or a pharmaceutically acceptable salt thereof, wherein: 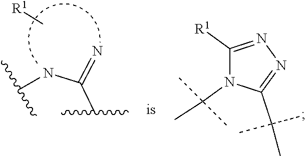
R¹ and R² are both C₁-C₆-alkyl; and
R⁴ is halo-C₁-C₆-alkyl.

13. The compound of formula (I) according to claim 12, or a pharmaceutically acceptable salt thereof, wherein:
R¹ and R² are both methyl; and
R⁴ is CHF₂.

14. The compound of formula (I) according to claim 1, or a pharmaceutically acceptable salt thereof, wherein said compound of formula (I) is selected from:
(13R)-9-(2-chloro-6-fluoro-phenyl)-3-methyl-16-thia-2,4,5,8-tetrazatetracyclo [8.6.0.02,6.011, 15]hexadeca-1(10),3,5,8, 11(15)-pentaene-13-carbonitrile;
(13R)-9-(2,6-difluorophenyl)-3-methyl-16-thia-2,4,5,8-tetrazatetracyclo [8.6.0.02,6.011, 15]hexadeca-1(10),3,5,8, 11(15)-pentaene-13-carbonitrile;
(13R)-13-(difluoromethyl)-9-(2,6-difluorophenyl)-3-methyl-16-thia-2,4,5,8-tetrazatetracyclo [8.6.0.02,6.011, 15]hexadeca-1(10),3,5,8,11(15)-pentaene;
(13R)-9-(2-chloro-6-fluoro-phenyl)-13-(difluoromethyl)-3-methyl-16-thia-2,4,5,8-tetrazatetracyclo [8.6.0.02,6.011, 15]hexadeca-1(10),3,5,8, 11(15)-pentaene;
(7S,13R)-13-(difluoromethyl)-9-(2,6-difluorophenyl)-3,7-dimethyl-16-thia-2,4,5,8-tetrazatetracyclo [8.6.0.02,6.011, 15]hexadeca-1(10),3,5,8, 11(15)-pentaene;
(13R)-9-(2-chloro-6-fluoro-phenyl)-3-cyclopropyl-13-(difluoromethyl)-16-thia-2,4,5,8-tetrazatetracyclo [8.6.0.02,6.011, 15]hexadeca-1(10),3,5,8,11(15)-pentaene;
(13R)-3-cyclopropyl-13-(difluoromethyl)-9-(2,6-difluorophenyl)-16-thia-2,4,5,8-tetrazatetracyclo [8.6.0.02,6.011, 15]hexadeca-1(10),3,5,8,11(15)-pentaene:
(7S, 13R)-3-cyclopropyl-13-(difluoromethyl)-9-(2,6-difluorophenyl)-7-methyl-16-thia-2,4,5,8-tetrazatetracyclo [8.6.0.02,6.011,15]hexadeca-1(10),3,5,8,11(15)-pentaene:
(7S, 13R)-9-(2-chloro-6-fluoro-phenyl)-13-(difluoromethyl)-3,7-dimethyl-16-thia-2,4,5,8-tetrazatetracyclo [8.6.0.02,6.011, 15]hexadeca-1(10),3,5,8,11(15)-pentaene:
(13R)-13-(difluoromethyl)-9-(2,6-difluorophenyl)-3-pyridazin-3-yl-16-thia-2,4,5,8-tetrazatetracyclo [8.6.0.02,6.011, 15]hexadeca-1(10),3,5,8, 11(15)-pentaene:
(7S, 13R)-9-(2-chloro-6-fluoro-phenyl)-3-cyclopropyl-13-(difluoromethyl)-7-methyl-16-thia-2,4,5,8-tetrazatetracyclo [8.6.0.02,6.011,15]hexadeca-1(10),3,5,8,11(15)-pentaene:
(7S, 13R)-9-(2-chloro-6-fluoro-phenyl)-13-(difluoromethyl)-7-methyl-3-pyridazin-3-yl-16-thia-2,4,5,8-tetrazatetracyclo [8.6.0.02,6.011, 15]hexadeca-1(10),3,5,8,11(15)-pentaene:
(7S, 13R)-9-(2,6-difluorophenyl)-3,7-dimethyl-13-(trifluoromethyl)-16-thia-2,4,5,8-tetrazatetracyclo [8.6.0.02,6.011, 15]hexadeca-1(10),3,5,8,11(15)-pentaene:
(4S,8R)-6-(2,6-difluorophenyl)-1,4-dimethyl-8-(trifluoromethyl)-8,9-dihydro-4H,7H-cyclopenta [4,5]thieno[3,2-f][1,2,4]triazolo[4,3-a][1,4]diazepine:
(13R)-9-(2,6-difluorophenyl)-3,13-dimethyl-16-thia-2,4,5,8-tetrazatetracyclo [8.6.0.02,6.011, 15]hexadeca-1(10),3,5,8, 11(15)-pentaene:
(7S, 13R)-9-(2,6-difluorophenyl)-3,7,13-trimethyl-16-thia-2,4,5,8-tetrazatetracyclo [8.6.0.02,6.011, 15]hexadeca-1(10),3,5,8,11(15)-pentaene:
(7S, 13R)-13-(difluoromethyl)-9-(2,6-difluorophenyl)-4,7-dimethyl-16-thia-2,3,5,8-tetrazatetracyclo [8.6.0.02,6.011, 15]hexadeca-1(10),3,5,8, 11(15)-pentaene:
(7S, 13S)-13-(difluoromethyl)-9-(2,6-difluorophenyl)-4,7-dimethyl-16-thia-2,3,5,8-tetrazatetracyclo [8.6.0.02,6.011, 15]hexadeca-1(10),3,5,8,11(15)-pentaene:
(7S,13R)-13-(difluoromethyl)-9-(2,6-difluorophenyl)-N-(2-hydroxyethyl)-7-methyl-16-thia-2,3,5,8-tetrazatetracyclo [8.6.0.02,6.011,15]hexadeca-1(10),3,5,8, 11(15)-pentaene-4-carboxamide:
[(7S,13R)-13-(difluoromethyl)-9-(2,6-difluorophenyl)-7-methyl-16-thia-2,3,5,8-tetrazatetracyclo [8.6.0.02,6.011, 15]hexadeca-1(10),3,5,8, 11(15)-pentaen-4-yl]-(1, 1-dioxo-1,4-thiazinan-4-yl)methanone:
(7S, 13R)-13-(difluoromethyl)-9-(2,6-difluorophenyl)-3,7-dimethyl-16-thia-2,5,8-triazatetracyclo [8.6.0.02,6.011,15]hexadeca-1(10),3,5,8,11(15)-pentaene:
[(7S, 13R)-13-(difluoromethyl)-9-(2,6-difluorophenyl)-7-methyl-16-thia-2,5,8-triazatetracyclo [8.6.0.02,6.011, 15]hexadeca-1(10),3,5,8, 11(15)-pentaen-3-yl]methanol: and
[(7S,13S)-13-(difluoromethyl)-9-(2,6-difluorophenyl)-7-methyl-16-thia-2,5,8-triazatetracyclo [8.6.0.02,6.011,15]hexadeca-1(10),3,5,8,11(15)-pentaen-3-yl]methanol.

15. The compound of formula (I) according to claim 14, or a pharmaceutically acceptable salt thereof, wherein said compound of formula (I) is selected from:
(7S, 13R)-13-(difluoromethyl)-9-(2,6-difluorophenyl)-3,7-dimethyl-16-thia-2,4,5,8-tetrazatetracyclo [8.6.0.02,6.011, 15]hexadeca-1(10),3,5,8, 11(15)-pentaene; and
(7S, 13R)-9-(2-chloro-6-fluoro-phenyl)-13-(difluoromethyl)-3,7-dimethyl-16-thia-2,4,5,8-tetrazatetracyclo [8.6.0.02,6.011, 15]hexadeca-1(10),3,5,8,11(15)-pentaene.

16. A pharmaceutical composition comprising a compound of formula (I) according to claim 1, or a pharmaceutically acceptable salt thereof, and a therapeutically inert carrier.

17. A method for treating or preventing acute neurological disorders, chronic neurological disorders and/or cognitive disorders in a subject, said method comprising administering an effective amount of a compound of formula (I) according to claim 1, or a pharmaceutically acceptable salt thereof.

18. The method according to claim 17, wherein said acute neurological disorders, chronic neurological disorders and/or cognitive disorders are selected from autism spectrum disorders (ASD), Angelman syndrome, age-related cognitive decline, Rett syndrome, Prader-Willi syndrome, amyotrophic lateral sclerosis (ALS), fragile-X disorder, negative and/or cognitive symptoms associated with schizophrenia, tardive dyskinesia, anxiety, social anxiety disorder (social phobia), panic disorder, agoraphobia, generalized anxiety disorder, disruptive, impulse-control and conduct disorders, Tourette's syndrome (TS), obsessive-compulsive disorder (OCD), acute stress disorder, post-traumatic stress disorder (PTSD), attention deficit hyperactivity disorder (ADHD), sleep disorders, Parkinson's disease (PD), Huntington's chorea, Alzheimer's disease (AD), mild cognitive impairment (MCI), dementia, behavioral and psychological symptoms (BPS) in neurodegenerative conditions, multi-infarct dementia, agitation, psychosis, substance-induced psychotic disorder, aggression, eating disorders, depression, chronic apathy, anhedonia, chronic fatigue, seasonal affective disorder, postpartum depression, drowsiness, sexual dysfunction, bipolar disorders, epilepsy and pain.
